SEQUENCE LISTING (85 sequences)

SEQ ID NO: 1 - DNA, 705 bp, Artificial Sequence
  Description of Artificial Sequence Synthetic polynucleotide
atgaggctccctgctcagctcctggggctcctgctgctctggttcccaggttccagatgcgacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 60 bp, Artificial Sequence
  Description of Artificial Sequence Synthetic oligonucleotide
atgaggctccctgctcagctcctggggctcctgctgctctggttcccaggttccagatgc

SEQ ID NO: 3 - DNA, 320 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaa

SEQ ID NO: 4 - protein, 234 aa, Artificial Sequence
  Description of Artificial Sequence Synthetic polypeptide
MRLPAQLLGLLLLWFPGSRCDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 20 aa, Artificial Sequence
  Description of Artificial Sequence Synthetic peptide
MRLPAQLLGLLLLWFPGSRC

SEQ ID NO: 6 - protein, 107 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 7 - DNA, 1416 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcgtggggggaccgtcagtcttcctcctgcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcctgaggagcagtacaacagcacgctgcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctctggtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 11 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgccgctaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 57 bp, Artificial Sequence
  Description of Artificial Sequence Synthetic oligonucleotide
atggactggacctggaggatcctcttcttggtggcagcagccacaggagcccactcc

SEQ ID NO: 13 - DNA, 379 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcag

SEQ ID NO: 14 - protein, 471 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 17 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 19 aa, Artificial Sequence
  Description of Artificial Sequence Synthetic peptide
MDWTWRILFLVAAATGAHS

SEQ ID NO: 20 - protein, 126 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 21 - protein, 5 aa, Unknown
  Description of Unknown Recognition motif sequence
LXPTG

SEQ ID NO: 22 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 8 aa, Artificial Sequence
  Description of Artificial Sequence Synthetic peptide
GYTFTGYY

SEQ ID NO: 24 - protein, 8 aa, Artificial Sequence
  Description of Artificial Sequence Synthetic peptide
INPDSGGT

SEQ ID NO: 25 - protein, 19 aa, Artificial Sequence
  Description of Artificial Sequence Synthetic peptide
ARDQPLGYCTNGVCSYFDY

SEQ ID NO: 26 - protein, 214 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 6 aa, Artificial Sequence
  Description of Artificial Sequence Synthetic peptide
QGIYSW

SEQ ID NO: 28 - protein, 3 aa, Artificial Sequence
  Description of Artificial Sequence Synthetic peptide
TAS

SEQ ID NO: 29 - protein, 9 aa, Artificial Sequence
  Description of Artificial Sequence Synthetic peptide
QQANIFPLT

SEQ ID NO: 30 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 8 aa, Artificial Sequence
  Description of Artificial Sequence Synthetic peptide
GFTFTDYT

SEQ ID NO: 32 - protein, 8 aa, Artificial Sequence
  Description of Artificial Sequence Synthetic peptide
VNPNSGGS

SEQ ID NO: 33 - protein, 12 aa, Artificial Sequence
  Description of Artificial Sequence Synthetic peptide
ARNLGPSFYFDY

SEQ ID NO: 34 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 6 aa, Artificial Sequence
  Description of Artificial Sequence Synthetic peptide
QDVSIG

SEQ ID NO: 36 - protein, 3 aa, Artificial Sequence
  Description of Artificial Sequence Synthetic peptide
SAS

SEQ ID NO: 37 - protein, 9 aa, Artificial Sequence
  Description of Artificial Sequence Synthetic peptide
QQYYIYPYT

SEQ ID NO: 38 - protein, 444 aa, Artificial Sequence
  Description of Artificial Sequence Synthetic polypeptide
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 8 aa, Artificial Sequence
  Description of Artificial Sequence Synthetic peptide
GYSFTGYY

SEQ ID NO: 40 - protein, 8 aa, Artificial Sequence
  Description of Artificial Sequence Synthetic peptide
VIPNAGGT

SEQ ID NO: 41 - protein, 7 aa, Artificial Sequence
  Description of Artificial Sequence Synthetic peptide
AREGIYW

SEQ ID NO: 42 - protein, 219 aa, Artificial Sequence
  Description of Artificial Sequence Synthetic polypeptide
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 11 aa, Artificial Sequence
  Description of Artificial Sequence Synthetic peptide
QSLVHSNGNTF

SEQ ID NO: 44 - protein, 3 aa, Artificial Sequence
  Description of Artificial Sequence Synthetic peptide
TVS

SEQ ID NO: 45 - protein, 9 aa, Artificial Sequence
  Description of Artificial Sequence Synthetic peptide
SQTTHVPWT

SEQ ID NO: 46 - protein, 448 aa, Artificial Sequence
  Description of Artificial Sequence Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 10 aa, Artificial Sequence
  Description of Artificial Sequence Synthetic peptide
GGSISSPGYY

SEQ ID NO: 48 - protein, 7 aa, Artificial Sequence
  Description of Artificial Sequence Synthetic peptide
IYKSGST

SEQ ID NO: 49 - protein, 13 aa, Artificial Sequence
  Description of Artificial Sequence Synthetic peptide
TRPVVRYFGWFDP

SEQ ID NO: 50 - protein, 213 aa, Artificial Sequence
  Description of Artificial Sequence Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 6 aa, Artificial Sequence
  Description of Artificial Sequence Synthetic peptide
QGISSA

SEQ ID NO: 52 - protein, 3 aa, Artificial Sequence
  Description of Artificial Sequence Synthetic peptide
DAS

SEQ ID NO: 53 - protein, 8 aa, Artificial Sequence
  Description of Artificial Sequence Synthetic peptide
QQFNSYPT

SEQ ID NO: 54 - protein, 450 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 8 aa, Artificial Sequence
  Description of Artificial Sequence Synthetic peptide
GFTFSSYG

SEQ ID NO: 56 - protein, 8 aa, Artificial Sequence
  Description of Artificial Sequence Synthetic peptide
ISYEESNR

SEQ ID NO: 57 - protein, 13 aa, Artificial Sequence
  Description of Artificial Sequence Synthetic peptide
ARDGGIAAPGPDY

SEQ ID NO: 58 - protein, 219 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 11 aa, Artificial Sequence
  Description of Artificial Sequence Synthetic peptide
QSLLYSNGYNY

SEQ ID NO: 60 - protein, 3 aa, Artificial Sequence
  Description of Artificial Sequence Synthetic peptide
LGS

SEQ ID NO: 61 - protein, 9 aa, Artificial Sequence
  Description of Artificial Sequence Synthetic peptide
MQARQTPFT

SEQ ID NO: 62 - protein, 447 aa, Artificial Sequence
  Description of Artificial Sequence Synthetic polypeptide
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCNAVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 8 aa, Artificial Sequence
  Description of Artificial Sequence Synthetic peptide
GFTFSTYG

SEQ ID NO: 64 - protein, 8 aa, Artificial Sequence
  Description of Artificial Sequence Synthetic peptide
ISGGSSYI

SEQ ID NO: 65 - protein, 12 aa, Artificial Sequence
  Description of Artificial Sequence Synthetic peptide
ARILRGGSGMDL

SEQ ID NO: 66 - protein, 217 aa, Artificial Sequence
  Description of Artificial Sequence Synthetic polypeptide
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 9 aa, Artificial Sequence
  Description of Artificial Sequence Synthetic peptide
SSNIGAGYN

SEQ ID NO: 68 - protein, 3 aa, Artificial Sequence
  Description of Artificial Sequence Synthetic peptide
GNI

SEQ ID NO: 69 - protein, 11 aa, Artificial Sequence
  Description of Artificial Sequence Synthetic peptide
AAWDKSISGLV

SEQ ID NO: 70 - protein, 120 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 71 - protein, 8 aa, Artificial Sequence
  Description of Artificial Sequence Synthetic peptide
GFSFSSTY

SEQ ID NO: 72 - protein, 8 aa, Artificial Sequence
  Description of Artificial Sequence Synthetic peptide
IYTGDGTN

SEQ ID NO: 73 - protein, 14 aa, Artificial Sequence
  Description of Artificial Sequence Synthetic peptide
ARPDITYGFAINFW

SEQ ID NO: 74 - protein, 108 aa, Artificial Sequence
  Description of Artificial Sequence Synthetic polypeptide
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 75 - protein, 6 aa, Artificial Sequence
  Description of Artificial Sequence Synthetic peptide
QSISSR

SEQ ID NO: 76 - protein, 3 aa, Artificial Sequence
  Description of Artificial Sequence Synthetic peptide
RAS

SEQ ID NO: 77 - protein, 10 aa, Artificial Sequence
  Description of Artificial Sequence Synthetic peptide
QCTGYGISWP

SEQ ID NO: 78 - protein, 117 aa, Artificial Sequence
  Description of Artificial Sequence Synthetic polypeptide
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTL

SEQ ID NO: 79 - protein, 8 aa, Artificial Sequence
  Description of Artificial Sequence Synthetic peptide
GYTFTEYI

SEQ ID NO: 80 - protein, 8 aa, Artificial Sequence
  Description of Artificial Sequence Synthetic peptide
IIPNNGGT

SEQ ID NO: 81 - protein, 15 aa, Artificial Sequence
  Description of Artificial Sequence Synthetic peptide
TRREVYGRNYYALDY

SEQ ID NO: 82 - protein, 107 aa, Artificial Sequence
  Description of Artificial Sequence Synthetic polypeptide
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 83 - protein, 6 aa, Artificial Sequence
  Description of Artificial Sequence Synthetic peptide
QGINNY

SEQ ID NO: 84 - protein, 3 aa, Artificial Sequence
  Description of Artificial Sequence Synthetic peptide
YTS

SEQ ID NO: 85 - protein, 9 aa, Artificial Sequence
  Description of Artificial Sequence Synthetic peptide
QQYSNLPYT

(19) United States
(12) Patent Application Publication
(10) Pub. No.: US 2017/0158772 A1
(43) Pub. Date: Jun. 8, 2017

(54) COMPOSITIONS OF ANTIBODY CONSTRUCT - AGONIST CONJUGATES AND METHODS OF USE THEREOF

(71) Applicant: OPI VI - IP HoldCo LLC, New York, NY (US)

(72) Inventors: Peter Armstrong Thompson, Bellevue, WA (US); Badreddin Edris, San Jose, CA (US)

(21) Appl. No.: 15/173,075

(22) Filed: Jun. 3, 2016

Related U.S. Application Data
(60) Provisional application No. 62/264,260, filed on Dec. 7, 2015.

(51) Int. Cl. C07K 16/28; A61K 47/48

(52) U.S. Cl. CPC C07K 16/2878; A61K 47/48384; C07K 2317/92

(57) ABSTRACT

Various antibody construct compositions are disclosed. The compositions of antibody construct-immune stimulatory compound conjugates are also provided. Additionally provided are the methods of preparation and used of the antibody construct-immune stimulatory compound conjugates. This includes methods for treating disorders, such as cancer.

### CROSS REFERENCE

This application claims the benefit of U.S. Provisional Patent Application No. 62/264,260, filed on Dec. 7, 2015, which is incorporated herein by reference in its entirety.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### SEQUENCE LISTING

The instant application contains a Sequence Listing which has been submitted electronically in ASCII format and is hereby incorporated by reference in its entirety. Said ASCII copy, created on Jun. 1, 2016, is named 49353-702.201_SL.txt and is 90,375 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, this invention provides a conjugate comprising: a) an immune-stimulatory compound; b) an antibody construct comprising an antigen binding domain and an Fc domain, wherein an affinity of said antigen binding domain to a first antigen in a presence of said immune-stimulatory compound is at least about 80% of an affinity of said antigen binding domain to said first antigen in an absence of said immune-stimulatory compound; and wherein an affinity of said Fc domain to an Fc receptor in a presence of said immune-stimulatory compound is at least about 80% of an affinity of said Fc domain to said Fc receptor in an absence of said immune stimulatory compound; and c) a linker attaching said antibody construct to said immune-stimulatory compound, wherein said linker is covalently bound to said antibody construct and said linker is covalently bound to said immune-stimulatory compound.

In some embodiments, this invention comprises a conjugate comprising: a) an immune-stimulatory compound; and b) an antibody construct comprising an antigen binding domain and an Fc domain, wherein a specificity of the antigen binding domain to a first antigen in the presence of said immune stimulatory compound is at least about 80% of a specificity of the antigen binding domain to the first antigen in the absence of said immune stimulatory compound; and wherein a specificity of the Fc domain to an Fc receptor in the presence of said immune stimulatory compound is at least about 80% of a specificity of the Fc domain to the Fc receptor in the absence of said immune stimulatory compound.

In some embodiments, this invention comprises a conjugate comprising: a) an immune-stimulatory compound; and b) an antibody construct comprising an antigen binding domain and an Fc domain, wherein an affinity of the antigen binding domain to a first antigen in the presence of said immune stimulatory compound is at least about 80% of an affinity of the antigen binding domain to the first antigen in the absence of said immune stimulatory compound; and wherein an affinity of the Fc domain to an Fc receptor in the presence of said immune stimulatory compound is at least about 80% of an affinity of the Fc domain to the Fc receptor in the absence of said immune stimulatory compound.

In some embodiments, this invention comprises a conjugate comprising: a) an immune-stimulatory compound; and b) an antibody construct comprising an antigen binding domain and an Fc domain, wherein a specificity of the antigen binding domain to a first antigen in the presence of said immune stimulatory compound is at least about 80% of a specificity of the antigen binding domain to the first antigen in the absence of said immune stimulatory compound; and wherein an affinity of the Fc domain to an Fc receptor in the presence of said immune stimulatory compound is at least about 80% of an affinity of the Fc domain to the Fc receptor in the absence of said immune stimulatory compound.

In some embodiments, this invention comprises a conjugate comprising: a) an immune-stimulatory compound; and b) an antibody construct comprising an antigen binding domain and an Fc domain, wherein an affinity of the antigen binding domain to a first antigen in the presence of said immune stimulatory compound is at least about 80% of an affinity of the antigen binding domain to the first antigen in the absence of said immune stimulatory compound; and wherein a specificity of the Fc domain to an Fc receptor in the presence of said immune stimulatory compound is at least about 80% of a specificity of the Fc domain to the Fc receptor in the absence of said immune stimulatory compound.

In some embodiments, this invention comprises an immune cell bound to a conjugate comprising: a):an immune-stimulatory compound; and b) an antibody construct comprising an antigen binding domain and an Fc domain, wherein a specificity of the antigen binding domain to a first antigen in the presence of said immune stimulatory compound is at least about 80% of a specificity of the antigen binding domain to the first antigen in the absence of said immune stimulatory compound; and wherein a specificity of the Fc domain to an Fc receptor in the presence of said immune stimulatory compound is at least about 80% of a specificity of the Fc domain to the Fc receptor in the absence of said immune stimulatory compound.

In some embodiments, this invention comprises: a) an immune-stimulatory compound; b) an antibody construct comprising an antigen binding domain and an Fc domain, wherein: i) a K_{d} for binding of said antigen binding domain to a first antigen in a presence of said immune-stimulatory compound is less than about 100 nM and no greater than than about 100 times a K_{d} for binding of said antigen binding domain to said first antigen in the absence of said immune-stimulatory compound, ii) a K_{d} for binding of said Fc domain to an Fc receptor in the presence of said immune-stimulatory compound is no greater than about 100 times a K_{d} for binding of said Fc domain to said Fc receptor in the absence of the immune stimulatory compound, and iii) a molar ratio of immune-stimulatory compound to antibody construct is less than 8; and c) a linker attaching said antibody construct to said immune-stimulatory compound, wherein said linker is covalently bound to said antibody construct and said linker is covalently bound to said immune-stimulatory compound. In some aspects, said K_{d} for binding of said antigen binding domain to said first antigen in the presence of said immune-stimulatory compound is less than about 100 nM and is no greater than about 10 times the K_{d} of the binding of the antigen binding domain to said first antigen in the absence of the immune-stimulatory compound; and said K_{d} for binding of said Fc domain to said Fc receptor in the presence of said immune-stimulatory compound is no greater than about 10 times said K_{d} for the binding of said Fc domain to said Fc receptor in the absence of said immune stimulatory compound. In other aspects, said molar ratio of immune-stimulatory compound to antibody is less than 5.

In some embodiments, this invention comprises a conjugate comprising: a) an immune-stimulatory compound, wherein said immune-stimulatory compound is conjugated to an antibody construct via a linker; and b) said antibody construct comprises an antigen binding domain and an Fc domain, wherein said antigen binding domain specifically binds a first antigen in the presence of said immune stimulatory compound; and wherein said Fc domain specifically binds an Fc receptor in the presence of said immune-stimulatory compound.

In some embodiments, this invention comprises a conjugate, comprising: an antibody construct comprising an antigen binding domain and an Fc domain; an immune stimulatory compound; and a linker attaching said antibody construct to said immune-stimulatory compound, wherein said linker is covalently bound to said antibody construct and said linker is covalently bound to said immune-stimulatory compound; wherein said antigen binding domain specifically binds a first antigen and said Fc domain specifically binds an Fc receptor.

The conjugate of any of the preceding embodiments, wherein said antibody construct further comprises a targeting binding domain. In some aspects, the targeting binding domain specifically binds a second antigen. In some aspects, said targeting binding domain is conjugated to said antibody construct at a C-terminal end of said Fc domain.

In some embodiments, this invention comprises a conjugate comprising: a) an immune-stimulatory compound; and b) an antibody construct comprising an antigen binding domain and a targeting domain, wherein said antigen binding domain specifically binds a first antigen in the presence of said immune-stimulatory compound; and wherein said targeting domain specifically binds a second antigen in the presence of said immune-stimulatory compound.

In some embodiments, this invention comprises a conjugate comprising: a) an immune-stimulatory compound, wherein said immune-stimulatory compound is conjugated to an antibody construct via a linker; and b) said antibody construct comprises an antigen binding domain and a targeting domain, wherein said antigen binding domain specifically binds a first antigen in the presence of said immune stimulatory compound; and wherein said targeting domain specifically binds a second antigen in the presence of said immune-stimulatory compound.

In some embodiments, this invention comprises a conjugate, comprising: an antibody construct comprising an antigen binding domain and a targeting domain; an immune stimulatory compound; and a linker attaching said antibody construct to said immune-stimulatory compound, wherein said linker is covalently bound to said antibody construct and said linker is covalently bound to said immune-stimulatory compound; wherein said antigen binding domain specifically binds a first antigen and said targeting domain specifically binds a second antigen.

The conjugate of any one of the preceding embodiments, wherein said antibody construct comprises an Fc domain. In some aspects, said Fc domain specifically binds an Fc receptor in the presence of said immune-stimulatory compound. In some aspects, said targeting binding domain is conjugated to said antibody construct at a C-terminal end of said Fc domain.

The conjugate of any one of the preceding embodiments, wherein said antigen binding domain is from an antibody or non-antibody scaffold.

The conjugate of any one of the preceding embodiments, wherein said antigen binding domain is at least 80% homologous to an antigen binding domain from an antibody or non-antibody scaffold.

The conjugate of any one of the preceding embodiments, wherein said antigen binding domain is from DARPins, affimers, avimers, knottins, monobodies, or affinity clamps.

The conjugate of any one of the preceding embodiments, wherein said antigen binding domain is at least 80% homologous to an antigen binding domain from DARPins, affimers, avimers, knottins, monobodies, or affinity clamps.

The conjugate of any one of the preceding embodiments, wherein said antigen binding domain recognizes a single antigen.

The conjugate of any one of the preceding embodiments, wherein said antigen binding domain recognizes two or more antigens.

The conjugate of any one of the preceding embodiments, wherein said first antigen is a tumor antigen.

The conjugate of any one of the preceding embodiments, wherein said first antigen is CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1.

The conjugate of any one of the preceding embodiments, wherein said first antigen is expressed on an immune cell.

The conjugate of any one of the preceding embodiments, wherein said first antigen is expressed on an antigen-presenting cell.

The conjugate of any one of the preceding embodiments, wherein said first antigen is expressed on a dendritic cell, a macrophage, or a B-cell.

The conjugate of any one of the preceding embodiments, wherein said first antigen is CD40.

The conjugate of any one of the preceding embodiments, wherein said antigen binding domain is a CD40 agonist.

The conjugate of any one of the preceding embodiments, wherein said second antigen is expressed on an immune cell.

The conjugate of any one of the preceding embodiments, wherein said second antigen is expressed on an antigen-presenting cell.

The conjugate of any one of the preceding embodiments, wherein said second antigen is expressed on a dendritic cell, a macrophage, or a B-cell.

The conjugate of any one of the preceding embodiments, wherein said second antigen is CD40.

The conjugate of any one of the preceding embodiments, wherein said targeting binding domain is CD40.

The conjugate of any one of the preceding embodiments, wherein said targeting binding domain is a CD40 agonist.

The conjugate of any one of the preceding embodiments, wherein said antibody construct is an antibody.

The conjugate of any one of the preceding embodiments, wherein said antibody construct is a human antibody or a humanized antibody.

The conjugate of any one of the preceding embodiments, wherein said antibody construct comprises a light chain sequence that is at least 90% homologous to SEQ ID NO: 4.

The conjugate of any one of the preceding embodiments, wherein said antibody construct comprises a variable domain sequence that is at least 90% homologous to SEQ ID NO: 6.

The conjugate of any one of the preceding embodiments, wherein said antibody construct comprises a heavy chain sequence that is at least 90% homologous to SEQ ID NO: 15.

The conjugate of any one of the preceding embodiments, wherein said antibody construct comprises a variable domain that is at least 90% homologous to SEQ ID NO: 20.

The conjugate of any one of the preceding embodiments, wherein said antibody construct comprises a heavy chain sequence that is at least 90% homologous to SEQ ID NO: 16.

The conjugate of any one of the preceding embodiments, wherein said antibody construct comprises a variable domain that is at least 90% homologous to SEQ ID NO: 20.

The conjugate of any one of the preceding embodiments, wherein said antibody construct comprises a heavy chain sequence that is at least 90% homologous to SEQ ID NO: 17.

The conjugate of any one of the preceding embodiments, wherein said antibody construct comprises a variable domain that is at least 90% homologous to SEQ ID NO: 20.

The conjugate of any one of the preceding embodiments, wherein said antibody construct comprises a heavy chain sequence that is at least 90% homologous to SEQ ID NO: 18.

The conjugate of any one of the preceding embodiments, wherein a complementarity determining region of the antigen binding domain is selected from a group consisting of a light chain sequence that is at least 80% homologous to the SEQ ID NO: 27, a light chain sequence that is at least 80% homologous to SEQ ID NO: 28, a light chain sequence that is at least 80% homologous to SEQ ID NO: 29, a heavy chain sequence that is at least 80% homologous to SEQ ID NO: 23, a heavy chain sequence that is at least 80% homologous to SEQ ID NO: 24, and a heavy chain sequence that is at least 80% homologous to SEQ ID NO: 25.

The conjugate of any one of the preceding embodiments, wherein a complementarity determining region of the antigen binding domain is selected from a group consisting of a light chain sequence that is at least 80% homologous to the SEQ ID NO: 35, a light chain sequence that is at least 80% homologous to SEQ ID NO: 36, a light chain sequence that is at least 80% homologous to SEQ ID NO: 37, a heavy chain sequence that is at least 80% homologous to SEQ ID NO: 31, a heavy chain sequence that is at least 80% homologous to SEQ ID NO: 32, and a heavy chain sequence that is at least 80% homologous to SEQ ID NO: 33.

The conjugate of any one of the preceding embodiments, wherein said Fc domain is from an antibody.

The conjugate of any one of the preceding embodiments, wherein said Fc domain is at least 80% homologous to an Fc domain from an antibody.

The conjugate of any one of the preceding embodiments, wherein said Fc domain binding to said Fc receptor in the presence of said immune-stimulatory compound results in Fc-receptor-mediated signaling.

The conjugate of any one of the preceding embodiments, wherein said Fc domain is a human Fc domain.

The conjugate of any one of the preceding embodiments, wherein said Fc domain is selected from a group consisting of a human IgG1 Fc domain, a human IgG2 Fc domain, a human IgG3 Fc domain, a human IgG4 Fc domain, a human IgA1 Fc domain, a human IgA2 Fc domain, a human, IgE Fc domain, a human IgD Fc domain, a human IgM Fc domain.

The conjugate of any one of the preceding embodiments, wherein said Fc domain is an Fc domain variant comprising at least one amino acid residue change as compared to a wild type sequence of said Fc domain.

The conjugate of any one of the preceding embodiments, wherein said Fc domain binds said Fc receptor with altered affinity as compared to a wild type Fc domain.

The conjugate of any one of the preceding embodiments, wherein said Fc receptor is selected from a group consisting of FcRn, CD89, FcαμR, CD16a, CD16b, CD32a, CD32b, and CD64.

The conjugate of any one of the preceding embodiments, wherein said Fc receptor is a CD16a F158 variant or a CD16a V158 variant.

The conjugate of any one of the preceding embodiments, wherein said Fc domain binds said Fc receptor with higher affinity than a wild type Fc domain.

The conjugate of any one of the preceding embodiments, wherein said Fc receptor is selected from a group consisting of: FcRn, CD16a, CD16b, CD32a, CD32b, CD89, FcαμR, or CD64.

The conjugate of any one of the preceding embodiments, wherein said Fc receptor is a CD16a F158 variant or a CD16a V158 variant. In some aspects, said at least one amino acid residue is F243L, R292P, Y300L, L235V, and P396L, wherein numbering of amino acid residues in said Fc domain is according to the EU index as in Kabat. In some aspects, said at least one amino acid residue is S239D and 1332E, wherein numbering of amino acid residues in said Fc domain is according to the EU index as in Kabat. In some aspects, said at least one amino acid residue is S298A, E333A, and K334A, wherein numbering of amino acid residues in said Fc domain is according to the EU index as in Kabat.

The conjugate of any one of the preceding embodiments, wherein said immune-stimulatory compound is a damage-associated molecular pattern molecule.

The conjugate of any one of the preceding embodiments, wherein said immune-stimulatory compound is a pathogen-associated molecular pattern molecule.

The conjugate of any one of the preceding embodiments, wherein said immune-stimulatory compound is a toll-like receptor agonist.

The conjugate of any one of the preceding embodiments, wherein said immune-stimulatory compound is a STING agonist.

The conjugate of any one of the preceding embodiments, wherein said immune-stimulatory compound is a cyclic dinucleotide.

The conjugate of any one of the preceding embodiments, wherein said immune-stimulatory compound is ADU-S100.

The conjugate of any one of the preceding embodiments, wherein said immune-stimulatory agonist is a RIG-I agonist, wherein the RIG-I agonist is KIN700, KIN1148, KIN600, KIN500, KIN100, KIN101, KIN400, KIN2000, or SB-9200.

The conjugate of any one of the preceding embodiments, wherein said immune-stimulatory compound is a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist.

The conjugate of any one of the preceding embodiments, wherein said immune-stimulatory compound is selected from a group consisting of: S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, or CL663.

The conjugate of any one of the preceding embodiments, wherein said immune-stimulatory compound comprises one or more rings selected from carbocyclic and heterocyclic rings.

The conjugate of any one of the preceding embodiments, wherein said linker is not conjugated at any amino acid residue selected from a group consisting of: 221, 222, 224, 227, 230, 231, 232, 234, 235, 236, 237, 239, 240, 243, 244, 245, 247, 249, 258, 262, 263, 264, 265, 266, 267, 268, 269, 270, 271, 272, 273, 275, 278, 280, 281, 283, 285, 286, 288, 290, 291, 292, 293, 294, 295, 296, 297, 298, 299, 300, 305, 313, 317, 320, 322, 323, 324, 325, 326, 327, 328, 329, 330, 331, 332, 333, 334, 335, 336, 396, or 428, wherein numbering of amino acid residues in said Fc domain is according to the EU index as in Kabat, wherein the linker is not conjugated to the amino acid residues bracketing the CDR domain as defined by Kabat numbering.

The conjugate of any one of the preceding embodiments, wherein said linker is conjugated to an amino acid residue of said antibody construct by a THIOMAB linker, or a Sortase A-catalyzed linker.

The conjugate of any one of the preceding embodiments, wherein said linker is conjugated to said antibody construct via a sulfhydryl group.

The conjugate of any one of the preceding embodiments, wherein said linker is conjugated to said antibody construct via a primary amine.

The conjugate of any one of the preceding embodiments, wherein said linker is conjugated to said antibody construct via a hinge cysteine, a C_{L} lysine, an engineered cysteine in a light chain, an engineered light chain glutamine, or an unnatural amino acid engineered into a light chain or heavy chain.

The conjugate of any one of the preceding embodiments, wherein said linker is conjugated to said antibody construct at an amino acid residue, wherein a conjugation at said amino acid residue does not interfere with said Fc domain binding to said Fc receptor.

The conjugate of any one of the preceding embodiments, wherein said linker is conjugated to said antibody at an amino acid residue, wherein a conjugation at said amino acid residue does not interfere with Fc-receptor-mediated signaling resulting from said Fc domain binding to said Fc receptor.

The conjugate of any one of the preceding embodiments, wherein said linker is conjugated to said immune-stimulatory compound via an exocyclic nitrogen or carbon atom of said immune-stimulatory compound.

The conjugate of any one of the preceding embodiments, wherein said immune-stimulatory compound is covalently bound to said linker by a bond to an exocyclic carbon or nitrogen atom on said immune-stimulatory compound.

The conjugate of any one of the preceding embodiments, wherein said linker is a cleavable linker.

The conjugate of any one of the preceding embodiments, wherein said linker is a valine-citrulline linker.

The conjugate of any one of the preceding embodiments, wherein said linker is a valine-citrulline linker containing a pentafluorophenyl group.

The conjugate of any one of the preceding embodiments, wherein said linker is a valine-citrulline linker containing a succinimide group.

The conjugate of any one of the preceding embodiments, wherein said linker is a valine-citrulline linker containing a para aminobenzoic acid group.

The conjugate of any one of the preceding embodiments, wherein said linker is a valine-citrulline linker containing a para aminobenzoic acid group and a pentafluorophenyl group.

The conjugate of any one of the preceding embodiments, wherein said linker is a valine-citrulline linker containing a para aminobenzoic acid group and a succinimide group.

The conjugate of any one of the preceding embodiments, wherein said linker is a valine-alanine linker.

The conjugate of any one of the preceding embodiments, wherein said linker is a valine-alanine linker containing a pentafluorophenyl group.

The conjugate of any one of the preceding embodiments, wherein said linker is a valine-alanine linker containing a succinimide group.

The conjugate of any one of the preceding embodiments, wherein said linker is a valine-alanine linker containing a para aminobenzoic acid group.

The conjugate of any one of the preceding embodiments, wherein said linker is a valine-alanine linker containing a para aminobenzoic acid group and a pentafluorophenyl group.

The conjugate of any one of the preceding embodiments, wherein said linker is a valine-alanine linker containing a para aminobenzoic acid group and a succinimide group.

The conjugate of any one of the preceding embodiments, wherein said linker is a non-cleavable linker.

The conjugate of any one of the preceding embodiments, wherein said linker is a maleimidocaproyl linker.

The conjugate of any one of the preceding embodiments, wherein said linker is a combination of a maleimidocaproyl group and one or more polyethylene glycol molecules.

The conjugate of any one of the preceding embodiments, wherein said linker is a maleimide-PEG4 linker.

The conjugate of any one of the preceding embodiments, wherein said linker is a maleimidocaproyl linker containing a succinimide group.

The conjugate of any one of the preceding embodiments, wherein said linker is a maleimidocaproyl linker containing a pentafluorophenyl group.

The conjugate of any one of the preceding embodiments, wherein said linker is a combination of a maleimidocaproyl linker containing a succinimide group and one or more polyethylene glycol molecules.

The conjugate of any one of the preceding embodiments, wherein said linker is a combination of a maleimidocaproyl linker containing a pentafluorophenyl group and one or more polyethylene glycol molecules.

The conjugate of any one of the preceding embodiments, wherein said conjugate is formulated to treat tumors.

The conjugate of any one of the preceding embodiments, wherein said conjugate is in a pharmaceutical formulation.

A method of producing the conjugate of any one of the preceding embodiments, comprising: a) selecting an antibody construct; b) selecting an immune-stimulatory compound; and c) conjugating said antibody construct to said immune-stimulatory compound, wherein said antibody construct comprises an antigen binding domain and an Fc domain, and wherein said antigen binding domain specifically binds an antigen in the presence of said immune-stimulatory compound and said Fc domain specifically binds an Fc receptor in the presence of said immune-stimulatory compound.

A method of producing the conjugate of any one of the preceding embodiments, comprising: a) selecting an antibody construct; b) selecting an immune-stimulatory compound; and c) conjugating said antibody construct to said immune-stimulatory compound, wherein said immune-stimulatory compound is conjugated to said antibody construct via a linker and said antibody construct comprises an antigen binding domain and an Fc domain, wherein said antigen binding domain specifically binds an antigen in the presence of said immune-stimulatory compound and said Fc domain specifically binds an Fc receptor in the presence of said immune-stimulatory compound.

A method of producing the conjugate of any one of the preceding embodiments, comprising: a) selecting an antibody construct; b) selecting an immune-stimulatory compound; and c) conjugating said antibody construct to said immune-stimulatory compound, wherein said antibody construct comprises an antigen binding domain and a targeting domain, and wherein said antigen binding domain specifically binds a first antigen in the presence of said immune-stimulatory compound and said targeting domain specifically binds a second antigen in the presence of said immune-stimulatory compound.

A method of producing the conjugate of any one of the preceding embodiments, comprising: a) selecting an antibody construct; b) selecting an immune-stimulatory compound; and c) conjugating said antibody construct to said immune-stimulatory compound, wherein said immune-stimulatory compound is conjugated to said antibody construct via a linker and said antibody construct comprises an antigen binding domain and a targeting binding domain, wherein said antigen binding domain specifically binds a first antigen in the presence of said immune-stimulatory compound and said targeting binding specifically binds a second antigen in the presence of said immune-stimulatory compound.

A method for treating a tumor, comprising administering a therapeutic dose of said conjugate of any one of the preceding embodiments with a pharmaceutically acceptable carrier.

A kit comprising of said conjugate of any one of the preceding embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1A illustrates a DNA sequence (SEQ ID NO: 1) of a light chain of a human CD40 monoclonal antibody SBT-040. Furthermore, SEQ ID NO: 1 illustrates a DNA sequence containing a signal sequence (SEQ ID NO: 2) as shown in FIG. 1B and a variable domain sequence (SEQ ID NO: 3) as shown in FIG. 1C.

FIG. 1B illustrates a DNA sequence of a signal sequence (SEQ ID NO: 2) of a light chain of a human CD40 monoclonal antibody SBT-040.

FIG. 1C illustrates a DNA sequence of a variable domain (SEQ ID NO: 3) in a light chain of a human CD40 monoclonal antibody SBT-040.

FIG. 2A illustrates an amino acid sequence (SEQ ID NO: 4) of a light chain of a human CD40 monoclonal antibody SBT-040. Furthermore, SEQ ID NO: 4 illustrates an amino acid sequence containing a signal sequence (SEQ ID NO: 5) as shown in FIG. 2B and a variable domain sequence (SEQ ID NO: 6) as shown in FIG. 2C.

FIG. 2B illustrates an amino acid sequence of a signal sequence (SEQ ID NO: 5) of a light chain of a human CD40 monoclonal antibody SBT-040.

FIG. 2C illustrates an amino acid sequence of a variable domain (SEQ ID NO: 6) in a light chain of a human CD40 monoclonal antibody SBT-040.

FIG. 3A illustrates a DNA sequence (SEQ ID NO: 7) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 7 illustrates a DNA sequence containing a signal sequence (SEQ ID NO: 12) as shown in FIG. 3F and a variable domain sequence (SEQ ID NO: 13) as shown in FIG. 3G.

FIG. 3B illustrates a DNA sequence (SEQ ID NO: 8) of a wildtype IgG1 isotype heavy chain of a human CD40 monoclonal antibody SBT-040, wherein this heavy chain of the SBT-040 antibody can also be referred to as SBT-040-G1WT. Furthermore, SEQ ID NO: 8 illustrates a DNA sequence containing a signal sequence (SEQ ID NO: 12) as shown in FIG. 3F and a variable domain sequence (SEQ ID NO: 13) as shown in FIG. 3G.

FIG. 3C illustrates a DNA sequence (SEQ ID NO: 9) of an IgG1 isotype heavy chain of a human CD40 monoclonal antibody SBT-040 containing DNA nucleotide modifications corresponding to L235V, F243L, R292P, Y300L, and P396L amino acid residue modifications of a wildtype IgG1 Fc domain, wherein this heavy chain of the SBT-040 antibody can also be referred to as SBT-040-G1VLPLL. The modified DNA nucleotides corresponding to the L235V, F243L, R292P, Y300L, and P396L amino acid residue modifications are in bold. Furthermore, SEQ ID NO: 9 illustrates a DNA sequence containing a signal sequence (SEQ ID NO: 12) as shown in FIG. 3F and a variable domain sequence (SEQ ID NO: 13) as shown in FIG. 3G.

FIG. 3D illustrates a DNA sequence (SEQ ID NO: 10) of an IgG1 isotype heavy chain of a human CD40 monoclonal antibody SBT-040 containing DNA nucleotide modifications corresponding to S239D and I332E amino acid residue modifications of a wildtype IgG1 Fc domain, wherein this heavy chain of the SBT-040 antibody can also be referred to as SBT-040-G1DE. The modified DNA nucleotides corresponding to the S239D and I332E amino acid residue modifications are in bold. Furthermore, SEQ ID NO: 10 illustrates a DNA sequence containing a signal sequence (SEQ ID NO: 12) as shown in FIG. 3F and a variable domain sequence (SEQ ID NO: 13) as shown in FIG. 3G.

FIG. 3E illustrates a DNA sequence (SEQ ID NO: 11) of an IgG1 isotype heavy chain of human CD40 monoclonal antibody SBT-040 containing DNA nucleotide modifications corresponding to S298A, E333A, and K334A amino acid residue modifications of a wildtype IgG1 Fc domain, wherein this heavy chain of the SBT-040 antibody can also be referred to as SBT-040-G1AAA. The modified DNA nucleotides corresponding to the S298A, E333A, and K334A amino acid residue modifications are in bold. Furthermore, SEQ ID NO: 11 illustrates a DNA sequence containing a signal sequence (SEQ ID NO: 12) as shown in FIG. 3F and a variable domain sequence (SEQ ID NO: 13) as shown in FIG. 3G.

FIG. 3F illustrates a DNA sequence of a signal sequence (SEQ ID NO: 12) of a heavy chain of a human CD40 monoclonal antibody SBT-040.

FIG. 3G illustrates a DNA sequence of a variable domain (SEQ ID NO: 13) in a heavy chain of a human CD40 monoclonal antibody SBT-040.

FIG. 4A illustrates an amino acid sequence (SEQ ID NO: 14) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 14 illustrates an amino acid sequence containing a signal sequence (SEQ ID NO: 19) as shown in FIG. 4F and a variable domain sequence (SEQ ID NO: 20) as shown in FIG. 4G.

FIG. 4B illustrates an amino acid sequence (SEQ ID NO: 15) of a wildtype IgG1 isotype heavy chain of a human CD40 monoclonal antibody SBT-040, wherein this heavy chain of the SBT-040 antibody can also be referred to as SBT-040-G1WT. Furthermore, SEQ ID NO: 15 illustrates an amino acid sequence containing a signal sequence (SEQ ID NO: 19) as shown in FIG. 4F and a variable domain sequence (SEQ ID NO: 20) as shown in FIG. 4G.

FIG. 4C illustrates an amino acid sequence (SEQ ID NO: 16) of an IgG1 isotype heavy chain of a human CD40 monoclonal antibody SBT-040 containing L235V, F243L, R292P, Y300L, and P396L amino acid residue modifications of a wildtype IgG1 Fc domain, wherein this heavy chain of the SBT-040 antibody can also be referred to as SBT-040-G1VLPLL. The amino acid residues corresponding to the L235V, F243L, R292P, Y300L, and P396L amino acid residue modifications are in bold. Furthermore, SEQ ID NO: 16 illustrates an amino acid sequence containing a signal sequence (SEQ ID NO: 19) as shown in FIG. 4F and a variable domain sequence (SEQ ID NO: 150) as shown in FIG. 4G.

FIG. 4D illustrates an amino acid sequence (SEQ ID NO: 17) of an IgG1 isotype heavy chain of a human CD40 monoclonal antibody SBT-040 containing S239D and 1332 amino acid residue modifications of a wildtype IgG1 Fc domain, wherein this heavy chain of the SBT-040 antibody can also be referred to as SBT-040-G1DE. The amino acid residues corresponding to the S239D and I332E amino acid residue modifications are in bold. Furthermore, SEQ ID NO: 17 illustrates an amino acid sequence containing a signal sequence (SEQ ID NO: 19) as shown in FIG. 4F and a variable domain sequence (SEQ ID NO: 20) as shown in FIG. 4G.

FIG. 4E illustrates an amino acid sequence (SEQ ID NO: 18) of an IgG1 isotype heavy chain of a human CD40 monoclonal antibody SBT-040 containing S298A, E333A, and K334A amino acid residue modifications of a wildtype IgG1 Fc domain, wherein this heavy chain of the SBT-040 antibody can also be referred to as SBT-040-G1AAA. The amino acid residues corresponding to the S298A, E333A, and K334A amino acid modifications are in bold. Furthermore, SEQ ID NO: 11 illustrates an amino acid sequence containing a signal sequence (SEQ ID NO: 12) as shown in FIG. 4F and a variable domain sequence (SEQ ID NO: 13) as shown in FIG. 4G.

FIG. 4F illustrates an amino acid sequence of a signal sequence (SEQ ID NO: 12) of a heavy chain of a human CD40 monoclonal antibody SBT-040.

FIG. 4G illustrates an amino acid sequence of a variable domain (SEQ ID NO: 13) in a heavy chain of a human CD40 monoclonal antibody SBT-040.

FIGS. 5A-5C illustrate a CLUSTAL O(1.2.1) multiple DNA sequence alignment of the DNA sequences of SBT-040-G1VLPLL (SEQ ID NO: 9), SBT-040-G1AAA (SEQ ID NO: 11), SBT-040-G1WT (SEQ ID NO: 8), and SBT-040-G1DE (SEQ ID NO: 10). The SBT-040-G1VLPLL sequence is a DNA sequence of an IgG1 isotype heavy chain of a human CD40 monoclonal antibody SBT-040 containing DNA nucleotide modifications corresponding to L235V, F243L, R292P, Y300L, and P396L amino acid residue modifications of a wildtype IgG1 Fc domain. The modified DNA nucleotides corresponding to the L235V, F243L, R292P, Y300L, and P396L amino acid residue modifications are in bold. The SBT-040-G1AAA sequence is a DNA sequence of an IgG1 isotype heavy chain of a human CD40 monoclonal antibody SBT-040 containing DNA nucleotide modifications corresponding to S298A, E333A, and K334A amino acid residue modifications of a wildtype IgG1 Fc domain. The modified DNA nucleotides corresponding to the S298A, E333A, and K334A amino acid residue modifications are highlighted. The SBT-040-G1WT sequence is a DNA sequence of an IgG1 isotype heavy chain of a human CD40 monoclonal antibody SBT-040. The SBT-040-G1AAA sequence is a DNA sequence of an IgG1 isotype heavy chain of a human CD40 monoclonal antibody SBT-040 containing DNA nucleotide modifications corresponding to S239D and I332E amino acid residue modifications of a wildtype IgG1 Fc domain. The modified DNA nucleotides corresponding to the S239D and I332E amino acid residue modifications are in bold italics.

FIG. 6 illustrates a CLUSTAL O(1.2.1) multiple amino acid sequence alignment of the amino acid sequences of SBT-040-G1VLPLL (SEQ ID NO: 16), SBT-040-G1AAA (SEQ ID NO: 18), SBT-040-G1WT (SEQ ID NO: 15), and SBT-040-G1DE (SEQ ID NO: 17). The SBT-040-G1VLPLL sequence is an amino acid sequence of an IgG1 isotype heavy chain of a human CD40 monoclonal antibody SBT-040 containing L235V, F243L, R292P, Y300L, and P396L amino acid residue modifications of a wildtype IgG1 Fc domain. The L235V, F243L, R292P, Y300L, and P396L amino acid residue modifications are highlighted in yellow. The SBT-040-G1AAA sequence is an amino acid sequence of an IgG1 isotype heavy chain of a human CD40 monoclonal antibody SBT-040 containing S298A, E333A, and K334A amino acid residue modifications of a wildtype IgG1 Fc domain. The S298A, E333A, and K334A amino acid residue modifications are highlighted in blue. The SBT-040-G1WT sequence is an amino acid sequence of an IgG1 isotype heavy chain of a human CD40 monoclonal antibody SBT-040. The SBT-040-G1AAA sequence is an amino acid sequence of an IgG1 isotype heavy chain of a human CD40 monoclonal antibody SBT-040 containing S239D and I332E amino acid residue modifications of a wildtype IgG1 Fc domain. The S239D and I332E amino acid residue modifications are highlighted in green. Additionally, the hinge region of each amino acid sequence is differentiated from other regions of the amino acid sequence by colored font. The red font indicates the upper portion of the hinge region. The blue font indicates the middle portion of the hinge region. The green font indicates the lower portion of the hinge region.

FIG. 7 illustrates a schematic of an antibody. An antibody contains two heavy chains as shown in gray and two light chains as shown in light gray. A portion of the heavy chains contain Fc domains (705 and 720). An antibody contains two antigen binding sites (710 and 715).

FIG. 8 illustrates a schematic of an exemplary conjugate. An antibody construct is an antibody, which contains two heavy chains as shown in gray and two light chains as shown in light gray. The antibody comprises two antigen binding sites (810 and 815), and a portion of the heavy chains contain Fc domains (805 and 620). The immune-stimulatory compounds (830 and 840) are conjugated to the antibody by linkers (860 and 870).

FIG. 9 illustrates a schematic of an exemplary conjugate. An antibody construct is an antibody, which contains two heavy chains as shown in gray and two light chains as shown in light gray. The antibody comprises two antigen binding sites (910 and 915), and a portion of the heavy chains contain Fc domains (905 and 920). The immune-stimulatory compounds (930 and 940) are conjugated to the antibody by linkers (960 and 970). Targeting binding domains are conjugated to the antibody (980 and 985).

FIG. 10 illustrates a schematic of an exemplary conjugate. An antibody construct contains the Fc region of an antibody with the heavy chains shown in gray, and two scaffolds as shown in light gray. The antibody construct comprises two antigen binding sites (1010 and 1015) in the scaffolds, and a portion of the heavy chains contain Fc domains (1005 and 1020). The immune-stimulatory compounds (630 and 640) are conjugated to the antibody construct by linkers (1060 and 1070).

FIG. 11 illustrates a schematic of an exemplary conjugate. An antibody construct contains the Fc region of an antibody with the heavy chains shown in gray, and two scaffolds as shown in light gray. The antibody construct comprises two antigen binding sites (1110 and 1115) in the scaffolds, and a portion of the heavy chains contain Fc domains (1105 and 1120). The immune-stimulatory compounds (1130 and 1140) are conjugated to the antibody construct by linkers (1160 and 1170). Targeting binding domains are conjugated to the antibody construct (1180 and 1185).

FIG. 12 illustrates a schematic of an exemplary conjugate. An antibody construct contains the F(ab′)2 region of an antibody with heavy chains shown in gray and light chains shown in light gray, and two scaffolds as shown in dark gray. The antibody construct comprises two antigen binding sites (1210 and 1215), and a portion of two scaffolds contain Fc domains (1240 and 1245). The immune-stimulatory compounds (1230 and 1240) are conjugated to the antibody construct by linkers (1260 and 1270).

FIG. 13 illustrates a schematic of an exemplary conjugate. An antibody construct contains the F(ab′)2 region of an antibody with heavy chains shown in gray and light chains shown in light gray, and two scaffolds as shown in dark gray. The antibody construct comprises two antigen binding sites (1310 and 1315), and a portion of two scaffolds contain Fc domains (1340 and 1345). The immune-stimulatory compounds (1330 and 1340) are conjugated to the antibody construct by linkers (1360 and 1370). Targeting binding domains are conjugated to the antibody construct (1380 and 1385).

FIG. 14 illustrates a schematic of an exemplary conjugate. An antibody construct contains two scaffolds as shown in light gray and two scaffolds as shown in dark gray. The antibody construct comprises two antigen binding sites (1410 and 1415), and a portion of the two dark gray scaffolds contain Fc domains (1440 and 1445). The immune-stimulatory compounds (1430 and 1440) are conjugated to the antibody construct by linkers (1460 and 1470).

FIG. 15 illustrates a schematic of an exemplary conjugate. An antibody construct contains two scaffolds as shown in light gray and two scaffolds as shown in dark gray. The antibody construct comprises two antigen binding sites (1510 and 1515), and a portion of the two dark gray scaffolds contain Fc domains (1540 and 1545). The immune-stimulatory compounds (1530 and 1540) are conjugated to the antibody construct by linkers (1560 and 1570). Targeting binding domains are conjugated to the antibody construct (1580 and 1585).

FIG. 16 is the two-dimensional structure of the heavy chain of dacetuzumab.

FIG. 17 is the two-dimensional structure of the light chain of dacetuzumab.

FIG. 18 is the two-dimensional structure of the heavy chain of bleselumab.

FIG. 19 is the two-dimensional structure of the light chain of bleselumab.

FIG. 20 is the two-dimensional structure of the heavy chain of lucatumumab.

FIG. 21 is the two-dimensional structure of the light chain of lucatumumab

FIG. 22 is the two-dimensional structure of the heavy chain of ADC-1013.

FIG. 23 is the two-dimensional structure of the light chain of ADC-1013.

FIG. 24 is the two-dimensional structure of the heavy chain of humanized rabbit antibody APX005.

FIG. 25 is the two-dimensional structure of the light chain of humanized rabbit antibody APX005.

FIG. 26 is the two-dimensional structure of the heavy chain of Chi Lob 7/4.

FIG. 27 is the two-dimensional structure of the light chain of Chi Lob 7/4.

FIG. 28 shows HPLC analysis of SBT-040-G1WT conjugated to a Cys-targeted drug linker tool compound.

FIG. 29 shows HPLC analysis of SBT-040-G1WT conjugated to ATAC2.

FIG. 30 shows HPLC analysis of SBT-040-G2WT conjugated to ATAC2.

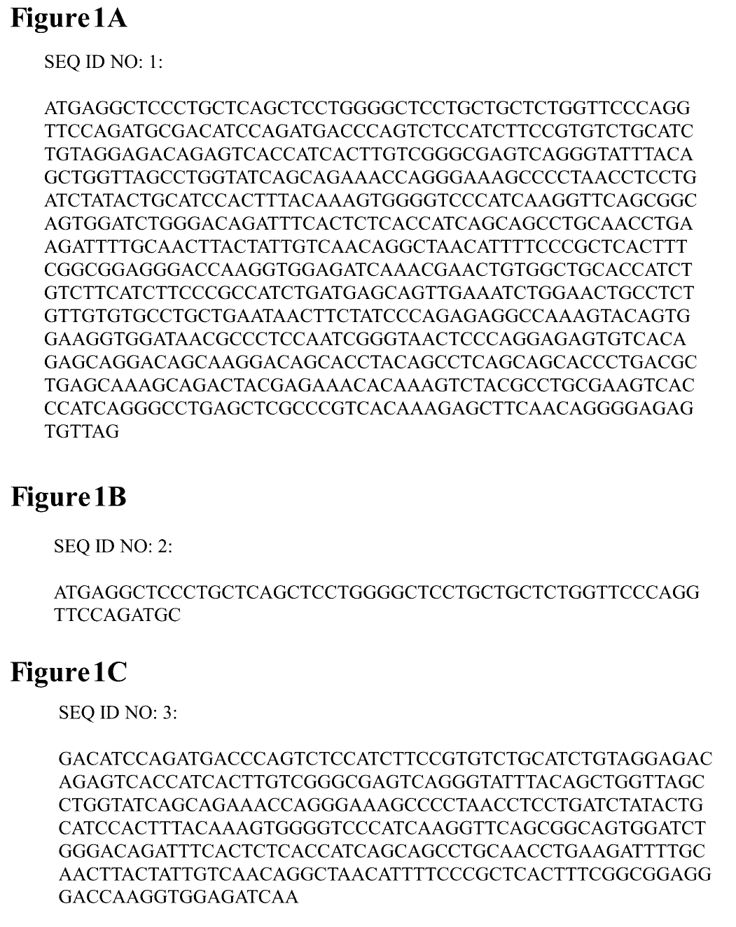

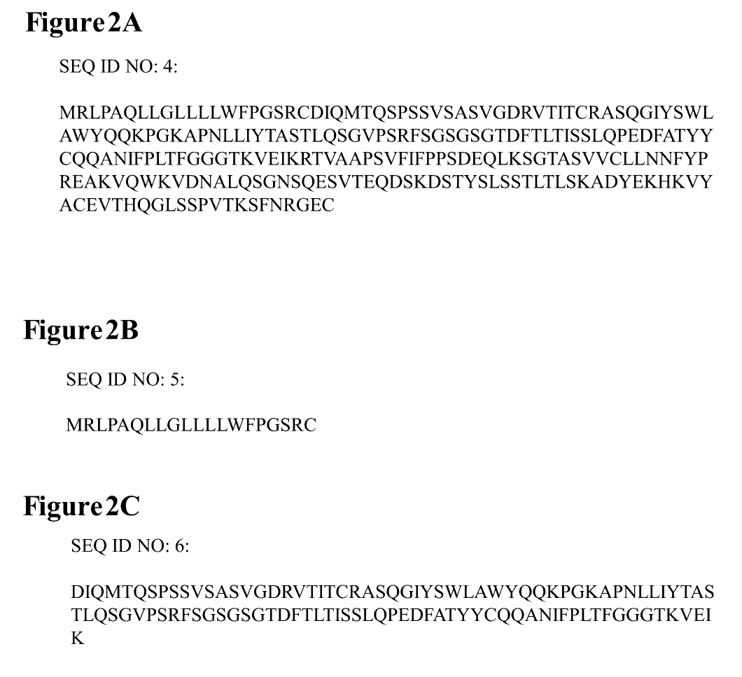

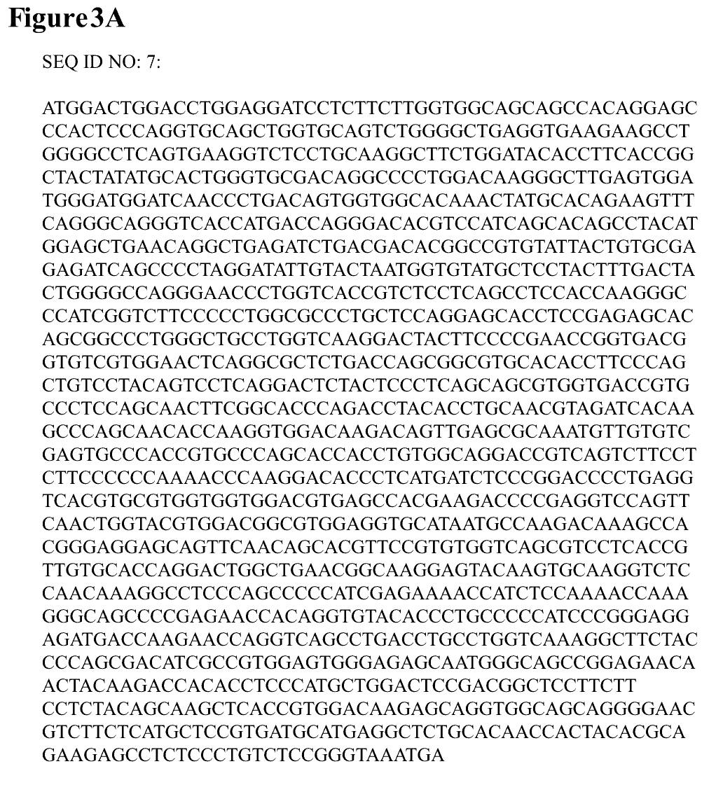

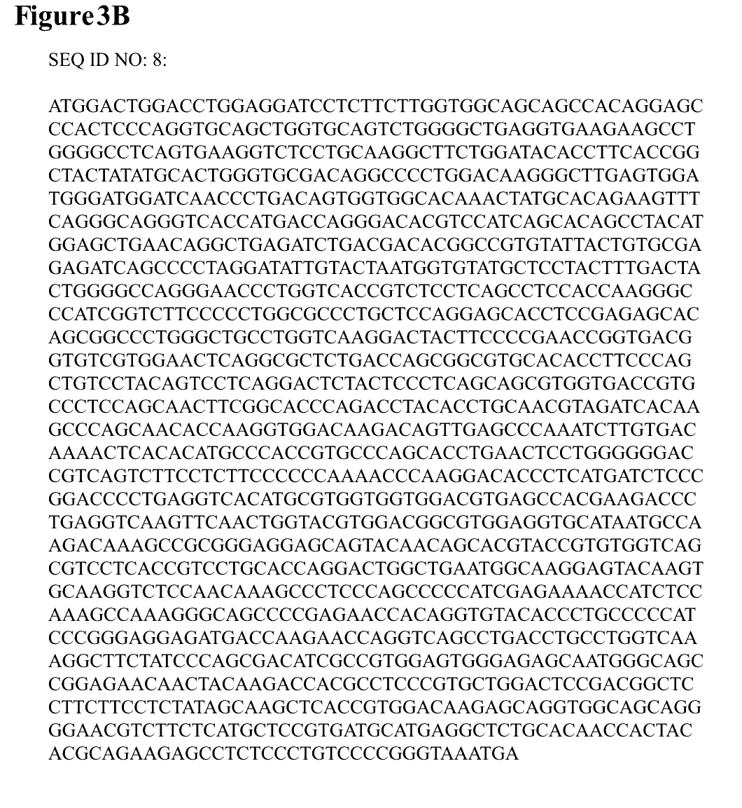

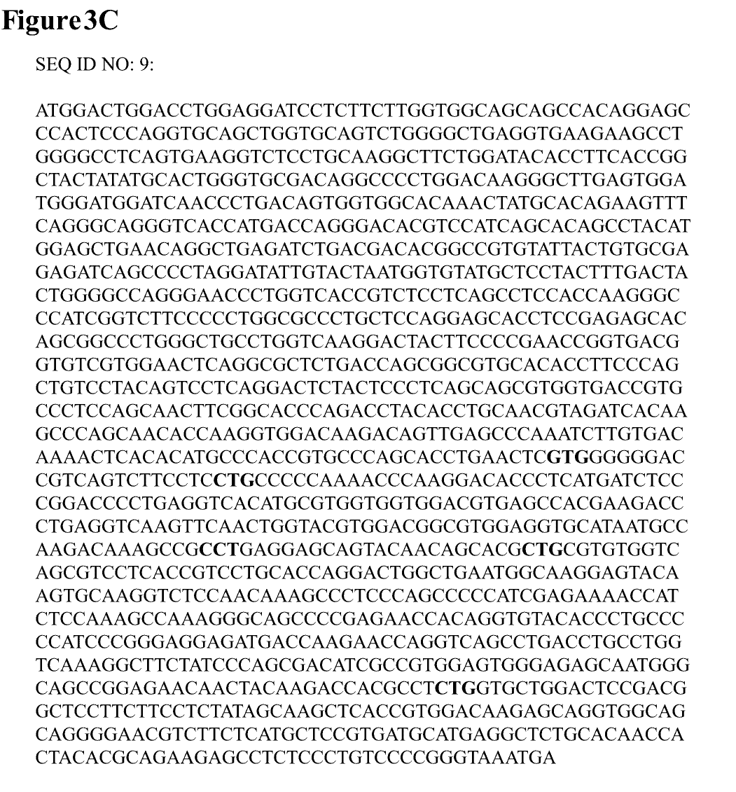

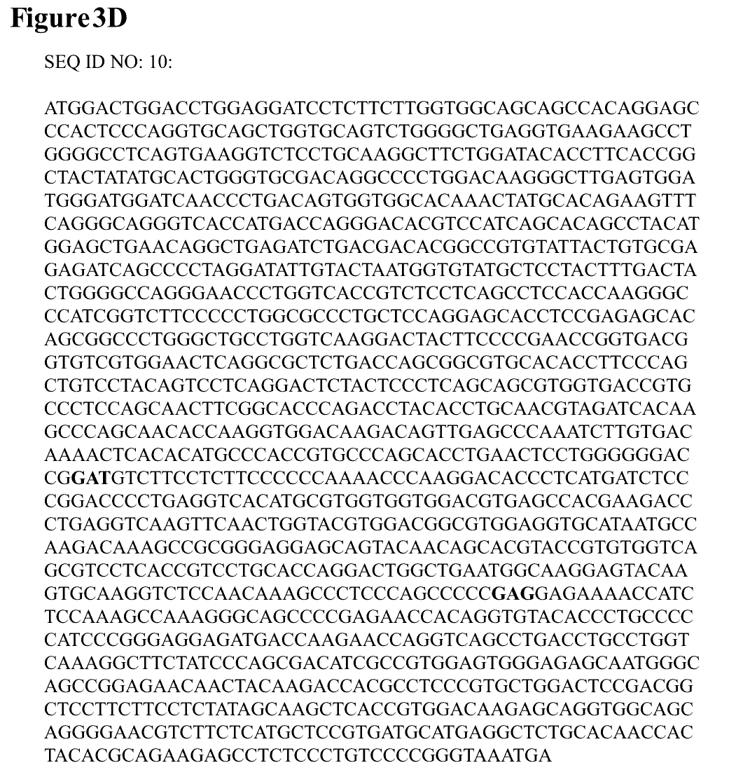

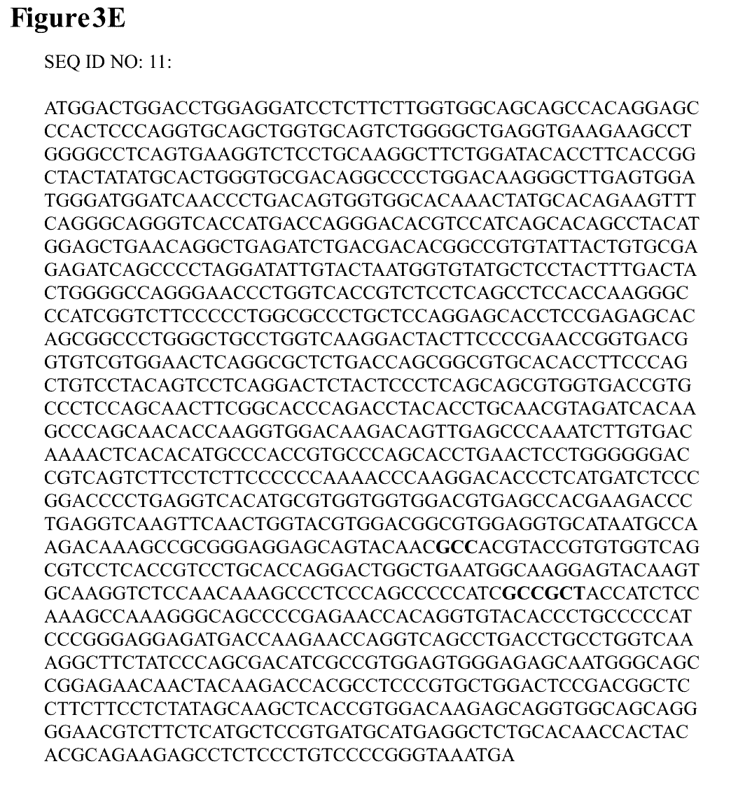

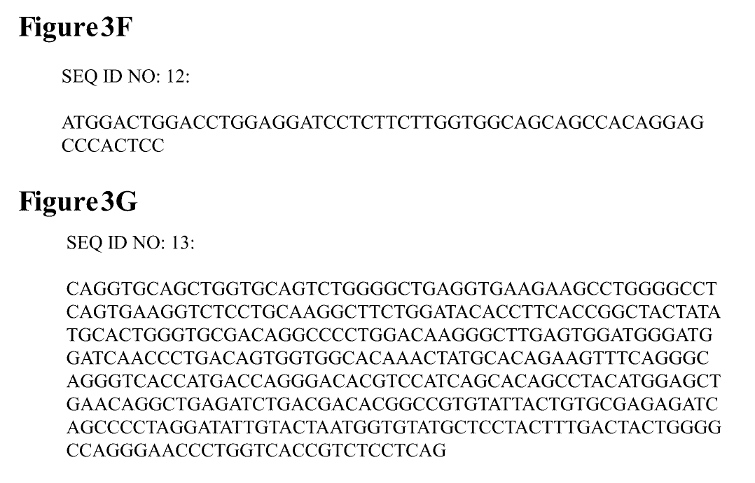

### DETAILED DESCRIPTION

Additional aspects and advantages of the present disclosure will become apparent to those skilled in this art from the following detailed description, wherein illustrative aspects of the present disclosure are shown and described. As will be realized, the present disclosure is capable of other and different aspects, and its several details are capable of modifications in various respects, all without departing from the disclosure. Accordingly, the drawings and description are to be regarded as illustrative in nature, and not as restrictive.

Cancer is one of the leading causes of death in the United States. Conventional methods of cancer treatment like chemotherapy, surgery or radiation therapy, can be limited in their efficacy since they are often nonspecific to the cancer. In many cases tumors, however, can specifically express genes whose products are required for inducing or maintaining the malignant state. These proteins may serve as antigen markers for the development and establishment of efficient anti-cancer treatments.

Antigens can elicit an immune response. These antigens can be either proteins, polysaccharides, lipids, or glycolipids, which can be recognized as “foreign” by immune cells, such as T cells and B cells. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

In cancer, there are four general groups of tumor antigens: (i) viral tumor antigens which can be identical for any viral tumor of this type, (ii) carcinogenic tumor antigens which can be specific for patients and for the tumors, (iii) isoantigens of the transplantation type or tumor-specific transplantation antigens which can be different in all individual types of tumor but can be the same in different tumors caused by the same virus; and (iv) embryonic antigens.

As a result of the discovery of tumor antigens, tumor antigens have become important in the development of new cancer treatments that can specifically target the cancer. This has led to the development of antibodies directed against these tumor antigens.

In addition to the development of antibodies against tumor antigens for cancer treatment, antibodies that target immune cells to boost the immune response have also been developed. For example, an anti-CD40 antibody that is a CD40 agonist can be used to activate dendritic cells to enhance the immune response.

Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-1βp, IFN-γ, GM-CSF, and LPS induced signaling events.

Humoral and cellular immune responses can be regulated, in part, by CD40. For example, in the absence of CD40 activation by its cognate binding partner, CD40 Ligand (CD40L), antigen presentation can result in tolerance. However, CD40 activation can ameliorate tolerance. In addition, CD40 activation can positively impact immune responses by enhancing antigen presentation by antigen presenting cells (APC), increasing cytokine and chemokine secretion, stimulating expression of and signaling by co-stimulatory molecules, and activating the cytolytic activity of different types of immune cells. Accordingly, the interaction between CD40 and CD40L can be essential to maintain proper humoral and cellular immune responses.

The intracellular effects of CD40 and CD40L interaction can include association of the CD40 cytoplasmic domain with TRAFs (TNF-R associated factors), which can lead to the activation of NFκB and Jun/AP1 pathways. While the response to activation of NFκB and Jun/AP1 pathways can be cell type-specific, often such activation can lead to increased production and secretion of cytokines, including IL-6, IL-8, IL-12, IL-15; increased production and secretion of chemokines, including MIP1α and β and RANTES; and increased expression of cellular adhesion molecules, including ICAM. While the effects of cytokines, chemokines and cellular adhesion molecules can be widespread, such effects can include enhanced survival and activation of T cells.

In addition to the enhanced immune responses induced by CD40 activation, CD40 activation can also be involved in chemokine- and cytokine-mediated cellular migration and differentiation; activation of immune cells, including monocytes; activation of and increased cytolytic activity of immune cells, including cytolytic T lymphocytes and natural killer cells; induction of CD40-positive tumor cell apoptosis and enhanced immunogenicity of CD40-positive tumors. In addition, CD40 can initiate and enhance immune responses by many different mechanisms, including, inducing antigen-presenting cell maturation and increased expression of costimulatory molecules, increasing production of and secretion of cytokines, and enhancing effector functions.

CD40 activation can be effective for inducing immune-mediated antitumor responses. For example, CD40 activation reverses host immune tolerance to tumor-specific antigens which leads to enhanced antitumor responses by T cells. Such antitumor activity can also occur in the absence of immune cells. Similarly, antitumor effects can occur in response to anti-CD40 antibody-mediated activation of CD40 and can be independent of antibody-dependent cellular cytotoxicity. In addition to other CD40-mediated mechanisms of antitumor effects, CD40L-stimulation can cause dendritic cell maturation and stimulation. CD40L-stimulated dendritic cells can contribute to the antitumor response. Furthermore, vaccination strategies including CD40 can result in regression of CD40-positive and CD40-negative tumors.

CD40 activating antibodies (e.g., anti-CD40 activating monoclonal antibodies) can be useful for treatment of tumors. This can occur through one or more mechanisms, including cell activation, antigen presentation, production of cytokines and chemokines, amongst others. For example, CD40 antibodies activate dendritic cells, leading to processing and presentation of tumor antigens as well as enhanced immunogenicity of CD40-positive tumor cells. Not only can enhanced immunogenicity result in activation of CD40-positive tumor specific CD4⁺ and CD8⁺ T cells, but further antitumor activity can include, recruitment and activation monocytes, enhanced cytolytic activity of cytotoxic lymphocytes and natural killer cells as well as induction of apoptosis or by stimulation of a humoral response so as to directly target tumor cells. In addition, tumor cell debris, including tumor-specific antigens, can be presented to other cells of the immune system by CD40-activated antigen presenting cells.

Since CD40 can be important in an immune response, there is a need for enhanced CD40 meditated signaling events to provide reliable and rapid treatment options to patients suffering from diseases which may be ameliorated by treatment with CD40-targeted therapeutic strategies.

The HER2/neu (human epidermal growth factor receptor 2/receptor tyrosine-protein kinase erbB-2) is part of the human epidermal growth factor family. Overexpression of this protein has shown to play an important role in the progression of cancer, for example, breast cancer. The HER2/neu protein functions as a receptor tyrosine kinase and autophosphorylates upon dimerization with binding partners. HER2/neu can activate several signaling pathways including, for example, mitogen-activated protein kinase, phosphoinositide 3-kinase, phospholipase Cγ, protein kinase C, and signal transducer and activator of transcription (STAT). Several compounds have been developed to inhibit HER2/neu including for example, the monoclonal antibody trastuzumab and the monoclonal antibody pertuzumab.

Immune-stimulatory molecular motifs, such as Pathogen-Associated Molecular Pattern molecules, (PAMPs) can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens Similar to other protein families, TLRs can have many isoforms, including TLR4, TLR7 and TLR8. Several agonists targeting activation of different TLRs can be used in various immunotherapies, including vaccine adjuvants and in cancer immunotherapies. TLR agonists can be synthetic or biosynthetic agonists. TLR agonists can also be PAMPs. Additional immune-stimulatory compounds, such as cytosolic DNA and unique bacterial nucleic acids called cyclic dinucleotides, can be recognized by Interferon Regulatory Factor (IRF) or stimulator of interferon genes (STING), which can act a cytosolic DNA sensor. ADU-S100 can be a STING agonist. Non-limiting examples of STING agonists include:

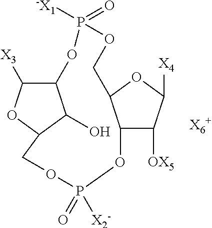

wherein in some embodiments, X₁═X₂═O; X₃=G; X₄=G; X₅═CO(CH₂)₁₂CH₃; X₆=2 TEAH; in some embodiments, X₁═X₂═S [Rₚ,Rₚ]; X₃=G; X₄=A; X₅═H; X₆=2 TEAH; in some embodiments, X₁═X₂═S [Rₚ,Rₚ]; X₃=A; X₄=A; X₅═H; X₆=2 Na; in some embodiments, X₁═X₂═S [Rₚ,Rₚ]; X₃=A; X₄=A; X₅═H; X₆=2 NH₄; and in some embodiments, X₁═X₂═O; X₃=G; X₄=A; X₅═H; X₆=2 TEAH,

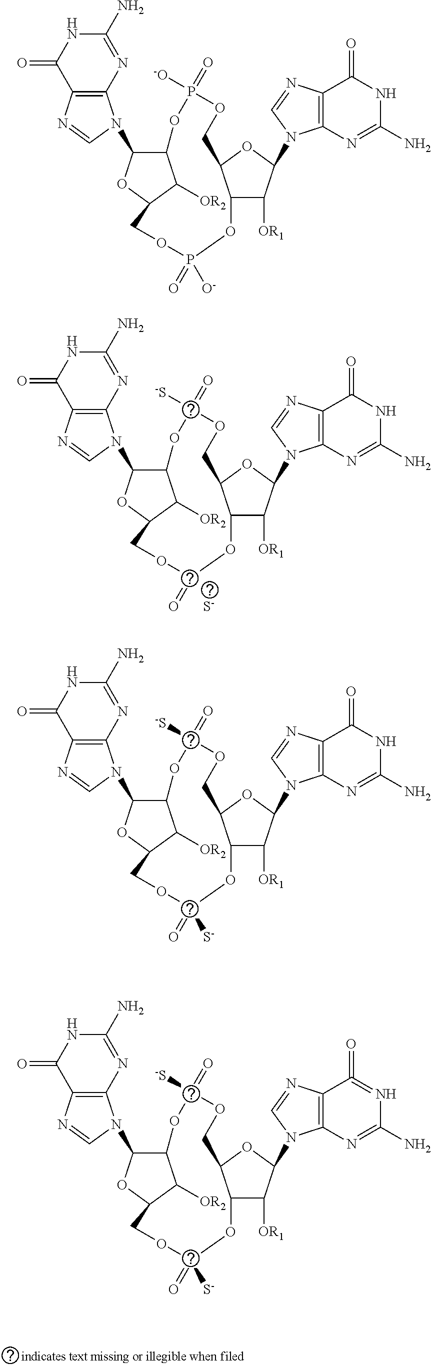

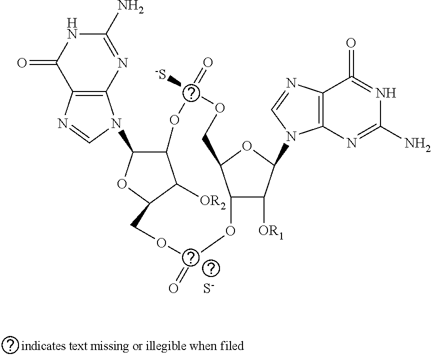

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

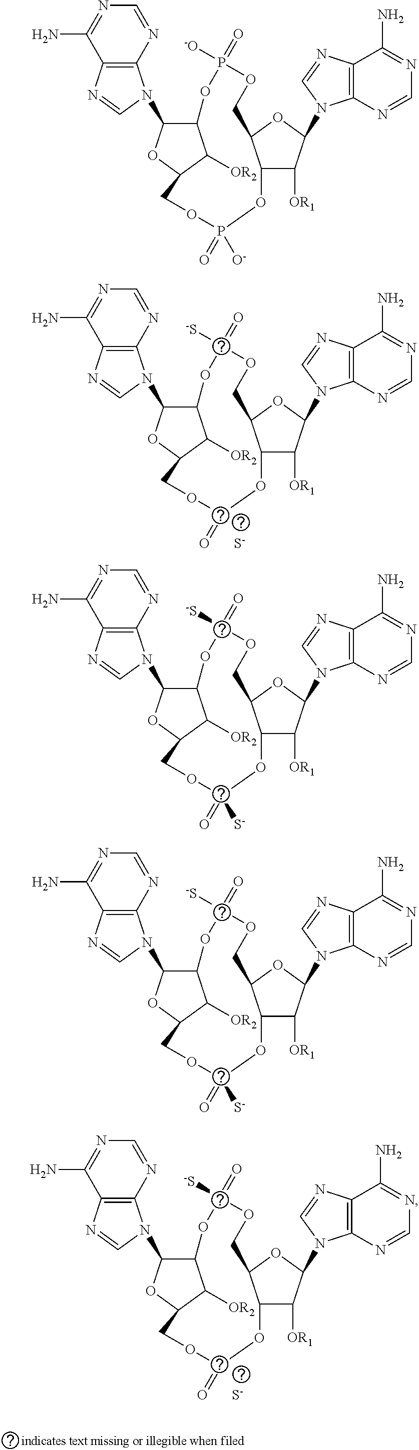

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

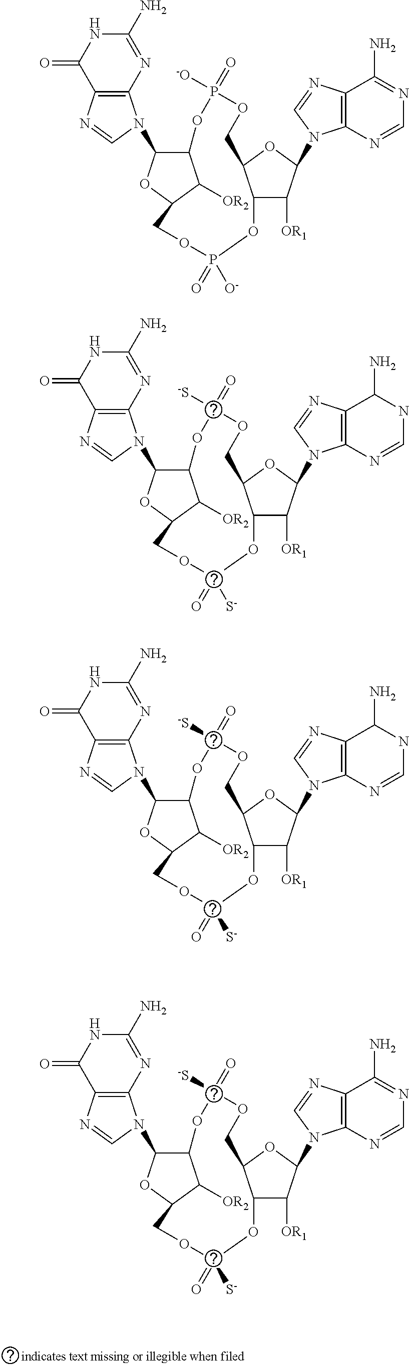

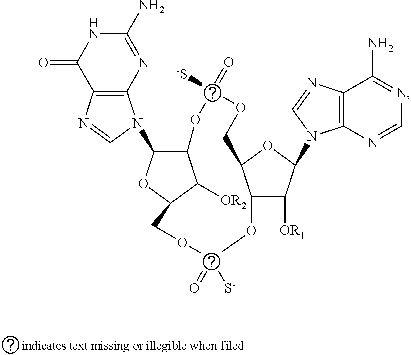

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

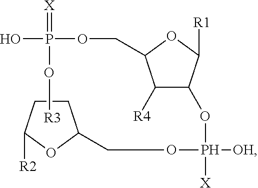

wherein each X is independently O or S, and R3 and R4 are each independently H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ where R′ is H or lower alkyl, —CH₂OH, and —CONH₂, wherein R3 and R4 are not both H,

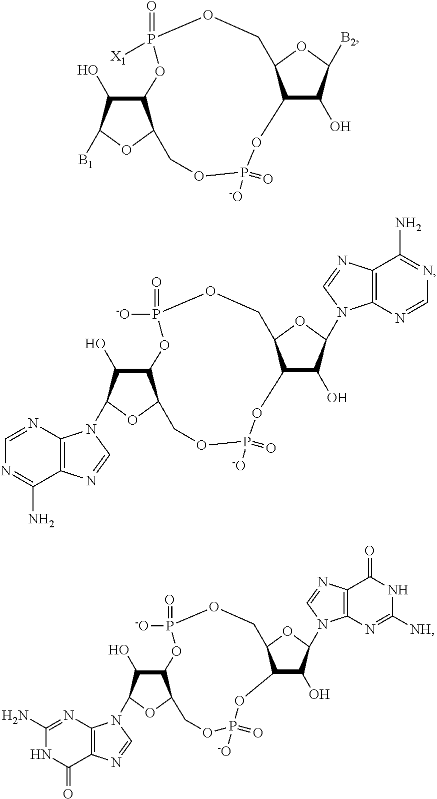

wherein X₁═X₂═O; X₁═X₂═S; or X₁═O and X₂═S,

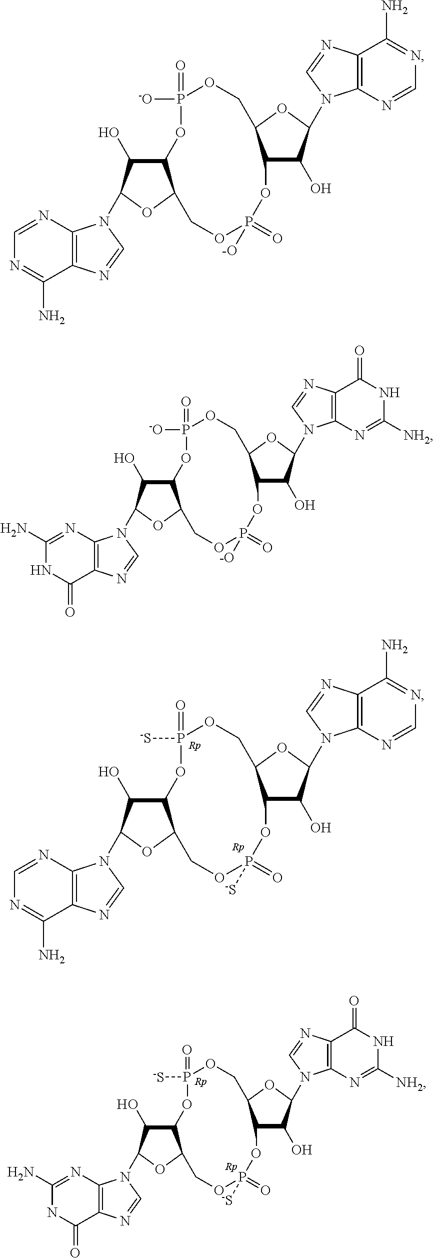

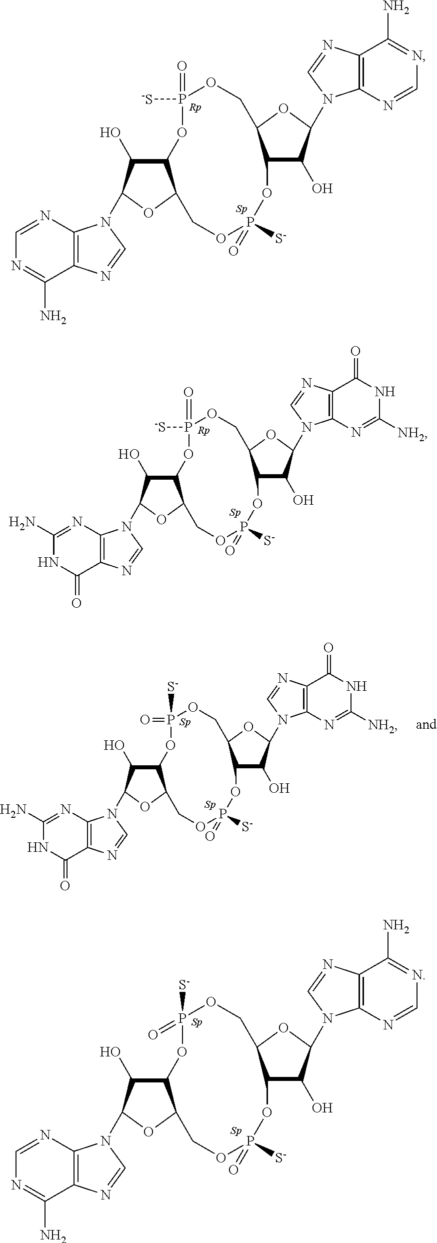

KIN700, KIN1148, KIN600, KIN500, KIN100, KIN101, KIN400, KIN2000, or SB-9200 can be recognized by Interferon Regulatory Factor (IRF), which can play a role in immunoregulation by TLRs and other pattern recognition receptors.

TLR agonists can range from simple molecules to complex macromolecules. Likewise, the sizes of TLR agonists can range from small to large. For example, small molecule TLR agonists can include S-27609, CL307, UC-IV150, imiquimod, gardiquimod, resiquimod, motolimod, VTX-1463, GS-9620, GSK2245035, TMX-101, TMX-201, TMX-202, isatoribine, AZD8848, MEDI9197, 3M-051, 3M-852, 3M-052, 3M-854A, S-34240, CL663, KIN1148, KU34B, SB-9200, and analogues of adenosine and guanosine Similarly, large macromolecules can include lipopolysaccharide (LPS) and nucleic acids elements such as CpG and polyI:C.

Imiquimod, a synthetic TLR7 agonist, is currently approved for human therapeutic applications. Contained in a cream and marketed under the brand name Aldara, imiquimod serves as a topical treatment for a variety of indications with immune components, such as, actinic keratosis, genital warts, and basal cell carcinomas. In addition, imiquimod is indicated as a candidate adjuvant for enhancing adaptive immune responses when applied topically at an immunization site.

Another type of immune stimulatory molecular motif, damage-associated molecular pattern molecules (DAMPs), can initiate and maintain an immune response occurring as part of the non-infectious inflammatory response. DAMPs can be specially localized proteins that, when detected by the immune system in a location other than where DAMPs should be located, activate the immune system. Often, DAMPs can be nuclear or cytosolic proteins and upon release from the nucleus or cytosol, DAMP proteins can become denatured through oxidation. Examples of DAMP proteins can include chromatin-associated protein high-mobility group box 1 (HMGB1), S100 molecules of the calcium modulated family of proteins and glycans, such as hyaluronan fragments, and glycan conjugates. DAMPs can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

Therapeutic application of DAMPs can focus on indications with an immune component, such as arthritis, cancer, ischemia-reperfusion injury, myocardial infarction and stroke. In these indications, the mechanism of action for DAMP therapeutic effects can include the prevention of DAMP release using therapeutic strategies, such as proapoptotic interventions, platinums and ethyl pyruvate, extracellular neutralization or blockade of DAMP release or signaling using therapeutic strategies such as anti-HMGB1, rasburiaspect and sRAGE, as well as direct or indirect blockade of DAMP receptors, and downstream signaling events, using therapeutic strategies such as RAGE small molecule antagonists; TLR4 antagonists and antibodies to DAMP-R.

Additionally, the immune response elicited by TLR agonists can further be enhanced when co-administered with a CD40-agonist antibody. For example, co-administration of a TLR agonist such as poly IC:LC with a CD40-agonist antibody can synergize to stimulate a greater CD8⁺ T cell response than either agonist alone.

However, therapeutic use of PAMPs and DAMPs or other mechanisms of intervention can be limited because systemic activation of PAMP and DAMP signaling pathways can have life-threatening consequences due to cytokine syndrome-induced or cytokine storm-induced toxic shock syndrome. Accordingly, there is a critical need for therapeutic, clinically relevant targeted delivery of PAMP and DAMP agonists for safe and effective strategies to enhance immune responses. The presently described conjugate can be utilized as a safe and effective strategy to enhance immune responses. A conjugate can comprise an antibody construct and an immune-stimulatory compound.

### Antibody Construct

An antibody construct can contain an antigen binding domain. An antigen binding domain can be a domain that can specifically bind to an antigen. An antigen binding domain can be an antigen-binding portion of an antibody or an antibody fragment. An antigen binding domain can be one or more fragments of an antibody that can retain the ability to specifically bind to an antigen. An antigen binding domain can be any antigen binding fragment. An antigen binding domain can recognize a single antigen. An antigen binding domain can recognize two or more antigens. An antibody construct can contain two antigen binding domains. An antibody construct can contain two antigen binding domains in which each antigen binding domain can recognize the same antigen. An antibody construct can contain two antigen binding domains in which each antigen binding domain can recognize different antigens. An antigen binding domain can be in a scaffold, in which a scaffold is a supporting framework for the antigen binding domain. An antigen binding domain can be in a non-antibody scaffold. An antigen binding domain can be in an antibody scaffold. An antibody construct can comprise an antigen binding domain in a scaffold.

The antigen binding domain of an antibody construct can be selected from any domain that binds the antigen including, but not limited to, from a monoclonal antibody, a polyclonal antibody, a recombinant antibody, or a functional fragment thereof, for example, a heavy chain variable domain (V_{H}) and a light chain variable domain (V_{L}), a DARPin, an affimer, an avimer, a knottin, a monobody, or an affinity clamp. The antigen binding domain of an antibody construct can be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a DARPin, an affimer, an avimer, a knottin, a monobody, or an affinity clamp.

An antigen binding domain of an antibody construct, for example an antigen binding domain from a monoclonal antibody, can comprise a light chain and a heavy chain. In one aspect, the monoclonal antibody binds to CD40 and comprises the light chain of an anti-CD40 antibody and the heavy chain of an anti-CD40 antibody, which bind a CD40 antigen. In another aspect, the monoclonal antibody binds to a tumor antigen comprises the light chain of a tumor antigen antibody and the heavy chain of a tumor antigen antibody, which bind the tumor antigen.

An antibody construct can be an antibody. An antibody molecule can consist of two identical light protein chains and two identical heavy protein chains, all held together covalently by precisely located disulfide linkages. The N-terminal regions of the light and heavy chains together can form the antigen recognition site of each antibody. Structurally, various functions of an antibody can be confined to discrete protein domains (i.e., regions). The sites that can recognize and can bind antigen consist of three complementarity determining regions (CDRs) that can lie within the variable heavy chain regions and variable light chain regions at the N-terminal ends of the two heavy and two light chains. The constant domains can provide the general framework of the antibody and may not be involved directly in binding the antibody to an antigen, but can be involved in various effector functions, such as participation of the antibody in antibody-dependent cellular cytotoxicity.

The domains of natural light and heavy chains can have the same general structures, and each domain can comprise four framework regions, whose sequences can be somewhat conserved, connected by three hyper-variable regions or CDRs. The four framework regions can largely adopt a β-sheet conformation and the CDRs can form loops connecting, and in some aspects forming part of, the β-sheet structure. The CDRs in each chain can be held in close proximity by the framework regions and, with the CDRs from the other chain, can contribute to the formation of the antigen binding site.

An antibody of an antibody construct can include an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, γ, ε, γ, and μ, respectively. The light chains can be one of either kappa or κ and lambda or λ, based on the amino acid sequences of the constant domains. The Fc region can contain an Fc domain. An Fc receptor can bind an Fc domain. Antibody constructs can also include any fragment or recombinant forms thereof, including but not limited to scFvs, ‘T-bodies’, anti-calins, centyrins, affibodies, domain antibodies, or peptibodies.

An antibody can comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. Examples of antibody binding domains can include, but are not limited to, Fab, variable Fv fragment and other fragments, combinations of fragments or types of fragments known or knowable to one of ordinary skill in the art.

An antibody construct can comprise an antigen binding domain of an antibody. An antigen binding domain of an antibody can comprise one or more light chain (LC) CDRs and one or more heavy chain (HC) CDRs, one or more LC CDRs or one or more HC CDRs. For example, an antibody binding domain of an antibody can comprise one or more of the following: a light chain complementary determining region 1 (LC CDR1), a light chain complementary determining region 2 (LC CDR2), or a light chain complementary determining region 3 (LC CDR3). For another example, an antibody binding domain can comprise one or more of the following: a heavy chain complementary determining region 1 (HC CDR1), a heavy chain complementary determining region 2 (HC CDR2), or a heavy chain complementary determining region 3 (HC CDR3).

An antibody construct can comprise an antibody fragment. An antibody fragment can include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, can be coded for by separate genes, they can be linked by a synthetic linker to be made as a single protein chain in which the V_{L} and V_{H} regions pair to form monovalent molecules.

F(ab′)2 and Fab′ moieties can be produced by treating immunoglobulin (monoclonal antibody) with a protease such as pepsin and papain, and can include an antibody fragment generated by digesting immunoglobulin near the disulfide bonds existing between the hinge regions in each of the two H chains. The Fab fragment can also contain the constant domain of the light chain and the first constant domain (C_{H1}) of the heavy chain. Fab′ fragments can differ from Fab fragments by the addition of a few residues at the carboxyl terminus of the heavy chain C_{H1} domain including one or more cysteine(s) from the antibody hinge region.

An Fv can be the minimum antibody fragment which contains a complete antigen-recognition and antigen-binding site. This region can consist of a dimer of one heavy chain and one light chain variable domain in tight, non-covalent association. In this configuration the three hypervariable regions of each variable domain can interact to define an antigen-binding site on the surface of the V_{H}-V_{L} dimer. A single variable domain (or half of an Fv comprising only three hypervariable regions specific for an antigen) can recognize and bind antigen, although at a lower affinity than the entire binding site.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

An antibody construct can comprise an anti-CD40 antibody. An antibody construct can comprise an antibody light chain. A light chain can be a light chain of an anti-CD40 antibody which can bind a CD40 antigen. A light chain of an anti-CD40 antibody can be expressed from a DNA sequence comprising ATGAGGCTCCCTGCTCAGCTCCTGGGGCTCCTGCTGCTCTGGTTCCCAGGTTCCAGATGC GACATCCAGATGACCCAGTCTCCATCTTCCGTGTCTGCATCTGTAGGAGACAGAGTCAC CATCACTTGTCGGGCGAGTCAGGGTATTTACAGCTGGTTAGCCTGGTATCAGCAGAAAC CAGGGAAAGCCCCTAACCTCCTGATCTATACTGCATCCACTTTACAAAGTGGGGTCCCA TCAAGGTTCAGCGGCAGTGGATCTGGGACAGATTTCACTCTCACCATCAGCAGCCTGCA ACCTGAAGATTTTGCAACTTACTATTGTCAACAGGCTAACATTTTCCCGCTCACTTTCGG CGGAGGGACCAAGGTGGAGATCAAACGAACTGTGGCTGCACCATCTGTCTTCATCTTCC CGCCATCTGATGAGCAGTTGAAATCTGGAACTGCCTCTGTTGTGTGCCTGCTGAATAAC TTCTATCCCAGAGAGGCCAAAGTACAGTGGAAGGTGGATAACGCCCTCCAATCGGGTA ACTCCCAGGAGAGTGTCACAGAGCAGGACAGCAAGGACAGCACCTACAGCCTCAGCAG CACCCTGACGCTGAGCAAAGCAGACTACGAGAAACACAAAGTCTACGCCTGCGAAGTC ACCCATCAGGGCCTGAGCTCGCCCGTCACAAAGAGCTTCAACAGGGGAGAGTGTTAG (SEQ ID NO: 1). A light chain of an anti-CD40 antibody can be expressed from DNA sequence comprising greater than 70%, greater than 75%, greater than 80%, greater than 85%, greater than 90%, greater than 95% or greater than 99% homology to SEQ ID NO: 1. A variable region of a light chain of an anti-CD40 antibody can be expressed from a DNA sequence comprising GACATCCAGATGACCCAGTCTCCATCTTCCGTGTCTGCATCTGTAGGAGACAGAGTCAC CATCACTTGTCGGGCGAGTCAGGGTATTTACAGCTGGTTAGCCTGGTATCAGCAGAAAC CAGGGAAAGCCCCTAACCTCCTGATCTATACTGCATCCACTTTACAAAGTGGGGTCCCA TCAAGGTTCAGCGGCAGTGGATCTGGGACAGATTTCACTCTCACCATCAGCAGCCTGCA ACCTGAAGATTTTGCAACTTACTATTGTCAACAGGCTAACATTTTCCCGCTCACTTTCGG CGGAGGGACCAAGGTGGAGATCAA (SEQ ID NO: 3). A variable region of a light chain of an anti-CD40 antibody can be expressed from a DNA sequence comprising greater than 70%, greater than 75%, greater than 80%, greater than 85%, greater than 90%, greater than 95% or greater than 99% homology to SEQ ID NO: 3. Additionally, anti-CD40 antibodies expressed from SEQ ID NO: 1, or expressed from a DNA sequence comprising greater than 70% homology to SEQ ID NO: 1 can have a dissociation constant (K_{d}) for CD40 that is less than 10 nM. Anti-CD40 antibodies expressed from SEQ ID NO: 1, or expressed from a DNA sequence comprising greater than 70% homology to SEQ ID NO: 1 can have a dissociation constant (K_{d}) for CD40 that is less than 1 nM, less than 100 pM, less than 10 pM, less than 1 pM, or less than 0.1 pM. The anti-CD40 light chain can be expressed with any anti-CD40 heavy chain or fragment thereof. The anti-CD40 light chain can also expressed with any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct.

A light chain of an anti-CD40 antibody can comprise an amino acid sequence MRLPAQLLGLLLLWFPGSRCDIQMTQSPSSVSASVGDRVTITCRASQGIYSWLAWYQQKPG KAPNLLIYTASTLQSGVPSRFSGSGSGTDFTLTISSLQPEDFATYYCQQANIFPLTFGGGTKVE IKRTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 4). A light chain of an anti-CD40 antibody can comprise an amino sequence with greater than 70%, greater than 75%, greater than 80%, greater than 85%, greater than 90%, greater than 95% or greater than 99% homology to SEQ ID NO: 4. A variable region of a light chain of an anti-CD40 antibody can comprise an amino acid sequence DIQMTQSPSSVSASVGDRVTITCRASQGIYSWLAWYQQKPGKAPNLLIYTASTLQSGVPSRF SGSGSGTDFTLTISSLQPEDFATYYCQQANIFPLTFGGGTKVEIK (SEQ ID NO: 6). A variable region of a light chain of an anti-CD40 antibody can comprise an amino acid sequence with greater than 70%, greater than 75%, greater than 80%, greater than 85%, greater than 90%, greater than 95% or greater than 99% homology to SEQ ID NO: 6. Additionally, anti-CD40 antibodies comprising SEQ ID NO: 4, or comprising an amino acid sequence with greater than 70% homology to SEQ ID NO: 4 can have a dissociation constant (K_{d}) for CD40 that is less than 10 nM. Anti-CD40 antibodies comprising SEQ ID NO: 4, or comprising an amino acid sequence with greater than 70% homology to SEQ ID NO: 4 can have a dissociation constant (K_{d}) for CD40 that is less than 1 nM, less than 100 pM, less than 10 pM, less than 1 pM, or less than 0.1 pM. The anti-CD40 light chain can be purified, and can be combined with a pharmaceutically acceptable carrier. The anti-CD40 light chain can be combined with any anti-CD40 heavy chain or fragment thereof. The anti-CD40 light chain can also be combined any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to anti-CD40 antibodies created for use in the veterinary sciences and/or in laboratory animals.

An antibody construct can comprise an antibody light chain. A light chain can be a light chain of an anti-CD40 antibody which can bind a CD40 antigen. A light chain of an anti-CD40 antibody can be SBT-040 VL-Ck. SBT-040 VL-Ck can comprise an amino acid sequence with greater than 70%, greater than 75%, greater than 80%, greater than 85%, greater than 90%, greater than 95% or greater than 99% homology to SEQ ID NO: 26.

A light chain of an anti-CD40 antibody can comprise a CDR. A light chain of an anti-CD40 antibody can comprise a CDR with SEQ ID NO: 27. A light chain of an anti-CD40 antibody can comprise a CDR with SEQ ID NO: 28. A light chain of an anti-CD40 antibody can comprise a CDR with SEQ ID NO: 29. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 27. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 28. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 29.

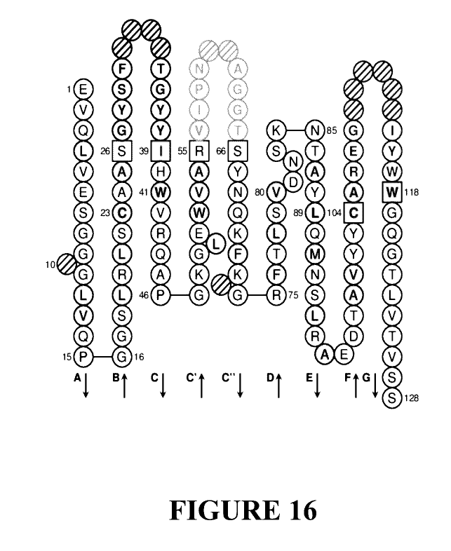

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be dacetuzumab. Dacetuzumab can comprise an amino acid sequence with greater than 70%, greater than 75%, greater than 80%, greater than 85%, greater than 90%, greater than 95% or greater than 99% homology to SEQ ID NO: 38. A heavy chain of an anti-CD40 antibody can comprise a CDR. A heavy chain of an anti-CD40 antibody can comprise a CDR with SEQ ID NO: 39. A heavy chain of an anti-CD40 antibody can comprise a CDR with SEQ ID NO: 40. A heavy chain of an anti-CD40 antibody can comprise a CDR with SEQ ID NO: 41. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 39. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 40. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 41. The two-dimensional structure of the dacetuzumab heavy chain is shown in FIG. 16.

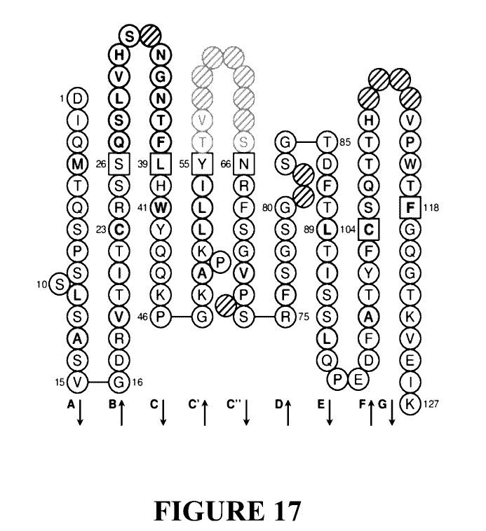

An antibody construct can comprise an antibody light chain. A light chain can be a light chain of an anti-CD40 antibody which can bind a CD40 antigen. A light chain of an anti-CD40 antibody can be dacetuzumab. Dacetuzumab can comprise an amino acid sequence with greater than 70%, greater than 75%, greater than 80%, greater than 85%, greater than 90%, greater than 95% or greater than 99% homology to SEQ ID NO: 42. A light chain of an anti-CD40 antibody can comprise a CDR. A light chain of an anti-CD40 antibody can comprise a CDR with SEQ ID NO: 43. A light chain of an anti-CD40 antibody can comprise a CDR with SEQ ID NO: 44. A light chain of an anti-CD40 antibody can comprise a CDR with SEQ ID NO: 45. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 43. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 44. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 45. The two-dimensional structure of the dacetuzumab light chain is shown in FIG. 17.

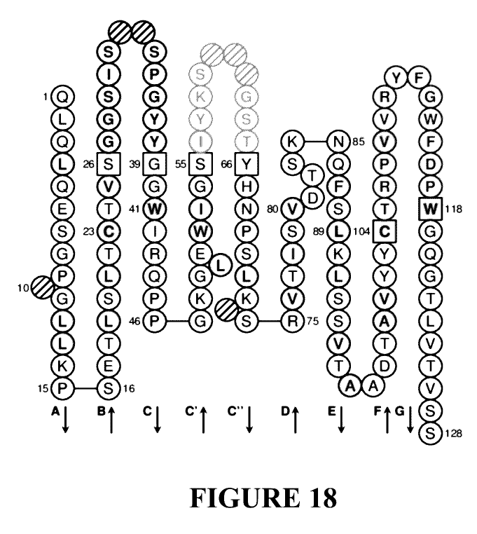

An antibody construct can comprise an antibody heavy chain. A heavy chain can be a heavy chain of an anti-CD40 antibody which can bind a CD40 antigen. A heavy chain of an anti-CD40 antibody can be an IgG4 isotype. A heavy chain of an anti-CD40 antibody can be bleselumab. Bleselumab can comprise an amino acid sequence with greater than 70%, greater than 75%, greater than 80%, greater than 85%, greater than 90%, greater than 95% or greater than 99% homology to SEQ ID NO: 46. A heavy chain of an anti-CD40 antibody can comprise a CDR. A heavy chain of an anti-CD40 antibody can comprise a CDR with SEQ ID NO: 47. A heavy chain of an anti-CD40 antibody can comprise a CDR with SEQ ID NO: 48. A heavy chain of an anti-CD40 antibody can comprise a CDR with SEQ ID NO: 49. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 47. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 48. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 49. The two-dimensional structure of the bleselumab heavy chain is shown in FIG. 18.

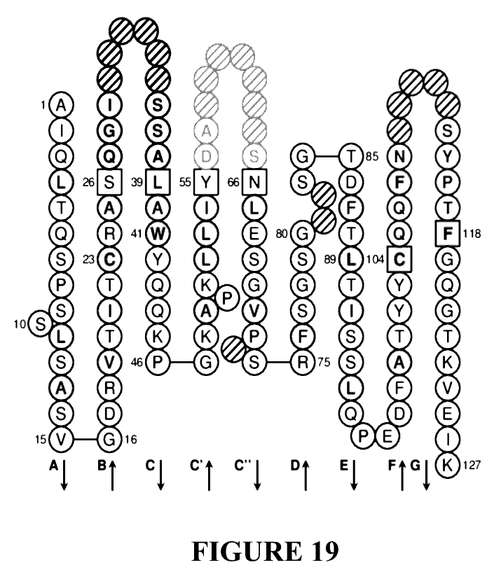

An antibody construct can comprise an antibody light chain. A light chain can be a light chain of an anti-CD40 antibody which can bind a CD40 antigen. A light chain of an anti-CD40 antibody can be bleselumab. Bleselumab can comprise an amino acid sequence with greater than 70%, greater than 75%, greater than 80%, greater than 85%, greater than 90%, greater than 95% or greater than 99% homology to SEQ ID NO: 50. A light chain of an anti-CD40 antibody can comprise a CDR. A light chain of an anti-CD40 antibody can comprise a CDR with SEQ ID NO: 51. A light chain of an anti-CD40 antibody can comprise a CDR with SEQ ID NO: 52. A light chain of an anti-CD40 antibody can comprise a CDR with SEQ ID NO: 53. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 51. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 52. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 53. The two-dimensional structure of the bleselumab light chain is shown in FIG. 19.

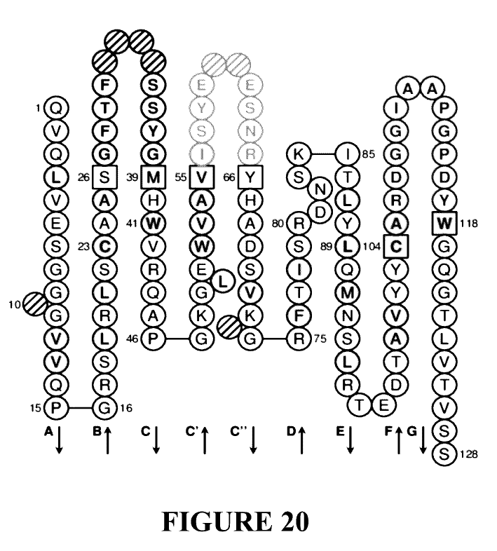

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be lucatumumab. Lucatumumab can comprise an amino acid sequence with greater than 70%, greater than 75%, greater than 80%, greater than 85%, greater than 90%, greater than 95% or greater than 99% homology to SEQ ID NO: 54. A heavy chain of an anti-CD40 antibody can comprise a CDR. A heavy chain of an anti-CD40 antibody can comprise a CDR with SEQ ID NO: 55. A heavy chain of an anti-CD40 antibody can comprise a CDR with SEQ ID NO: 56. A heavy chain of an anti-CD40 antibody can comprise a CDR with SEQ ID NO: 57. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 55. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 56. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 57. The two-dimensional structure of the lucatumumab heavy chain is shown in FIG. 20.

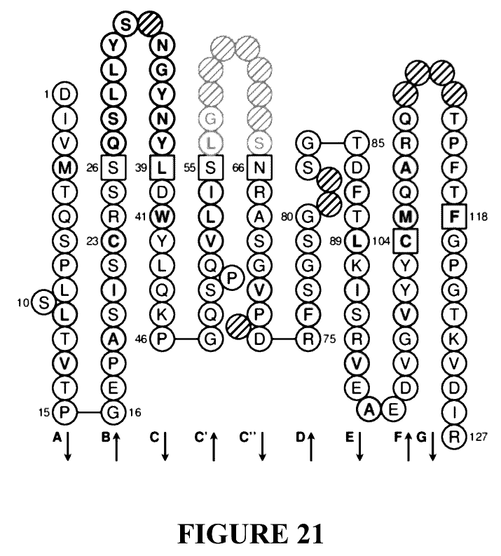

An antibody construct can comprise an antibody light chain. A light chain can be a light chain of an anti-CD40 antibody which can bind a CD40 antigen. A light chain of an anti-CD40 antibody can be Lucatumumab. Lucatumumab can comprise an amino acid sequence with greater than 70%, greater than 75%, greater than 80%, greater than 85%, greater than 90%, greater than 95% or greater than 99% homology to SEQ ID NO: 58. A light chain of an anti-CD40 antibody can comprise a CDR. A light chain of an anti-CD40 antibody can comprise a CDR with SEQ ID NO: 59. A light chain of an anti-CD40 antibody can comprise a CDR with SEQ ID NO: 60. A light chain of an anti-CD40 antibody can comprise a CDR with SEQ ID NO: 61. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 59. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 60. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 61. The two-dimensional structure of the lucatumumab light chain is shown in FIG. 21.

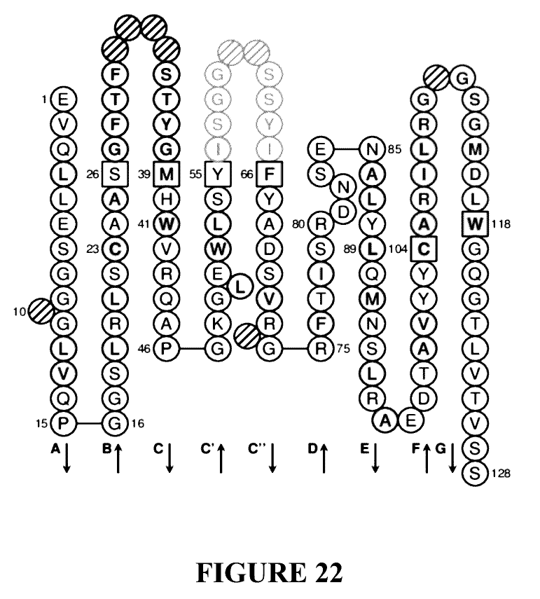

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be ADC-1013. ADC-1013 can comprise an amino acid sequence with greater than 70%, greater than 75%, greater than 80%, greater than 85%, greater than 90%, greater than 95% or greater than 99% homology to SEQ ID NO: 62. A heavy chain of an anti-CD40 antibody can comprise a CDR. A heavy chain of an anti-CD40 antibody can comprise a CDR with SEQ ID NO: 63. A heavy chain of an anti-CD40 antibody can comprise a CDR with SEQ ID NO: 64. A heavy chain of an anti-CD40 antibody can comprise a CDR with SEQ ID NO: 65. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 63. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 64. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 65. The two-dimensional structure of the ADC-1013 heavy chain is shown in FIG. 22.

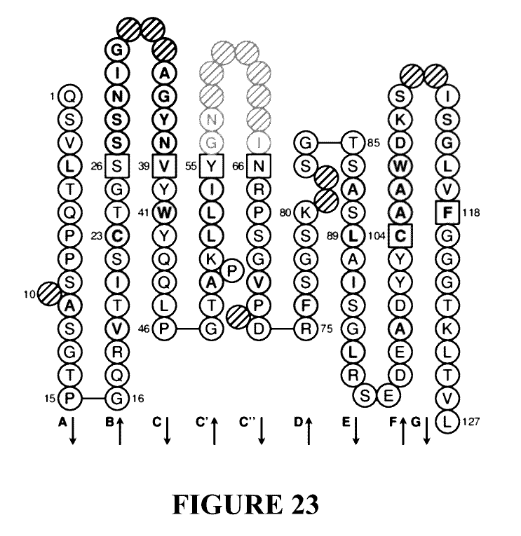

An antibody construct can comprise an antibody light chain. A light chain can be a light chain of an anti-CD40 antibody which can bind a CD40 antigen. A light chain of an anti-CD40 antibody can be ADC-1013. ADC-1013 can comprise an amino acid sequence with greater than 70%, greater than 75%, greater than 80%, greater than 85%, greater than 90%, greater than 95% or greater than 99% homology to SEQ ID NO: 66. A light chain of an anti-CD40 antibody can comprise a CDR. A light chain of an anti-CD40 antibody can comprise a CDR with SEQ ID NO: 67. A light chain of an anti-CD40 antibody can comprise a CDR with SEQ ID NO: 68. A light chain of an anti-CD40 antibody can comprise a CDR with SEQ ID NO: 69. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 67. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 68. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 69. The two-dimensional structure of the ADC-1013 light chain is shown in FIG. 23.

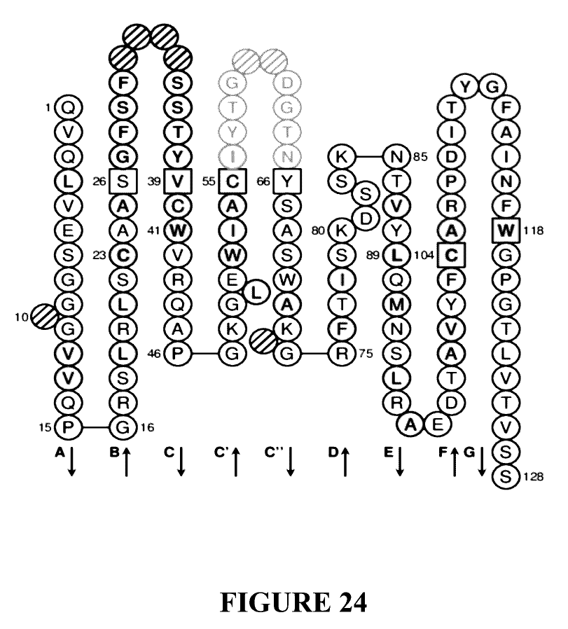

An antibody construct can comprise an antibody heavy chain. A heavy chain can be a heavy chain of an anti-CD40 antibody which can bind a CD40 antigen. A heavy chain of an anti-CD40 antibody can be the humanized rabbit antibody APX005. APX005 can comprise an amino acid sequence with greater than 70%, greater than 75%, greater than 80%, greater than 85%, greater than 90%, greater than 95% or greater than 99% homology to SEQ ID NO: 70. A heavy chain of an anti-CD40 antibody can comprise a CDR. A heavy chain of an anti-CD40 antibody can comprise a CDR with SEQ ID NO: 71. A heavy chain of an anti-CD40 antibody can comprise a CDR with SEQ ID NO: 72. A heavy chain of an anti-CD40 antibody can comprise a CDR with SEQ ID NO: 73. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 71. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 72. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 73. The two-dimensional structure of the APX005 heavy chain is shown in FIG. 24.

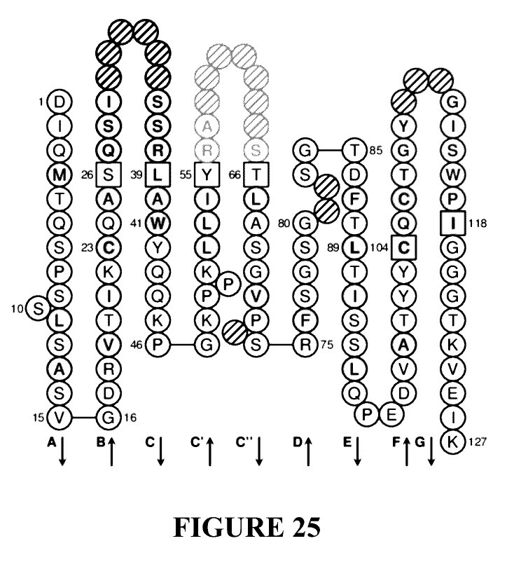

An antibody construct can comprise an antibody light chain. A light chain can be a light chain of an anti-CD40 antibody which can bind a CD40 antigen. A light chain of an anti-CD40 antibody can be the humanized rabbit antibody APX005. APX005 can comprise an amino acid sequence with greater than 70%, greater than 75%, greater than 80%, greater than 85%, greater than 90%, greater than 95% or greater than 99% homology to SEQ ID NO: 74. A light chain of an anti-CD40 antibody can comprise a CDR. A light chain of an anti-CD40 antibody can comprise a CDR with SEQ ID NO: 75. A light chain of an anti-CD40 antibody can comprise a CDR with SEQ ID NO: 76. A light chain of an anti-CD40 antibody can comprise a CDR with SEQ ID NO: 77. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 75. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 76. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 77. The two-dimensional structure of the APX005 light chain is shown in FIG. 25.

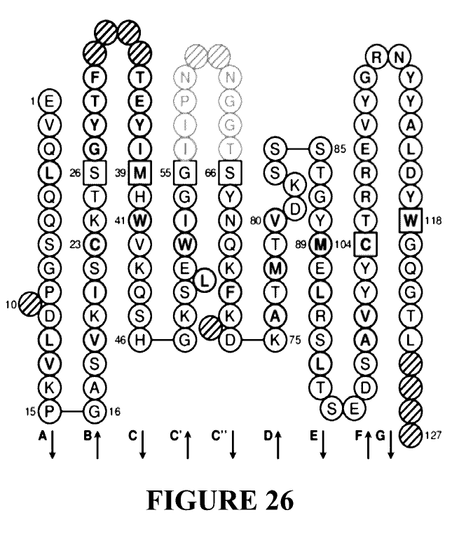

An antibody construct can comprise an antibody heavy chain. A heavy chain can be a heavy chain of an anti-CD40 antibody which can bind a CD40 antigen. A heavy chain of an anti-CD40 antibody can be Chi Lob 7/4. Chi Lob 7/4 can comprise an amino acid sequence with greater than 70%, greater than 75%, greater than 80%, greater than 85%, greater than 90%, greater than 95% or greater than 99% homology to SEQ ID NO: 78. A heavy chain of an anti-CD40 antibody can comprise a CDR. A heavy chain of an anti-CD40 antibody can comprise a CDR with SEQ ID NO: 79. A heavy chain of an anti-CD40 antibody can comprise a CDR with SEQ ID NO: 80. A heavy chain of an anti-CD40 antibody can comprise a CDR with SEQ ID NO: 81. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 79. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 80. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 81. The two-dimensional structure of the Chi Lob 7/4 heavy chain is shown in FIG. 26.

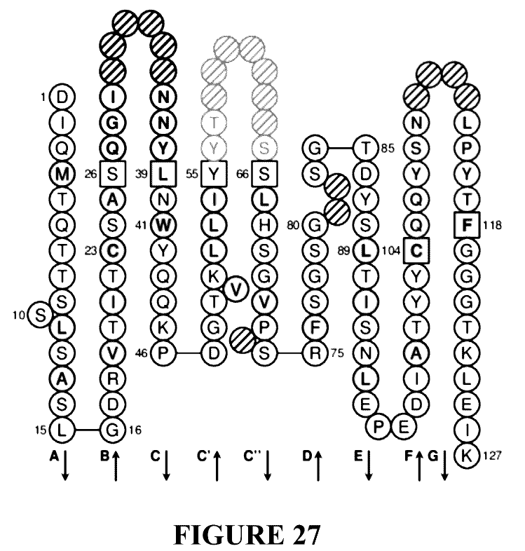

An antibody construct can comprise an antibody light chain. A light chain can be a light chain of an anti-CD40 antibody which can bind a CD40 antigen. A light chain of an anti-CD40 antibody can be Chi Lob 7/4. Chi Lob 7/4 can comprise an amino acid sequence with greater than 70%, greater than 75%, greater than 80%, greater than 85%, greater than 90%, greater than 95% or greater than 99% homology to SEQ ID NO: 82. A light chain of an anti-CD40 antibody can comprise a CDR. A light chain of an anti-CD40 antibody can comprise a CDR with SEQ ID NO: 83. A light chain of an anti-CD40 antibody can comprise a CDR with SEQ ID NO: 84. A light chain of an anti-CD40 antibody can comprise a CDR with SEQ ID NO: 85. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 83. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 84. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 85. The two-dimensional structure of the Chi Lob 7/4 light chain is shown in FIG. 27.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040-G1WT. SBT-040-G1WT be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGTCAGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACAGCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCCCGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 8). SBT-040-G1WT can be expressed from a DNA sequence comprising greater than 70%, greater than 75%, greater than 80%, greater than 85%, greater than 90%, greater than 95% or greater than 99% homology to SEQ ID NO: 8. A variable region of SBT-040-G1WT can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G1WT can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1WT expressed from SEQ ID NO: 8, or expressed from a DNA sequence comprising greater than 70% homology to SEQ ID NO: 8 can have a dissociation constant (K_{d}) for CD40 that is less than 10 nM. Anti-CD40 antibodies comprising SBT-040-G1WT expressed from DNA sequence comprising SEQ ID NO: 8, or comprising greater than 70% homology to SEQ ID NO: 8 can have a dissociation constant (K_{d}) for CD40 that is less than 1 nM, less than 100 pM, less than 10 pM, less than 1 pM, or less than 0.1 pM. SBT-040-G1WT can be expressed with any anti-CD40 light chain or fragment thereof. SBT-040-G1WT can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1WT can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSS VVTVPSSNFGTQTYTCNVDHKPSNTKV DKTVEPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPI EKTISKAKGQPREPQVYTLPPSREEMTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 15). SBT-040-G1WT can comprise an amino acid sequence with greater than 70%, greater than 75%, greater than 80%, greater than 85%, greater than 90%, greater than 95% or greater than 99% homology to SEQ ID NO: 15. SBT-040-G1WT can comprise an amino acid sequence QVQLVQSGAEVKKPGAS VKVSCKASGYTFTGYYMHWVRQAPGQGLEWMGWINPDSGGT NYAQKFQGRVTMTRDTSISTAYMELNRLRS DDTAVYYCARDQPLGYCTNGVCSYFDYWG QGTLVTVSS (SEQ ID NO: 20). A variable region of SBT-040-G1WT can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 10 nM. Anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 1 nM, less than 100 pM, less than 10 pM, less than 1 pM, or less than 0.1 pM. SBT-040-G1WT can be purified. SBT-040-G1WT can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040 VH-hIgG1 wt. SBT-040 VH-hIgG1 wt can comprise an amino acid sequence with greater than 70%, greater than 75%, greater than 80%, greater than 85%, greater than 90%, greater than 95% or greater than 99% homology to SEQ ID NO: 22. A heavy chain of an anti-CD40 antibody can comprise a CDR. A heavy chain of an anti-CD40 antibody can comprise a CDR with SEQ ID NO: 23. A heavy chain of an anti-CD40 antibody can comprise a CDR with SEQ ID NO: 24. A heavy chain of an anti-CD40 antibody can comprise a CDR with SEQ ID NO: 25. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 23. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 24. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 25.

A heavy chain of an anti-CD40 antibody can be an IgG2 isotype. A heavy chain of an anti-CD40 antibody can be SBT-040-G2. SBT-040-G2 be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCGCAAATGTTGTGTCGAGTGCCCACCGTGC CCAGCACCACCTGTGGCAGGACCGTCAGTCTTCCTCTTCCCCCCAAAACCCAAGGACAC CCTCATGATCTCCCGGACCCCTGAGGTCACGTGCGTGGTGGTGGACGTGAGCCACGAAG ACCCCGAGGTCCAGTTCAACTGGTACGTGGACGGCGTGGAGGTGCATAATGCCAAGAC AAAGCCACGGGAGGAGCAGTTCAACAGCACGTTCCGTGTGGTCAGCGTCCTCACCGTTG TGCACCAGGACTGGCTGAACGGCAAGGAGTACAAGTGCAAGGTCTCCAACAAAGGCCT CCCAGCCCCCATCGAGAAAACCATCTCCAAAACCAAAGGGCAGCCCCGAGAACCACAG GTGTACACCCTGCCCCCATCCCGGGAGGAGATGACCAAGAACCAGGTCAGCCTGACCT GCCTGGTCAAAGGCTTCTACCCCAGCGACATCGCCGTGGAGTGGGAGAGCAATGGGCA GCCGGAGAACAACTACAAGACCACACCTCCCATGCTGGACTCCGACGGCTCCTTCTT CCTCTACAGCAAGCTCACCGTGGACAAGAGCAGGTGGCAGCAGGGGAACGTCTT CTCATGCTCCGTGATGCATGAGGCTCTGCACAACCACTACACGCAGAAGAGCCTCTCCC TGTCTCCGGGTAAATGA (SEQ ID NO: 7). SBT-040-G2 can be expressed from a DNA sequence comprising greater than 70%, greater than 75%, greater than 80%, greater than 85%, greater than 90%, greater than 95% or greater than 99% homology to SEQ ID NO: 7. A variable region of SBT-040-G2 can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G2 can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G2 expressed from SEQ ID NO: 7, or expressed from a DNA sequence comprising greater than 70% homology to SEQ ID NO: 7 can have a dissociation constant (K_{d}) for CD40 that is less than 10 nM. Anti-CD40 antibodies comprising SBT-040-G2 expressed from DNA sequence comprising SEQ ID NO: 7, or comprising greater than 70% homology to SEQ ID NO: 7 can have a dissociation constant (K_{d}) for CD40 that is less than 1 nM, less than 100 pM, less than 10 pM, less than 1 pM, or less than 0.1 pM. SBT-040-G2 can be expressed with any anti-CD40 light chain or fragment thereof. SBT-040-G2 can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G2 can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVERKCCVECPPCPAPPVAGPSVFLFPPKPKDTLMISRTPEVTCVVVDVSHEDPEVQFNW YVDGVEVHNAKTKPREEQFNSTFRVVSVLTVVHQDWLNGKEYKCKVSNKGLPAPIEKTISK TKGQPREPQVYTLPPSREEMTKNQVSLTCLVKGFYPSDIAVEWESNGQPENNYKTTPPMLD SDGSFFLYSKLTVDKSRWQQGNVFSCSVMHEALHNHYTQKSLSLSPGK (SEQ ID NO: 14). SBT-040-G2 can comprise an amino acid sequence with greater than 70%, greater than 75%, greater than 80%, greater than 85%, greater than 90%, greater than 95% or greater than 99% homology to SEQ ID NO: 14. SBT-040-G1WT can comprise an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSS (SEQ ID NO: 20). A variable region of SBT-040-G2 can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 10 nM. Anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 1 nM, less than 100 pM, less than 10 pM, less than 1 pM, or less than 0.1 pM. SBT-040-G2 can be purified. SBT-040-G2 can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

An antibody construct can comprise an antibody with modifications occurring at least at one amino acid residue. Modifications can be substitutions, additions, mutations, deletions, or the like. An antibody modification can be an insertion of an unnatural amino acid.

An antibody construct can comprise a light chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An antibody construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A heavy chain can be the heavy chain of an anti-CD40 antibody which can bind to the CD40 antigen.

An antibody construct can be an IgG1 isotype. An antibody construct can be an IgG2 isotype. An antibody construct can be an IgG3 isotype. An antibody construct can be an IgG4 isotype. An antibody construct can be of a hybrid isotype comprising constant regions from two or more isotypes. An antibody construct can be an anti-CD40 antibody, in which the anti-CD40 antibody can be a monoclonal human antibody comprising a wild-type sequence of an IgG1 isoform, in particular, at an Fc region of the antibody.

Antibody constructs disclosed herein can be non-natural, designed, and/or engineered. Antibody constructs disclosed herein can be non-natural, designed, and/or engineered scaffolds comprising an antigen binding domain. Antibody constructs disclosed herein can be non-natural, designed, and/or engineered antibodies. Antibody constructs can be monoclonal antibodies. Antibody constructs can be human antibodies. Antibody constructs can be humanized antibodies. Antibody constructs can be monoclonal humanized antibodies. Antibody constructs can be recombinant antibodies.

Sequences that can be used to produce antibodies for antibody constructs can include leader sequences. Leader sequences can be signal sequences. Leader sequences useful with the compositions and methods described herein can include, but are not limited to, a DNA sequence comprising ATGAGGCTCCCTGCTCAGCTCCTGGGGCTCCTGCTGCTCTGGTTCCCAGGTTCCAGATGC (SEQ ID NO: 2) or ATGGACTGGACCTGGAGGATCCTCTTCTTGGTGGCAGCAGCCACAGGAGCCCACTCC (SEQ ID NO: 12), or an amino acid sequence comprising MRLPAQLLGLLLLWFPGSRC (SEQ ID NO: 5) and MDWTWRILFLVAAATGAHS (SEQ ID NO: 19). A leader sequence can comprise a DNA sequence with greater than 70%, greater than 75%, greater than 80%, greater than 85%, greater than 90%, greater than 95% or greater than 99% homology to SEQ ID NO: 2 or SEQ ID NO: 12. A leader sequence can comprise an amino acid sequence with greater than 70%, greater than 75%, greater than 80%, greater than 85%, greater than 90%, greater than 95% or greater than 99% homology to SEQ ID NO: 5 or SEQ ID NO: 19. Additionally, one skilled in the art would recognize that these same concepts can apply to anti-CD40 antibodies created for use in the veterinary sciences and/or in laboratory animals.

An antigen binding domain of an antibody construct can be selected in order to recognize an antigen. For example, an antigen can be a cell surface marker on target cells associated with a disease or condition. An antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be a peptide presented in a major histocompatibility complex by cell. As another example, a cell surface marker recognized by the antigen binding domain can include macromolecules associated with viral and bacterial diseases or infections, autoimmune diseases and cancerous diseases. An antigen can be CD40 and an antigen binding domain can recognize a CD40 antigen. An antigen can be a tumor antigen or fragment thereof. A tumor antigen can be any antigen listed on tumor antigen databases, such as TANTIGEN, or peptide databases for T cell-defined tumor antigens, such as the Cancer Immunity Peptide database. A tumor antigen can also be any antigen listed in the review by Chen (Chen, Cancer Immun 2004 [updated 2004 Mar 10; cited 2004 Apr 1]). Note that the ‘antibody’ can recognize the ‘tumor antigen’ or a peptide derived thereof, bound to an MHC molecule. An antigen can be CDS, CD19, CD20, CD25, CD37, CD30, CD33, CD45, CAMPATH-1, BCMA, CS-1, PD-L1, B7-H3, B7-DC, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin, PSCA, MAGE Al, sLe(animal), CYP1B1, PLAV1, GM3, BORIS, Tn, GloboH, ETV6-AML, NY-BR-1, RGSS, SART3, STn, Carbonic anhydrase IX, PAXS, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1. An antigen binding domain can be capable of recognizing a single antigen. An antigen binding domain can be capable of recognizing two or more different antigens.

An antibody construct can comprise an Fc region with an Fc domain. An Fc domain is a structure that can bind to Fc receptors. An antibody construct can comprise an Fc domain. Fc domains can be bound by Fc receptors (FcRs). Fc domains can be from antibodies. An Fc domain can be at least 80% homologous to an Fc domain from an antibody. An Fc region can be in a scaffold. An Fc region with an Fc domain can be in an antibody scaffold. An Fc region with an Fc domain can be in a non-antibody scaffold. An antibody construct can comprise an Fc region with an Fc domain in an antibody scaffold. An antibody construct can comprise an Fc region with an Fc domain in a non-antibody scaffold. An Fc domain can be in a scaffold. An Fc domain can be in an antibody scaffold. An Fc domain can be in a non-antibody scaffold. An antibody construct can comprise an Fc domain in an antibody scaffold. An antibody construct can comprise an Fc domain in a non-antibody scaffold. Fc domains of antibodies, including those of the present disclosure, can be bound by Fc receptors (FcRs). Fc domains can be a portion of the Fc region of an antibody. FcRs can bind to an Fc domain of an antibody. FcRs can bind to an Fc domain of an antibody bound to an antigen. FcRs can be organized into classes (e.g., gamma (y), alpha (a) and epsilon (c)) based on the class of antibody that the FcR recognizes. The FcaR class can bind to IgA and includes several isoforms, FcαRI (CD89) and FcαμR. The FcγR class can bind to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant. An FcγRIIIA (CD16a) can be an FcγRIIIA (CD16a) V158 variant. Each FcγR isoform can differ in affinity to the Fc region of the IgG antibody. For example, FcγRI can bind to IgG with greater affinity than FcγRII or FcγRIII. The affinity of a particular FcγR isoform to IgG can be controlled, in part, by a glycan (e.g., oligosacccharaide) at position CH₂ 84.4 of the IgG antibody. For example, fucose containing CH₂ 84.4 glycans can reduce IgG affinity for FcγRIIIA In addition, GO glucans can have increased affinity for FcγRIIIA due to the lack of galactose and terminal GlcNAc moiety.

Binding of an Fc domain to an FcR can enhance an immune response. FcR-mediated signaling that can result from an Fc region binding to an FcR can lead to the maturation of immune cells. FcR-mediated signaling that can result from an Fc domain binding to an FcR can lead to the maturation of dendritic cells. FcR-mediated signaling that can result from an Fc domain binding to an FcR can lead to more efficient immune cell antigen uptake and processing. FcR-mediated signaling that can result from an Fc region binding to an FcR can lead to more efficient dendritic cell antigen uptake and processing. FcR-mediated signaling that can result from an Fc region binding to an FcR can increase antigen presentation. FcR-mediated signaling that can result from an Fc region binding to an FcR can increase antigen presentation by immune cells. FcR-mediated signaling that can result from an Fc region binding to an FcR can increase antigen presentation by antigen presenting cells. FcR-mediated signaling that can result from an Fc domain binding to an FcR can increase antigen presentation by dendritic cells. FcR-mediated signaling that can result from an Fc domain binding to an FcR can promote the expansion and activation of T cells. FcR-mediated signaling that can result from an Fc domain binding to an FcR can promote the expansion and activation of CD8⁺ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a T_{H}1 cell response).

A modification in the amino acid sequence of the antibody construct can alter the recognition of an FcR for the Fc domain. However, such modifications can still allow for FcR-mediated signaling. A modification can be a substitution of an amino acid at a residue (e.g., wildtype) for a different amino acid at that residue. A modification can permit binding of an FcR to a site on the Fc region of an antibody construct that the FcR may not otherwise bind to. A modification can increase binding affinity of an FcR to the Fc domain of an antibody construct that the FcR may have reduced binding affinity for. A modification can decrease binding affinity of an FcR to a site on the Fc domain of an antibody construct that the FcR may have increased binding affinity for. A modification can increase the subsequent FcR-mediated signaling after Fc binding to an FcR.

An antibody construct can comprise an Fc region with at least one amino acid change as compared to the sequence of the wild-type Fc region. An antibody construct can comprise an Fc domain with at least one amino acid change as compared to the sequence of the wild-type Fc domain. An amino acid change in an Fc region of an antibody construct can allow the antibody construct to bind to at least one Fc receptor with greater affinity compared to a wild-type Fc region. An amino acid change in an Fc domain of an antibody construct can allow the antibody to bind to at least one Fc receptor with greater affinity compared to a wild-type Fc domain. An Fc region can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc region can be an Fc region of an anti-CD40 antibody. An Fc domain can be an Fc domain of an anti-CD40 antibody. An Fc region can contain an Fc domain. An Fc region can be an Fc domain.

An antibody construct can be a monoclonal anti-CD40 human antibody comprising a sequence of the IgG1 isoform that has been modified from the wildtype IgG1 sequence. A modification can comprise a substitution at more than one amino acid residue such as at 5 different amino acid residues including L235V/F243L/R292P/Y300L/P396L (SBT-040-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (SBT-040-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (SBT-040-G1AAA). The 3 amino acid residues can be located in a portion of an antibody sequence which can encode an Fc region of the antibody and in particular, can be located in portions of the Fc region that can bind Fc receptors (i.e., the Fc domain).

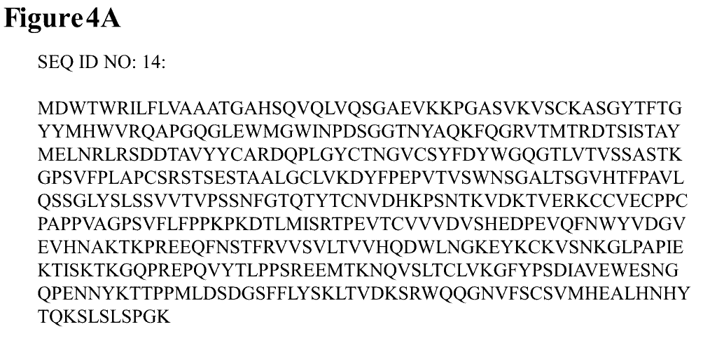

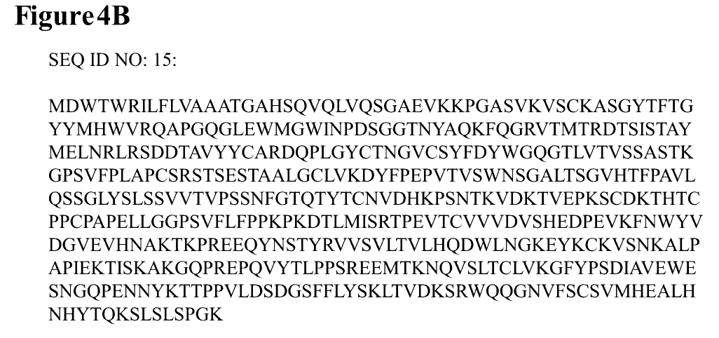

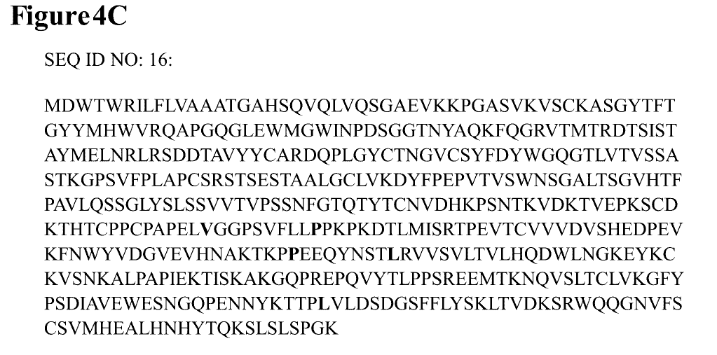

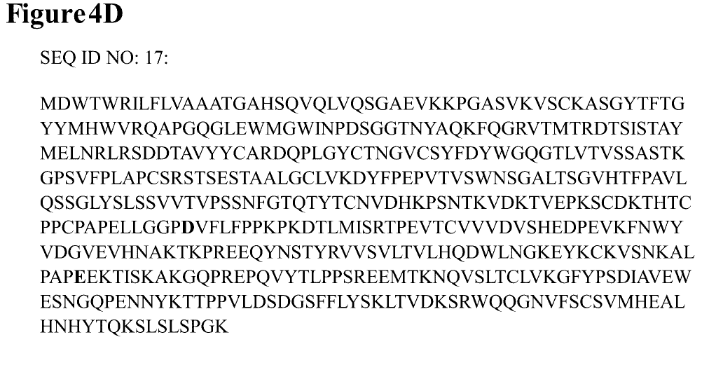

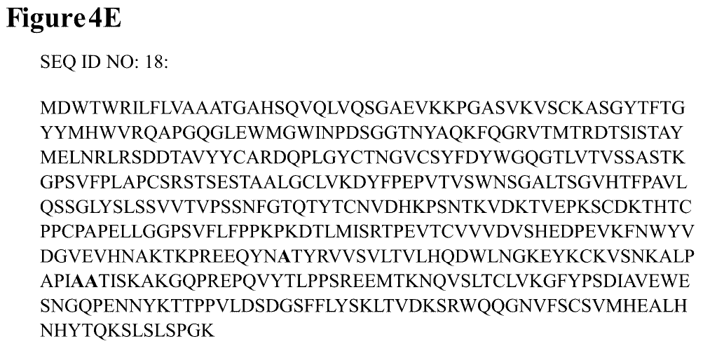

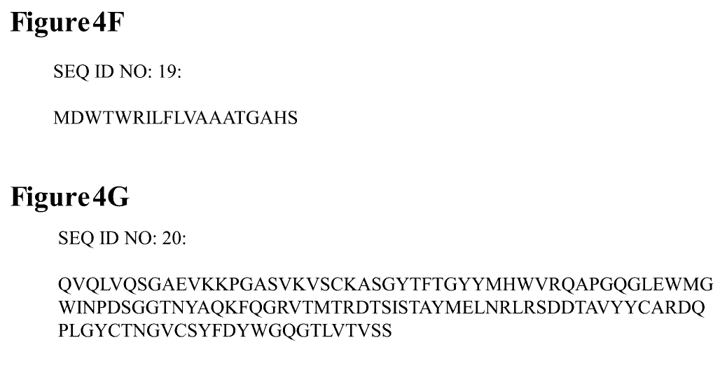

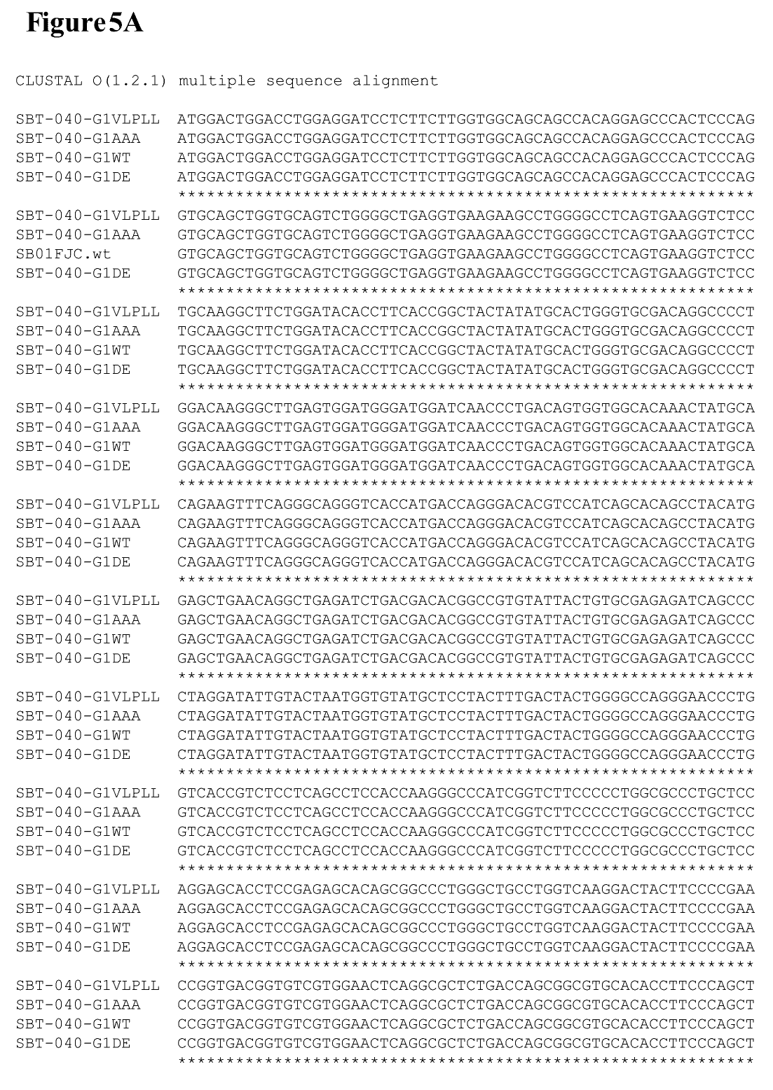

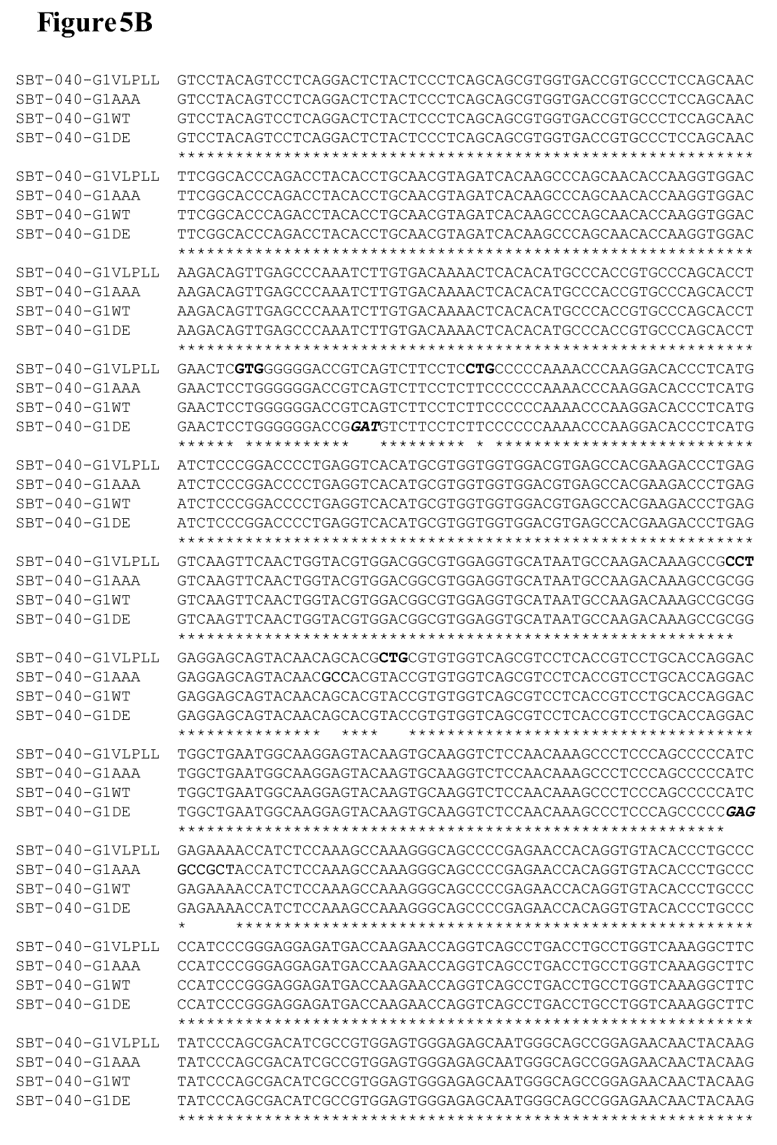

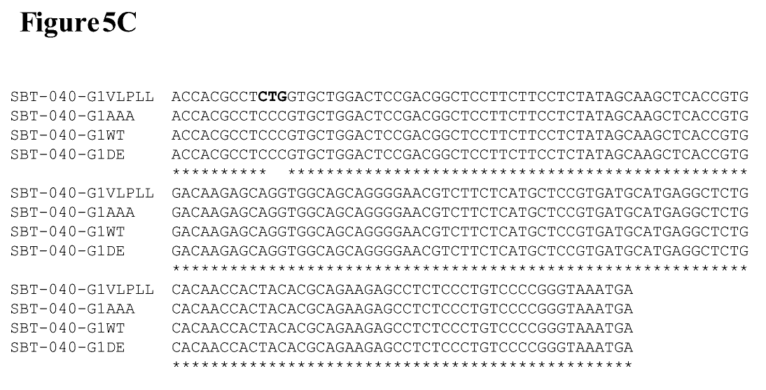

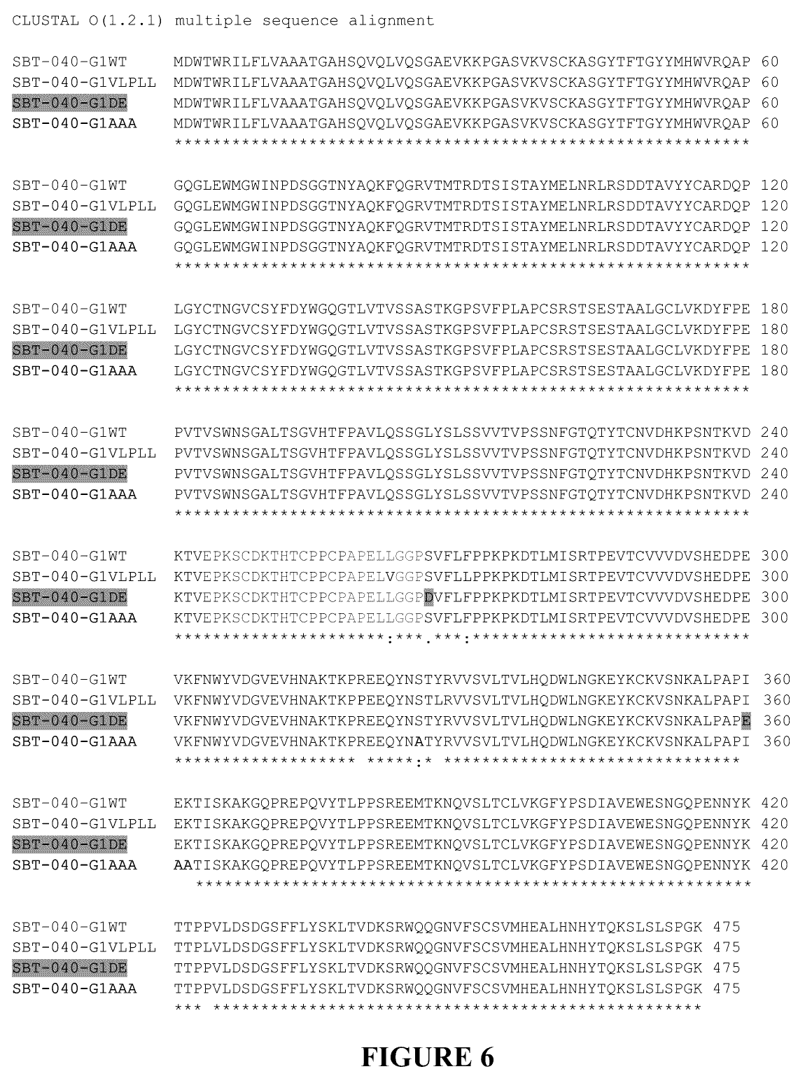

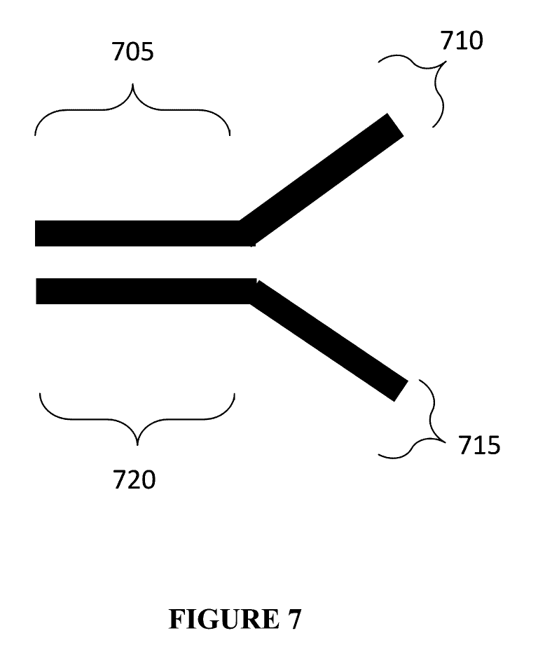

Binding of Fc receptors to an Fc region can be affected by amino acid substitutions. For example, FIG. 4C illustrates SBT-040-VLPLL, which is an antibody with an amino acid sequence (SEQ ID NO: 16) of a heavy chain of human anti-CD40 monoclonal antibody with modifications to a wild-type IgG1 Fc domain (L235V/F243L/R292P/Y300L/P396L). Binding of some Fc receptors to the Fc region of SBT-040-VLPLL can be enhanced compared to wild-type by as result of the L235V/F243L/R292P/Y300L/P396L amino acid modifications. However, binding of other Fc receptors to the Fc region of SBT-040-VLPLL can be reduced compared to wild-type by the L235V/F243L/R292P/Y300L/P396L amino acid modifications. For example, the binding affinities of SBT-040-VLPLLto FcγRIIIA and to FcγRIIA can be enhanced compared to wild-type whereas the binding affinity of SBT-040-VLPLLto FcγRIIB can be reduced compared to wild-type. FIG. 4D illustrates an SBT-040-DE antibody, which is an antibody with an amino acid sequence (SEQ ID NO: 17) of a heavy chain of human anti-CD40 monoclonal antibody with modifications to a wild-type IgG1 Fc domain (S239D/I332E). Binding of Fc receptors to the Fc region of SBT-040-DE can be enhanced compared to wild-type as a result of the S239D/I332E amino acid modification. However, binding of some Fc receptors to the Fc region of SBT-040-G1DE can be reduced compared to wild-type by S239D/I332E amino acid modification. For example, the binding affinities of SBT-040-DE to FcγRIIIA and to FcγRIIB can be enhanced compared to wild-type. Binding of Fc receptors to an Fc region of are affected by amino acid substitutions. FIG. 4E illustrates an SBT-040-G1AAA antibody, which is an antibody with an amino acid sequence (SEQ ID NO: 18) of a heavy chain of a human anti-CD40 monoclonal antibody with modifications to a wild-type IgG1 Fc domain (S298A/E333A/K334A). Binding of Fc receptors to an Fc region of SBT-040-G1AAA can be enhanced compared to wild-type as a result of the S298A/E333A/K334A amino acid modification. However, binding of some Fc receptors to the Fc region of SBT-040-G1AAA can be reduced compared to wild-type by S298A/E333A/K334A amino acid modification. Binding affinities of SBT-040-G1AAA to FcγRIIIA can be enhanced compared to wild-type whereas the binding affinity of SBT-040-G1AAA to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

An antibody construct can be a heavy chain of an anti-CD40 antibody. A heavy chain of an anti-CD40 antibody can be SBT-040-G1VLPLL. SBT-040-G1VLPLL be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCGTGGGGGGACCGTCAGTCTTCCTCCTGCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCCTGAGGAGCAGTACAACAGCACGCTGCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCTGGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 9) wherein the DNA sequence comprises DNA nucleotide modifications that correspond to L235V, F243L, R292P, Y300L and P396L amino acid residue modifications compared to a wild-type DNA sequence. SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 9. A variable region of SBT-040-G1VLPLL can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1VLPLL expressed from SEQ ID NO: 9, or expressed from a DNA sequence comprising greater than 70% homology to SEQ ID NO: 9 can have a dissociation constant (K_{d}) for CD40 that is less than 10 nM. Anti-CD40 antibodies comprising SBT-040-G1VLPLL expressed from DNA sequence comprising SEQ ID NO: 9, or comprising greater than 70% homology to SEQ ID NO: 9 can have a dissociation constant (K_{d}) for CD40 that is less than 1 nM, less than 100 pM, less than 10 pM, less than 1 pM, or less than 0.1 pM. SBT-040-G1VLPLL can be expressed with any anti-CD40 light chain or fragment thereof. SBT-040-G1VLPLL can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1VLPLL can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELVGGPSVFLLPPKPKDTLMISRTPEVTCVVVDVSHEDPEV KFNWYVDGVEVHNAKTKPPEEQYNSTLRVVSVLTVLHQDWLNGKEYKCKVSNKALPAPIE KTISKAKGQPREPQVYTLPPSREEMTKNQVSLTCLVKGFYPSDIAVEWESNGQPENNYKTTP LVLDSDGSFFLYSKLTVDKSRWQQGNVFSCSVMHEALHNHYTQKSLSLSPGK (SEQ ID NO: 16) wherein the amino acid sequence comprises L235V, F243L, R292P, Y300L, and P396L amino acid residue modifications compared to a wild-type amino acid sequence. SBT-040-G1VLPLL can comprise an amino acid sequence with greater than 70%, greater than 75%, greater than 80%, greater than 85%, greater than 90%, greater than 95% or greater than 99% homology to SEQ ID NO: 16. SBT-040-G1VLPLL can comprise an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSS (SEQ ID NO: 20). A variable region of SBT-040-G1VLPLL can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 10 nM. Anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 1 nM, less than 100 pM, less than 10 pM, less than 1 pM, or less than 0.1 pM. SBT-040-G1VLPLL can be purified. SBT-040-G1VLPLL can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

A heavy chain of an anti-CD40 antibody can be SBT-040-G1DE. SBT-040-G1DE be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGGATGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACAGCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCGAGGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCCCGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 10) wherein the DNA sequence comprises DNA nucleotide modifications that correspond to S239D and I332E amino acid residue modifications compared to a wild-type DNA sequence. SBT-040-G1DE can be expressed from a DNA sequence comprising greater than 70%, greater than 75%, greater than 80%, greater than 85%, greater than 90%, greater than 95% or greater than 99% homology to SEQ ID NO: 10. A variable region of SBT-040-G1DE can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G1DE can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1DE expressed from SEQ ID NO: 10, or expressed from a DNA sequence comprising greater than 70% homology to SEQ ID NO: 10 can have a dissociation constant (K_{d}) for CD40 that is less than 10 nM. Anti-CD40 antibodies comprising SBT-040-G1DE expressed from DNA sequence comprising SEQ ID NO: 10, or comprising greater than 70% homology to SEQ ID NO: 10 can have a dissociation constant (K_{d}) for CD40 that is less than 1 nM, less than 100 pM, less than 10 pM, less than 1 pM, or less than 0.1 pM. SBT-040-G1DE can be expressed with any anti-CD40 light chain or fragment thereof. SBT-040-G1DE can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1DE can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELLGGPDVFLFPPKPKDTLMISRTPEVTCVVVDVSHEDPEV KFNWYVDGVEVHNAKTKPREEQYNSTYRVVSVLTVLHQDWLNGKEYKCKVSNKALPAPE EKTISKAKGQPREPQVYTLPPSREEMTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 17) wherein the amino acid sequence comprises S239D and I332E amino acid residue modifications compared to a wild-type amino acid sequence. SBT-040-G1DE can comprise an amino acid sequence with greater than 70%, greater than 75%, greater than 80%, greater than 85%, greater than 90%, greater than 95% or greater than 99% homology to SEQ ID NO: 17. SBT-040-G1DE can comprise an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSS (SEQ ID NO: 20). A variable region of SBT-040-G1DE can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 10 nM. Anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 1 nM, less than 100 pM, less than 10 pM, less than 1 pM, or less than 0.1 pM. SBT-040-G1DE can be purified. SBT-040-G1DE can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

A heavy chain of an anti-CD40 antibody can be SBT-040-G1AAA. SBT-040-G1AAA be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGTCAGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACGCCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGCCGCTACCATCTCCAAAGCCAAAGGGCAGCCCC GAGAACCACAGGTGTACACCCTGCCCCCATCCCGGGAGGAGATGACCAAGAACCAGGT CAGCCTGACCTGCCTGGTCAAAGGCTTCTATCCCAGCGACATCGCCGTGGAGTGGGAGA GCAATGGGCAGCCGGAGAACAACTACAAGACCACGCCTCCCGTGCTGGACTCCGACGG CTCCTTCTTCCTCTATAGCAAGCTCACCGTGGACAAGAGCAGGTGGCAGCAGGGGAACG TCTTCTCATGCTCCGTGATGCATGAGGCTCTGCACAACCACTACACGCAGAAGAGCCTC TCCCTGTCCCCGGGTAAATGA (SEQ ID NO: 11) wherein the DNA sequence comprises DNA nucleotide modifications that correspond to S298A, E333A, and K334A amino acid residue modifications compared to a wild-type DNA sequence. SBT-040-G1AAA can be expressed from a DNA sequence comprising greater than 70%, greater than 75%, greater than 80%, greater than 85%, greater than 90%, greater than 95% or greater than 99% homology to SEQ ID NO: 11. A variable region of SBT-040-G1AAA can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G1AAA can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1AAA expressed from SEQ ID NO: 11, or expressed from a DNA sequence comprising greater than 70% homology to SEQ ID NO: 11 can have a dissociation constant (K_{d}) for CD40 that is less than 10 nM. Anti-CD40 antibodies comprising SBT-040-G1AAA expressed from DNA sequence comprising SEQ ID NO: 11, or comprising greater than 70% homology to SEQ ID NO: 11 can have a dissociation constant (K_{d}) for CD40 that is less than 1 nM, less than 100 pM, less than 10 pM, less than 1 pM, or less than 0.1 pM. SBT-040-G1AAA can be expressed with any anti-CD40 light chain or fragment thereof. SBT-040-G1AAA can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals. SBT-040-G1AAA can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELLGGPSVFLFPPKPKDTLMISRTPEVTCVVVDVSHEDPEV KFNWYVDGVEVHNAKTKPREEQYNATYRVVSVLTVLHQDWLNGKEYKCKVSNKALPAPI AATISKAKGQPREPQVYTLPPSREEMTKNQVSLTCLVKGFYPSDIAVEWESNGQPENNYKT TPPVLDSDGSFFLYSKLTVDKSRWQQGNVFSCSVMHEALHNHYTQKSLSLSPGK (SEQ ID NO: 18) wherein the amino acid sequence comprises S298A, E333A, and K334A amino acid residue modifications compared to a wild-type amino acid sequence. SBT-040-G1AAA can comprise an amino acid sequence with greater than 70%, greater than 75%, greater than 80%, greater than 85%, greater than 90%, greater than 95% or greater than 99% homology to SEQ ID NO: 18. SBT-040-G1AAA can comprise an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSS (SEQ ID NO: 20). A variable region of SBT-040-G1AAA can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1AAA with SEQ ID NO: 18 or with an amino acid sequence with greater than 70% homology to SEQ ID NO: 18 can have a dissociation constant (K_{d}) for CD40 that is less than 10 nM. Anti-CD40 antibodies comprising SBT-040-G1AAA with SEQ ID NO: 18 or with an amino acid sequence with greater than 70% homology to SEQ ID NO: 18 can have a dissociation constant (Id) for CD40 that is less than 1 nM, less than 100 pM, less than 10 pM, less than 1 pM, or less than 0.1 pM. SBT-040-G1AAA can be purified. SBT-040-G1AAA can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to anti-CD40 antibodies created for use in the veterinary sciences and/or in laboratory animals.

An antibody construct can comprise an antibody heavy chain. A heavy chain can be a heavy chain of a HER2 monoclonal antibody which can bind a HER2 antigen. A heavy chain of an anti-HER2 antibody can be an IgG1 isotype. A heavy chain of an anti-HER2 antibody can be SBT-050 VH-hIgG1 wt (pertuzumab). SBT-050 VH-hIgG1 wt can comprise an amino acid sequence with greater than 70%, greater than 75%, greater than 80%, greater than 85%, greater than 90%, greater than 95% or greater than 99% homology to SEQ ID NO: 30.

A heavy chain of an anti-HER2 antibody can comprise a CDR. A heavy chain of an anti-HER2 antibody can comprise a CDR with SEQ ID NO: 31. A heavy chain of an anti-HER2 antibody can comprise a CDR with SEQ ID NO: 32. A heavy chain of an anti-HER2 antibody antibody can comprise a CDR with SEQ ID NO: 33. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 31. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 32. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 33.

An antibody construct can comprise an antibody light chain. A light chain can be a light chain of a HER2 monoclonal antibody which can bind a HER2 antigen. A light chain of an anti-HER2 antibody can be SBT-050 VL-Ck (pertuzumab). SBT-050 VL-Ck can comprise an amino acid sequence with greater than 70%, greater than 75%, greater than 80%, greater than 85%, greater than 90%, greater than 95% or greater than 99% homology to SEQ ID NO: 34.

A light chain of an anti-HER2 antibody can comprise a CDR. A light chain of an anti-HER2 antibody can comprise a CDR with SEQ ID NO: 35. A light chain of an anti-HER2 antibody can comprise a CDR with SEQ ID NO: 36. A light chain of an anti-HER2 antibody antibody can comprise a CDR with SEQ ID NO: 37.

A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 35. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 36. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 37.

While an antibody construct of the present disclosure can comprise an anti-CD40 antibody with wild-type or modified amino acid sequences encoding the Fc region or Fc domain, the modifications of the Fc region or the Fc domain from the wild-type sequence may not significantly alter binding and/or affinity of the anti-CD40 antibody for CD40. For example, binding and/or affinity of SBT-040-G1WT, SBT-040-G1VLPLL, SBT-040-G1DE, and SBT-040-G1AAA may not be significantly altered by modification of an Fc region or Fc domain amino acid sequence compared to a wild-type sequence. Modifications of an Fc region or Fc domain from a wild-type sequence may not alter binding and/or affinity of antibodies that bind to CD40 in an antibody construct. Additionally, the binding and/or affinity of the antibodies described herein that bind to CD40 and are antibody constructs, for example SBT-040-G1WT, SBT-040-G1VLPLL, SBT-040-G1DE, and SBT-040-G1AAA, may be comparable to the binding and/or affinity of wild-type antibodies that can bind to CD40.

Sequences that can be used to produce antibodies for antibody constructs can include leader sequences. Leader sequences can be signal sequences. Leader sequences useful with the compositions and methods described herein can include, but are not limited to, a DNA sequence comprising ATGAGGCTCCCTGCTCAGCTCCTGGGGCTCCTGCTGCTCTGGTTCCCAGGTTCCAGATGC (SEQ ID NO: 2) or ATGGACTGGACCTGGAGGATCCTCTTCTTGGTGGCAGCAGCCACAGGAGCCCACTCC (SEQ ID NO: 12), or an amino acid sequence comprising MRLPAQLLGLLLLWFPGSRC (SEQ ID NO: 5) and MDWTWRILFLVAAATGAHS (SEQ ID NO: 19). Leader sequence can comprise a DNA sequence with greater than 70%, greater than 75%, greater than 80%, greater than 85%, greater than 90%, greater than 95% or greater than 99% homology to SEQ ID NO: 2 or SEQ ID NO: 12. Leader sequence can comprise an amino acid sequence with greater than 70%, greater than 75%, greater than 80%, greater than 85%, greater than 90%, greater than 95% or greater than 99% homology to SEQ ID NO: 5 or SEQ ID NO: 19. Additionally, one skilled in the art would recognize that these same concepts can apply to antibody constructs comprising anti-CD40 antibodies created for use in the veterinary sciences and/or in laboratory animals.

### Targeting Binding Domain

An antibody construct can further comprise a targeting binding domain. As another example, a targeting binding domain can be linked to an antibody construct. A targeting binding domain of an antibody construct can be selected in order to recognize an antigen. For example, an antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be CD40 and a targeting binding domain can recognize a CD40 antigen. A targeting binding domain can be a CD40 agonist. A targeting domain can be CD40.

### Immune-Stimulatory Compounds

Pattern recognition receptors (PRRs) can recognize pathogen-associated molecular patterns (PAMPs) and damage-associated molecular patterns (DAMPs). A PRR can be membrane bound. A PRR can be cytosolic. A PRR can be a toll-like receptor (TLR). A PRR can be RIG-I-like receptor. A PRR can be a receptor kinase. A PRR can be a C-type lectin receptor. A PRR can be a NOD-like receptor. A PRR can be TLR1, TLR2, TLR3, TLR4, TLRS, TLR6, TLR7, TLR8, TLR9, TLR10, TLR11, TLR12 or TLR13.

A PRR agonist can be pathogen-associated molecular pattern (PAMP) molecule. A PAMP molecule can be a toll-like receptor agonist. A PRR agonist can be a toll-like receptor agonist. A toll-like receptor agonist can be any molecule that acts as an agonist to at least one toll-like receptor. A toll-like receptor agonist can be bacterial lipoprotein. A toll-like receptor agonist can be bacterial peptidoglycans. A toll-like receptor agonist can be double stranded RNA. A toll-like receptor agonist can be lipopolysaccharides. A toll-like receptor agonist can be bacterial flagella. A toll-like receptor agonist can be single stranded RNA. A toll-like receptor can be CpG DNA. A toll-like receptor agonist can be imiquimod. A toll-like receptor agonist can be CL307. A toll-like receptor agonist can be S-27609. A toll-like receptor agonist can be resiquimod. A toll-like receptor agonist can be UC-IV150. A toll-like receptor agonist can be gardiquimod. A toll-like receptor agonist can be motolimod. A toll-like receptor agonist can be VTX-1463. A toll-like receptor agonist can be GS-9620. A toll-like receptor agonist can be GSK2245035. A toll-like receptor agonist can be TMX-101. A toll-like receptor agonist can be TMX-201. A toll-like receptor agonist can be TMX-202. A toll-like receptor agonist can be isatoribine. A toll-like receptor agonist can be AZD8848. A toll-like receptor agonist can be MEDI9197. A toll-like receptor agonist can be 3M-051. A toll-like receptor agonist can be 3M-852. A toll-like receptor agonist can be 3M-052. A toll-like receptor agonist can be 3M-854A. A toll-like receptor agonist can be S-34240. A toll-like receptor agonist can be CL663. A RIG-I agonist can be KIN1148. A RIG-I agonist can be SB-9200. A RIG-I agonist can be KIN700, KIN600, KIN500, KIN100, KIN101, KIN400, or KIN2000. A toll-like receptor agonist can be KU34B.

A PRR agonist can be a damage-associated molecular pattern (DAMP) molecule. A DAMP molecule can be an intracellular protein. A DAMP molecule can be a heat-shock protein. A DAMP molecule can be an HMGB1 protein. A DAMP molecule can be a protein derived from the extracellular matrix that is generated after tissue injury. A DAMP molecule can be a hyaluronan fragment. A DAMP molecule can be DNA. A DAMP molecule can be RNA. A DAMP molecule can be an S100 molecule. A DAMP molecule can be nucleotides. A DAMP molecule can be an ATP. A DAMP molecule can be nucleosides. A DAMP molecule can be an adenosine. A DAMP molecule can be uric acid.

Additionally, stimulator of interferon genes (STING) can act as a cytosolic DNA sensor wherein cytosolic DNA and unique bacterial nucleic acids called cyclic dinucleotides are recognized by STING, and therefore STING agonists. Interferon Regulatory Factor (IRF) agonist can be KIN-100. Non-limiting examples of STING agonists include:

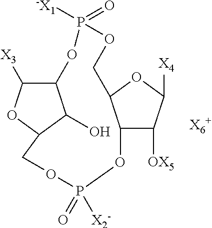

wherein in some embodiments, X₁═X₂═O; X₃=G; X₄=G; X₅═CO(CH₂)₁₂CH₃; X₆=2 TEAH; in some embodiments, X₁═X₂═S [Rₚ,Rₚ]; X₃=G; X₄=A; X₅═H; X₆=2 TEAH; in some embodiments, X₁═X₂═S [Rₚ,Rₚ]; X₃=A; X₄=A; X₅═H; X₆=2 Na; in some embodiments, X₁═X₂═S [Rₚ,Rₚ]; X₃=A; X₄=A; X₅═H; X₆=2 NH₄; and in some embodiments, X₁═X₂═O; X₃=G; X₄=A; X₅═H; X₆=2 TEAH,

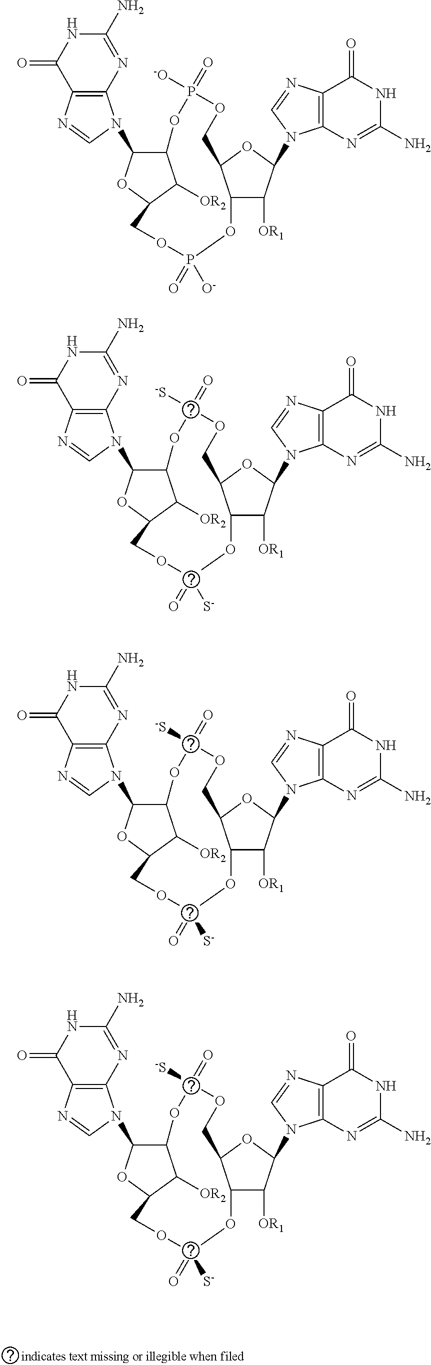

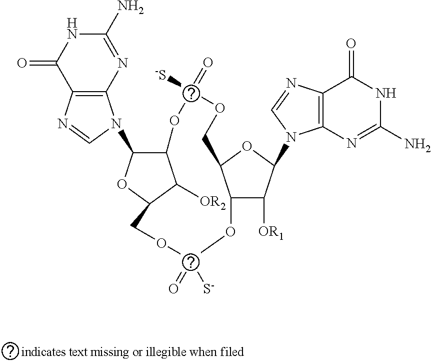

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

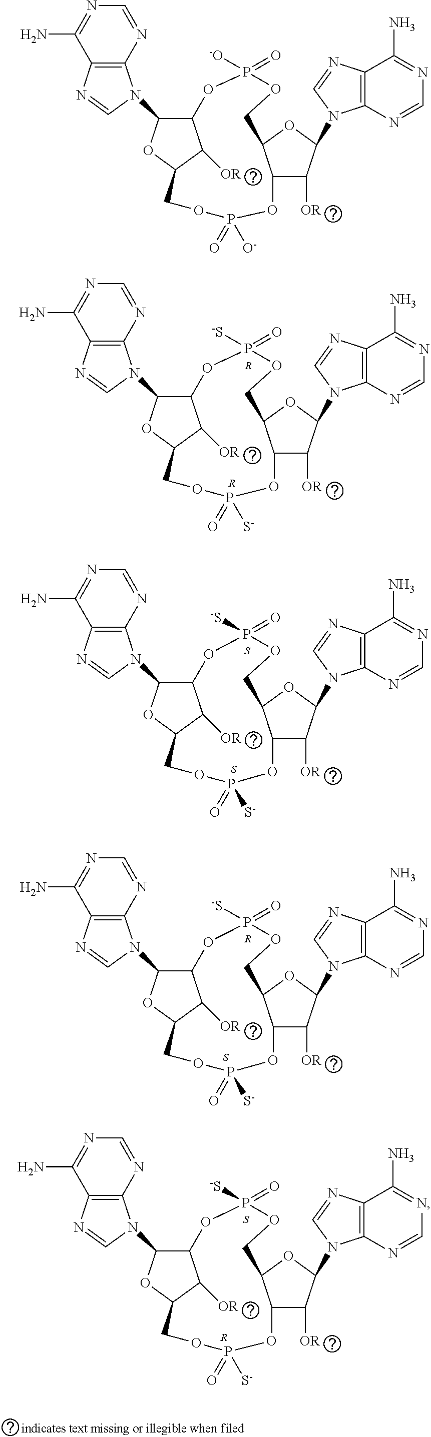

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

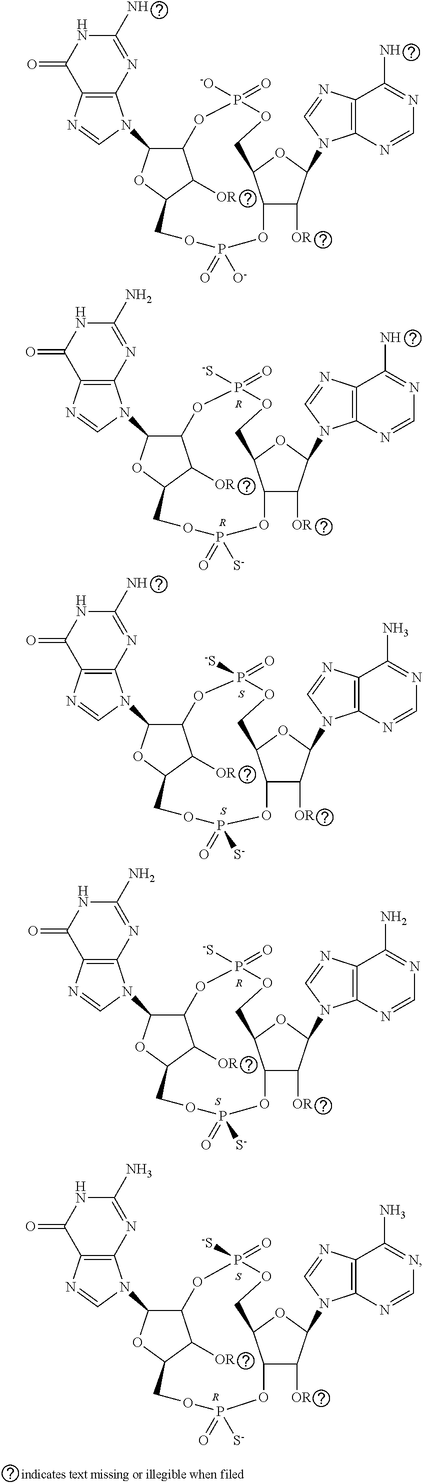

wherein R₁═R₂═H; R₁=propargyl, R₂═H; R₁═H, R₂=propargyl; R₁=allyl, R₂═H; R₁═H, R₂=allyl; R₁=methyl, R₂═H; R₁═H, R₂=methyl; R₁=ethyl, R₂═H; R₁═H, R₂=ethyl; R₁=propyl, R₂═H; R₁═H, R₂=propyl; R₁=benzyl, R₂═H; R₁═H, R₂=benzyl; R₁=myristoyl, R₂═H; R₁═H, R₂=myristoyl; R₁═R₂=heptanoyl; R₁═R₂=hexanoyl; or R₁═R₂=pentanoyl,

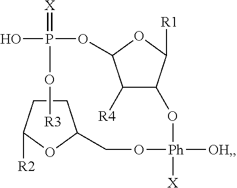

wherein each X is independently O or S, and R3 and R4 are each independently H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ where R′ is H or lower alkyl, —CH₂OH, and —CONH₂, wherein R3 and R4 are not both H,

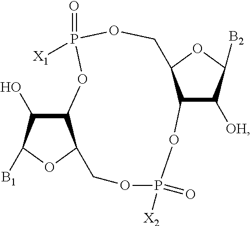

wherein X₁═X₂═O; X₁═X₂═S; or X₁═O and X₂═S,

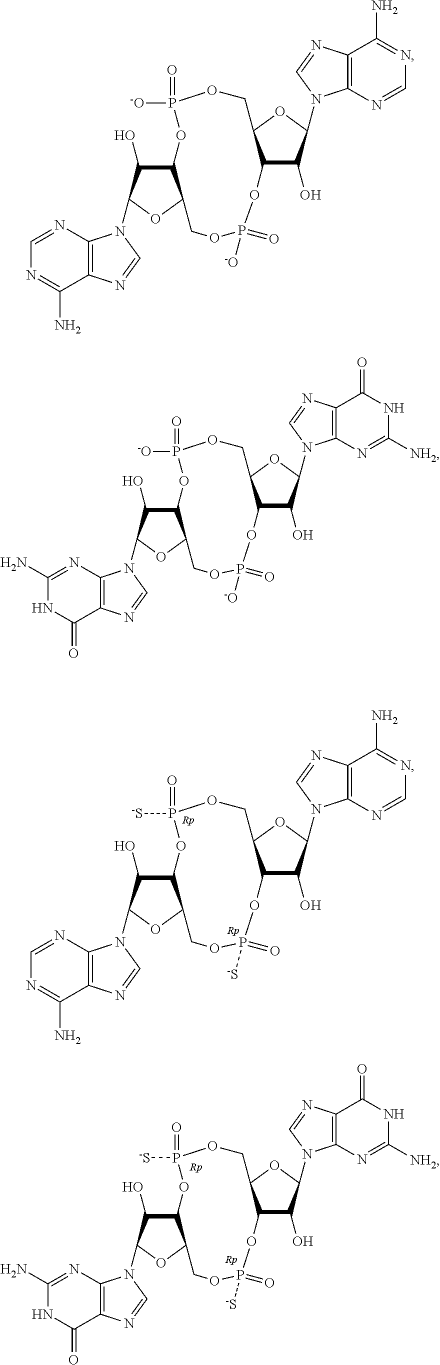

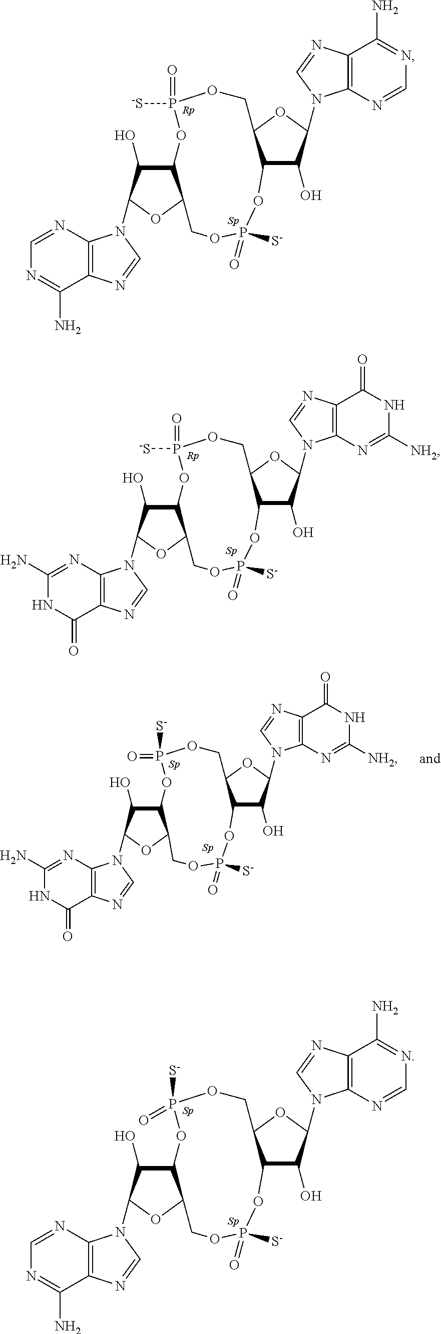

An immune-stimulatory compound can be a PRR agonist. An immune-stimulatory compound can be a PAMP. An immune-stimulatory compound can be a DAMP. An immune-stimulatory compound can be a TLR agonist. An immune-stimulatory compound can be a STING agonist. An immune-stimulatory compound can be a cyclic dinucleotide.

The specificity of the antigen-binding domain to an antigen of an antibody construct disclosed herein can be influenced by the presence of an immune-stimulatory compound. The antigen-binding domain of the antibody construct can bind to an antigen with at least about 10%, about 20%, about 30%, about 40%, about 50%, about 60%, about 70%, about 80%, about 85%, about 90%, about 95%, or about 100% of a specificity of the antigen-binding domain to the antigen in the absence of the immune-stimulatory compound.

The specificity of the Fc domain to an Fc receptor of an antibody construct disclosed herein can be influenced by the presence of an immune-stimulatory compound. The Fc domain of the antibody construct can bind to an Fc receptor with at least about 10%, about 20%, about 30%, about 40%, about 50%, about 60%, about 70%, about 80%, about 85%, about 90%, about 95%, or about 100% of a specificity of the Fc domain to the Fc receptor in the absence of the immune-stimulatory compound.

The affinity of the antigen-binding domain to an antigen of an antibody construct disclosed herein can be influenced by the presence of an immune-stimulatory compound. The antigen-binding domain of the antibody construct can bind to an antigen with at least about 1%, about 5%, about 10%, about 20%, about 30%, about 40%, about 50%, about 60%, about 70%, about 80%, about 85%, about 90%, about 95%, or about 100% of an affinity of the antigen-binding domain to the antigen in the absence of the immune-stimulatory compound.

The affinity of the Fc domain to an Fc receptor of an antibody construct disclosed herein can be influenced by the presence of an immune-stimulatory compound. The Fc domain of the antibody construct can bind to an Fc receptor with at least about 1%, about 5%, about 10%, about 20%, about 30%, about 40%, about 50%, about 60%, about 70%, about 80%, about 85%, about 90%, about 95%, or about 100% of an affinity of the Fc domain to the Fc receptor in the absence of the immune-stimulatory compound.

The K_{d} for binding of an antigen-binding domain to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen in the absence of the immune-stimulatory compound.

The K_{d} for binding of an Fc domain to a Fc receptor in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulator compound.

Affinity can be the strength of the sum total of noncovalent interactions between a single binding site of a molecule, for example, an antibody, and the binding partner of the molecule, for example, an antigen. The affinity can also measure the strength of an interaction between an Fc portion of an antibody and the Fc receptor. Unless indicated otherwise, as used herein, “binding affinity” can refer to intrinsic binding affinity which reflects a 1:1 interaction between members of a binding pair (e.g., antibody and antigen or Fc domain and Fc receptor). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

In some embodiments, an antibody provided herein can have a dissociation constant (K_{d}) of about 1 μM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸ M or less, e.g., from 10⁻⁸ M to 10⁻¹³ M, e.g., from 10⁻⁹ M to 10⁻¹³ M). An affinity matured antibody can be an antibody with one or more alterations in one or more complementarity determining regions (CDRs), compared to a parent antibody, which may not possess such alterations, such alterations resulting in an improvement in the affinity of the antibody for antigen. These antibodies can bind to their antigen with a K_{d} of about 5×10⁻⁹ M, about 2×10⁻⁹ M, about 1×10⁻⁹ M, about 5×10⁻¹ M, about 2×10⁻⁹ M, about 1×10¹⁰ M, about 5×10⁻¹¹ M, about 1×10⁻¹¹ M, about 5×10⁻¹² M, about 1×10⁻¹² M, or less. In some embodiments, the antibody construct can have an increased affinity of at least 1.5-fold, 2-fold, 2.5-fold, 3-fold, 4-fold, 5-fold, 10-fold, 20-fold, or greater as compared to an antibody construct without alterations in one or more complementarity determining regions.

K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000).

Agonism can be described as the binding of a chemical to a receptor to induce a biological response. A chemical can be, for example, a small molecule, a compound, or a protein. An agonist causes a response, an antagonist can block the action of an agonist, and an inverse agonist can cause a response that is opposite to that of the agonist. A receptor can be activated by either endogenous or exogenous agonists.

The molar ratio of an antibody construct immune-stimulatory compound conjugate can refer to the average number of immune-stimulatory compounds conjugated to the antibody construct in a preparation of an antibody construct immune-stimulatory compound conjugate. The molar ratio can be determined, for example, by Liquid Chromatography/Mass Spectrometry (LC/MS), in which the number of immune-stimulatory compounds conjugated to the antibody construct can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of immune-stimulatory compound to antibody can be less than 8. In other embodiments, the molar ratio of immune-stimulatory compound to antibody can be 8, 7, 6, 5, 4, 3, 2, or 1.

### Linkers

The compositions and methods described herein can comprise a linker, e.g., a peptide linker. Linkers of the compositions and methods described herein may not affect the binding of active portions of a conjugate (e.g., active portions include antigen binding domains, Fc domains, targeting binding domains, antibodies, agonists or the like) to a target, which can be a cognate binding partner such as an antigen. A linker sequence can form a linkage between different parts of a composition. A composition can be a conjugate. A conjugate can comprise multiple linkers. These linkers can be the same linkers or different linkers.

Attachment via a linker can involve incorporation of a linker between parts of a composition or conjugate. A linker can be cleavable, non-cleavable, hydrophilic, or hydrophobic. A cleavable linker can be sensitive to enzymes. A cleavable linker can be cleaved by enzymes such as proteases. A cleavable linker can be a valine-citrulline linker or a valine-alanine linker. A valine-citrulline or valine-alanine linker can contain a pentafluorophenyl group. A valine-citrulline or valine-alanine linker can contain a succimide group. A valine-citrulline or valine-alanine linker can contain a para aminobenzoic acid (PABA) group. A valine-citrulline or valine-alanine linker can contain a PABA group and a pentafluorophenyl group. A valine-citrulline or valine-alanine linker can contain a PABA group and a succinimide group. A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly)-(NH₂) linker. A linker can be a THIOMAB linker. A THIOMAB linker can be a (maleimidocaproyl)-(valine-citrulline)-(para-aminobenzyloxycarbonly)-(NH₂) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 21) to an N-terminal GGG motif to regenerate a native amide bond. The linker created can therefore link a moiety attached to the LXPTG recognition motif (SEQ ID NO: 21) with a moiety attached to the N-terminal GGG motif. A linker can be a link created between an unnatural amino acid on one moiety reacting with oxime bond that was formed by modifying a ketone group with an alkoxyamine on another moiety. A moiety can be an antibody construct. A moiety can be an antibody. A moiety can be an immune-stimulatory compound. A moiety can be a targeting binding domain. A linker can be a portion of a linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

### Conjugates

A composition as described herein can be a conjugate. A conjugate can comprise an antibody construct, an immune-stimulatory compound, and a linker. A conjugate can comprise an antibody construct, a pattern recognition receptor (PRR) agonist, and a linker. A conjugate can comprise an antibody construct, a pattern-associated molecular pattern (PAMP) molecule, and a linker. A conjugate can comprise an antibody construct, a damage-associated molecular pattern (DAMP) molecule, and a linker. A conjugate can comprise an antibody construct, a STING agonist, and a linker. A conjugate can comprise an antibody construct, a toll-like receptor agonist molecule, and a linker. A conjugate can comprise an antibody construct, imiquimod, and a linker. A conjugate can comprise an antibody construct, S-27609, and a linker. A conjugate can comprise an antibody construct, CL307, and a linker. A conjugate can comprise an antibody construct, resiquimod, and a linker. A conjugate can comprise an antibody construct, gardiquimod, and a linker. A conjugate can comprise an antibody construct, UC-IV150, and a linker. A conjugate can comprise an antibody construct, KU34B, and a linker. A conjugate can comprise an antibody construct, motolimod, and a linker. A conjugate can comprise an antibody construct, VTX-1463, and a linker. A conjugate can comprise an antibody construct, GS-9620, and a linker. A conjugate can comprise an antibody construct, GSK2245035, and a linker. A conjugate can comprise an antibody construct, TMX-101, and a linker. A conjugate can comprise an antibody construct, TMX-201, and a linker. A conjugate can comprise an antibody construct, TMX-202, and a linker. A conjugate can comprise an antibody construct, isatoribine, and a linker. A conjugate can comprise an antibody construct, AZD8848, and a linker. A conjugate can comprise an antibody construct, MEDI9197, and a linker. A conjugate can comprise an antibody construct, 3M-051, and a linker. A conjugate can comprise an antibody construct, 3M-852, and a linker. A conjugate can comprise an antibody construct, 3M-052, and a linker. A conjugate can comprise an antibody construct, 3M-854A, and a linker. A conjugate can comprise an antibody construct, S-34240, and a linker. A conjugate can comprise an antibody construct, CL663, and a linker. A conjugate can comprise an antibody construct, KIN1148, and a linker. A conjugate can comprise an antibody construct, SB-9200, and a linker. A conjugate can comprise an antibody construct, KIN-100, and a linker. A conjugate can comprise an antibody construct, ADU-S100, and a linker.

A conjugate can comprise an antibody construct, a targeting binding domain, an immune-stimulatory compound, and a linker. A conjugate can comprise an antibody construct, a targeting binding domain, a pattern recognition receptor (PRR) agonist, and a linker. A conjugate can comprise an antibody construct, a targeting binding domain, a pattern-associated molecular pattern (PAMP) molecule, and a linker. A conjugate can comprise an antibody construct, a targeting binding domain, a damage-associated molecular pattern (DAMP) molecule, and a linker. A conjugate can comprise an antibody construct, a targeting binding domain, a STING agonist, and a linker. A conjugate can comprise an antibody construct, a targeting binding domain, a toll-like receptor agonist molecule, and a linker. A conjugate can comprise an antibody construct, a targeting binding domain, imiquimod, and a linker. A conjugate can comprise an antibody construct, a targeting binding domain, S-27609, and a linker. A conjugate can comprise an antibody construct, a targeting binding domain, CL307, and a linker. A conjugate can comprise an antibody construct, a targeting binding domain, resiquimod, and a linker. A conjugate can comprise an antibody construct, a targeting binding domain, gardiquimod, and a linker. A conjugate can comprise an antibody construct, a targeting binding domain, UC-IV150, and a linker. A conjugate can comprise an antibody construct, a targeting binding domain, motolimod, and a linker. A conjugate can comprise an antibody construct, a targeting binding domain, VTX-1463, and a linker. A conjugate can comprise an antibody construct, a targeting binding domain, GS-9620, and a linker. A conjugate can comprise an antibody construct, a targeting binding domain, GSK2245035, and a linker. A conjugate can comprise an antibody construct, a targeting binding domain, TMX-101, and a linker. A conjugate can comprise an antibody construct, a targeting binding domain, TMX-201, and a linker. A conjugate can comprise an antibody construct, a targeting binding domain, TMX-202, and a linker. A conjugate can comprise an antibody construct, a targeting binding domain, isatoribine, and a linker. A conjugate can comprise an antibody construct, a targeting binding domain, AZD8848, and a linker. A conjugate can comprise an antibody construct, a targeting binding domain, MEDI9197, and a linker. A conjugate can comprise an antibody construct, a targeting binding domain, 3M-051, and a linker. A conjugate can comprise an antibody construct, a targeting binding domain, 3M-852, and a linker. A conjugate can comprise an antibody construct, a targeting binding domain, 3M-052, and a linker. A conjugate can comprise an antibody construct, a targeting binding domain, 3M-854A, and a linker. A conjugate can comprise an antibody construct, a targeting binding domain, S-34240, and a linker. A conjugate can comprise an antibody construct, a targeting binding domain, CL663, and a linker. A conjugate can comprise an antibody construct, a targeting binding domain, KIN1148, and a linker. A conjugate can comprise an antibody construct, a targeting binding domain, SB-9200, and a linker. A conjugate can comprise an antibody construct, a targeting binding domain, KIN-100, and a linker. A conjugate can comprise an antibody construct, a targeting binding domain, ADU-S100, and a linker.

The linker can be a linker as described herein. A linker can be cleavable, non-cleavable, hydrophilic, or hydrophobic. A cleavable linker can be sensitive to enzymes. A cleavable linker can be cleaved by enzymes such as proteases. A cleavable linker can be a valine-citrulline or a valine-alanine linker. A valine-citrulline or valine-alanine linker can contain a pentafluorophenyl group. A valine-citrulline or valine-alanine linker can contain a succimide group. A valine-citrulline or valine-alanine linker can contain a PABA group. A valine-citrulline or valine-alanine linker can contain a PABA group and a pentafluorophenyl group. A valine-citrulline or valine-alanine linker can contain a PABA group and a succinimide group. A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly)-(NH₂) linker. A linker can be a THIOMAB linker. A THIOMAB linker can be a (maleimidocaproyl)-(valine-citrulline)-(para-aminobenzyloxycarbonly)-(NH₂) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 21) to an N-terminal GGG motif to regenerate a native amide bond. The linker created can therefore link a moiety attached to the LXPTG recognition motif (SEQ ID NO: 21) with a moiety attached to the N-terminal GGG motif. A linker can be a link created between an unnatural amino acid on one moiety reacting with oxime bond that was formed by modifying a ketone group with an alkoxyamine on another moiety. A moiety can be an antibody construct. A moiety can be a targeting binding domain. A moiety can be an antibody. A moiety can be an immune-stimulatory compound.

The antibody construct can be an as described herein. The antibody construct can be an anti-tumor antigen antibody construct. The antibody construct can be an anti-tumor antigen antibody. An antigen recognized by the antibody construct can be CD5, CD19, CD20, CD25, CD37, CD30, CD33, CD45, CAMPATH-1, BCMA, CS-1, PD-L1, B7-H3, B7-DC,HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin, PSCA, MAGE Al, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1. The antibody construct can recognize an antigen that can be expressed on a cell. The antibody construct can recognize an antigen that can be expressed by a cell. The antibody construct can recognize an antigen that can be expressed in the context of a Major Histocompatibility Complex. The antibody construct can recognize an antigen that can stimulate activity of a cell. The antibody construct can recognize an antigen that can stimulate an immune response. The antibody construct can recognize an antigen that can reduce an immune response. The antibody construct can recognize an antigen can reduce activity of a cell. The antibody construct can recognize an antigen that can be expressed on an immune cell. The antibody construct can recognize an antigen that can be expressed by an immune cell. The antibody construct can recognize an antigen that can be in the context of a Major Histocompatibility Complex. The antibody construct can recognize an antigen on a cell wherein the antigen can be involved in stimulating activity of a cell. The antibody construct can recognize an antigen on an immune cell that can be involved in the costimulation of an immune cell. The antibody construct can recognize an antigen on an immune cell that can be involved in the costimulation of an immune cell during an immune response. The antibody construct can recognize a receptor. The antibody construct can recognize a receptor on a cell. The antibody construct can recognize a receptor ligand. The antibody construct can recognize a receptor on a cell wherein the receptor can be involved in stimulating activity of a cell. The antibody construct can recognize a receptor on an immune cell. The antibody construct can recognize a receptor on an immune cell that can be involved in stimulating activity of an immune cell. The antibody construct can recognize a receptor on an immune cell that can be involved in the costimulation of an immune cell. The antibody construct can recognize a receptor on an immune cell that can be involved in the costimulation of an immune cell during an immune response. The antibody construct can recognize an antigen that can be expressed on an immune cell and that can stimulate activity of an immune cell. The antibody construct can recognize an antigen that can be expressed on an immune that can reduce activity of an immune cell. The antibody construct can be an anti-CD40 antibody. The antibody construct can comprise a light chain of an SBT-040 antibody. The antibody construct can comprise an SBT-040-G1WT heavy chain. The antibody construct can comprise an SBT-040-G1VLPLL heavy chain. The antibody construct can comprise an SBT-040-G1DE heavy chain. The antibody construct can comprise an SBT-040-G1AAA heavy chain. The antibody construct can comprise an SBT-040-CDR sequence. The antibody construct can be capable of recognizing a single antigen. The antibody construct can be capable of recognizing two or more antigens. The antibody construct can further comprise a targeting binding domain. An antibody construct can further comprise a targeting binding domain. A targeting binding domain of an antibody construct can recognize an antigen. For example, an antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be CD40 and a targeting binding domain can recognize a CD40 antigen. A targeting binding domain of an antibody construct can be a CD40 agonist.

The antibody construct can have an Fc domain that can bind to an FcR when linked to an immune-stimulatory compound. The antibody construct can have an Fc domain that can bind to an FcR to initiate FcR-mediated signaling when linked to an immune stimulatory compound. The antibody construct can bind to its antigen when linked to an immune-stimulatory compound. The antibody construct can bind to its antigen when linked to an immune-stimulatory compound and the Fc domain of the antibody construct can bind to an FcR when linked to an immune-stimulatory compound. The antibody construct can bind to its antigen when linked to an immune-stimulatory compound and the Fc domain of the antibody can bind to an FcR to initiate FcR-mediated signaling when linked to an immune stimulatory compound.

The targeting binding domain can be selected in order to recognize an antigen. For example, an antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be CD40 and a targeting binding domain can recognize a CD40 antigen. A targeting binding domain can be a CD40 agonist. A targeting binding domain can be CD40.

The PRR agonist can be a toll-like receptor agonist. The toll-like receptor agonist can be a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, a TLR12 agonist or a TLR13 agonist. The toll-like receptor agonist can activate two or more TLRs. The PAMP molecule can be a RIG-I agonist.

A conjugate can be formed by a linker that can connect an antibody construct to a PRR. A conjugate can be formed by a linker that can connect an antibody construct to a PAMP molecule. A conjugate can be formed by a linker that can connect an antibody construct and a DAMP molecule. A conjugate can be formed by a linker that can connect an antibody construct to a PRR, and a linker that can connect an antibody construct and a targeting binding domain. A conjugate can be formed by a linker that can connect an antibody construct to a PAMP molecule, and a linker that can connect an antibody construct and a targeting binding domain. A conjugate can be formed by a linker that can connect an antibody construct and a DAMP molecule, and a linker that can connect an antibody construct and a targeting binding domain.

A linker can be connected to an antibody construct by a direct linkage between the antibody construct and the linker. A linker can be connected to an anti-CD40 antibody construct by a direct linkage between the anti-CD40 antibody construct and the linker. A linker can be connected to an anti-CD40 antibody by a direct linkage between the anti-CD40 antibody and the linker. A linker can be connected to an anti-tumor antigen antibody construct by a direct linkage between the anti-tumor antigen antibody construct and the linker. A linker can be connected to an anti-tumor antigen antibody by a direct linkage between the anti-tumor antigen antibody and the linker. A direct linkage can be a covalent bond. For example, a linker can be attached to a terminus of an amino acid sequence of an antibody construct, or could be attached to a side chain modification to the antibody construct, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. A linker can be connected to an antibody construct at a hinge cysteine. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to antibody construct at an unnatural amino acid engineered into the heavy chain. Amino acids can be engineered into an amino acid sequence of a composition as described herein, for example, a linker of a conjugate. Engineered amino acids can be added to a sequence of existing amino acids. Engineered amino acids can be substituted for one or more existing amino acids of a sequence of amino acids. A linker can be conjugated to antibody construct via a sulfhydryl group. A linker can be conjugated to an antibody construct via a primary amine. A linker can be a link created between an unnatural amino acid on an antibody construct reacting with oxime bond that was formed by modifying a ketone group with an alkoxyamine on an immune-stimulatory compound. When a linker is connected to an antibody construct at the sites described herein, an Fc domain of the antibody construct can bind to Fc receptors. When a linker is connected to an antibody construct at the sites described herein, the antigen binding domain of the antibody construct can bind its antigen. When a linker is connected to an antibody construct at the sites described herein, a targeting binding domain of said antibody construct can bind its antigen.

A linker can connect an antibody construct to a targeting binding domain via THIOMAB linker. A linker can connect an antibody construct to a targeting binding domain via Sortase A linker. A Sortase A linker can be created by a Sortase A enzyme fusing an LXPTG recognition motif (SEQ ID NO: 21) to an N-terminal GGG motif to regenerate a native amide bond. The linker created can therefore link an antibody construct attached the LXPTG recognition motif (SEQ ID NO: 21) with a targeting binding domain attached to the N-terminal GGG motif. A targeting binding domain can be connected to a linker by a direct linkage. A direct linkage can be a covalent bond. For example, a linker can be attached to a terminus of an amino acid sequence of a targeting binding domain, or could be attached to a side chain modification to the targeting binding domain, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the targeting binding domain. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the targeting binding domain. Engineered amino acids can be added to a sequence of existing amino acids. Engineered amino acids can be substituted for one or more existing amino acids of a sequence of amino acids. A linker can be conjugated to a targeting binding domain via a sulfhydryl group. A linker can be conjugated to a targeting binding domain via a primary amine. A targeting binding domain can be conjugated to the C-terminal of an Fc domain of an antibody construct.

A linker can connect an antibody construct to an immune-stimulatory compound via THIOMAB linker. A linker can connect an antibody construct to an immune-stimulatory compound via Sortase A linker. A Sortase A linker can be created by a Sortase A enzyme fusing an LXPTG recognition motif (SEQ ID NO: 21) to an N-terminal GGG motif to regenerate a native amide bond. The linker created can therefore link an antibody construct attached the LXPTG recognition motif (SEQ ID NO: 21) with an immune-stimulatory compound attached to the N-terminal GGG motif. A linker can be a link created between an unnatural amino acid on an antibody construct reacting with oxime bond that was formed by modifying a ketone group with an alkoxyamine on an immune-stimulatory compound. The immune-stimulatory compound can comprise one or more rings selected from carbocyclic and heterocyclic rings. The immune-stimulatory compound can be covalently bound to a linker by a bond to an exocyclic carbon or nitrogen atom on said immune-stimulatory compound. A linker can be conjugated to an immune-stimulatory compound via an exocyclic nitrogen or carbon atom of an immune-stimulatory compound. A linker can be connected to a STING agonist, for example:

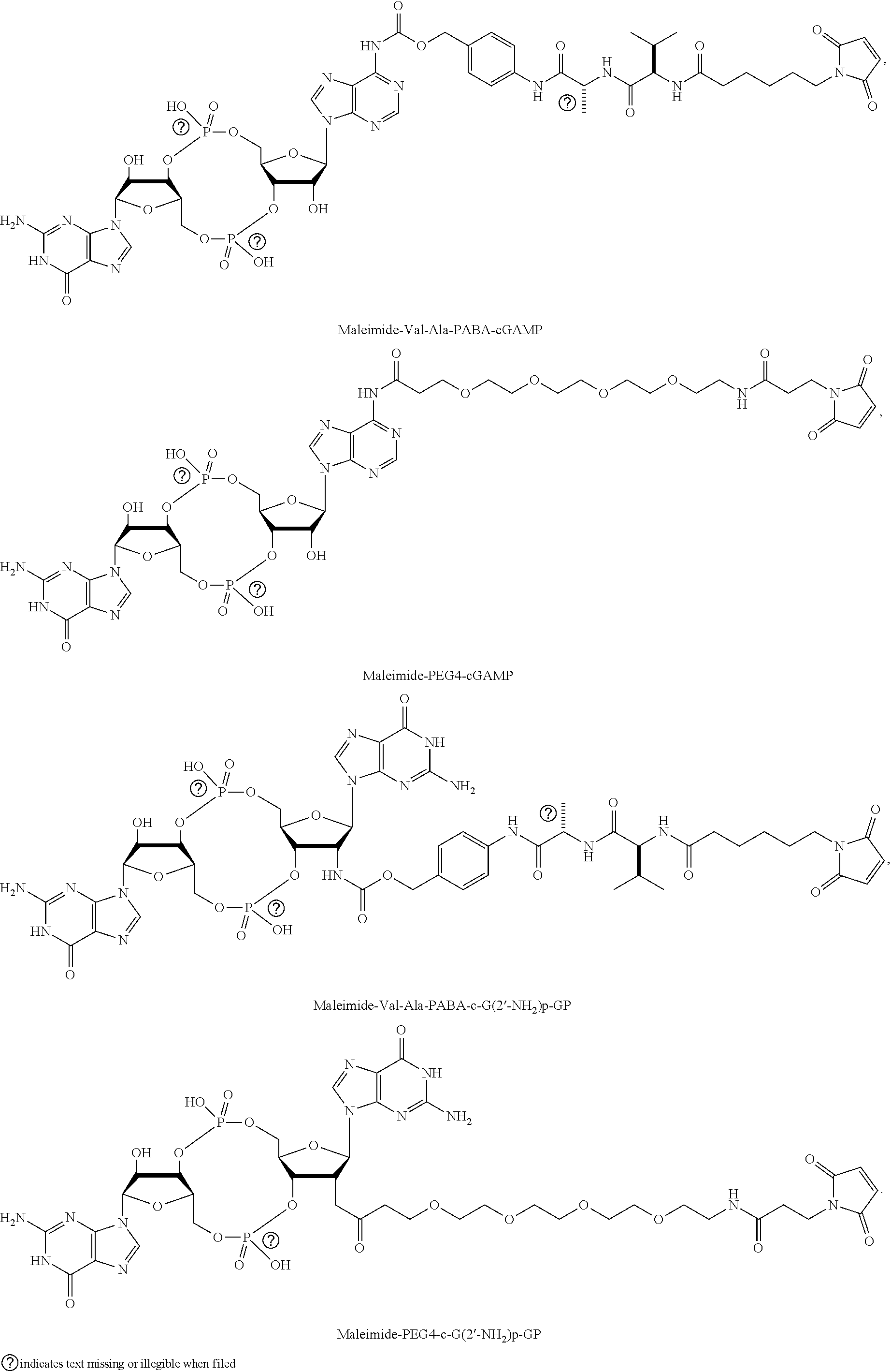

A linker agonist complex can dissociate under physiological conditions to yield an active agonist.

A linker can be connected to a PRR agonist by a direct linkage between the PRR agonist and the linker. A linker can be connected to a PAMP molecule by a direct linkage between the PAMP molecule and the linker. A linker can be connected to a toll-like receptor agonist by a direct linkage between the toll-like receptor agonist and the linker.

Examples of toll-like receptor agonists connected to a linker in a manner able to release an active toll-like receptor agonist under physiologic condition include:

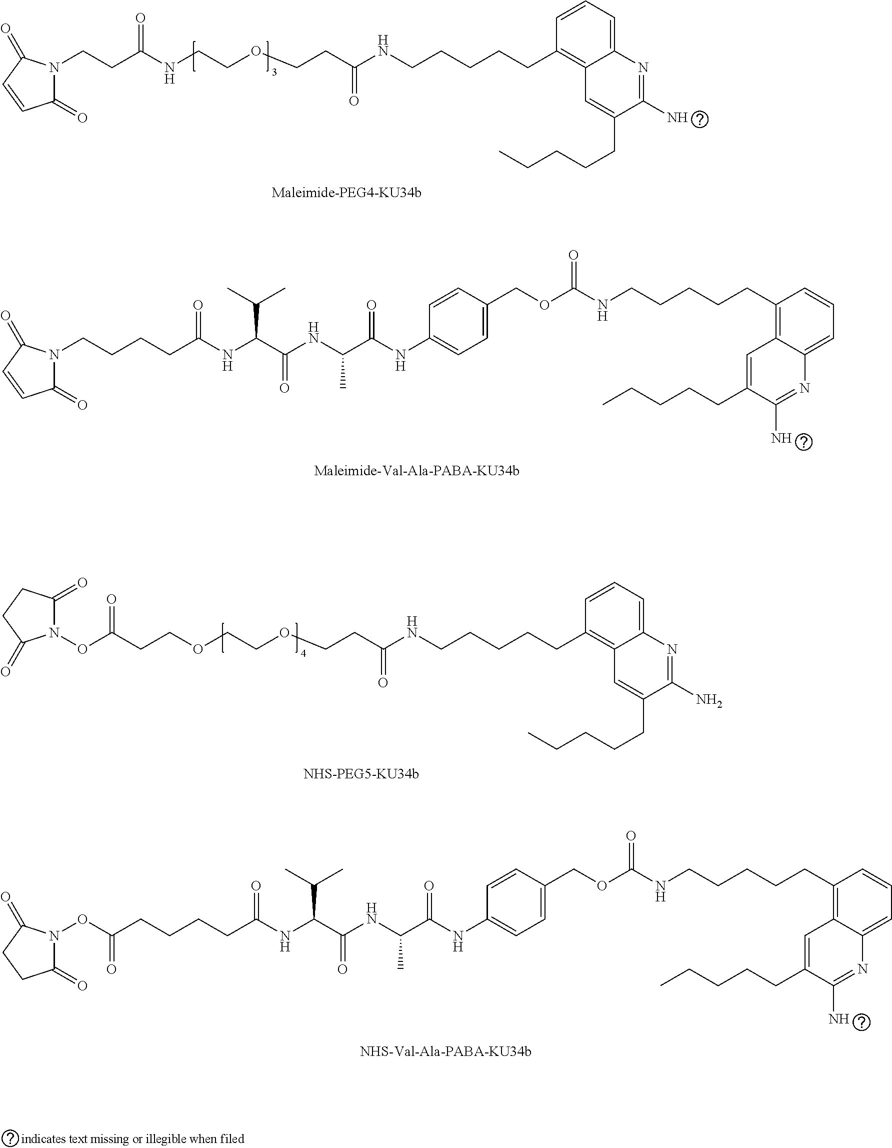

Examples of RIG-1 agonists connected to a linker in a manner able to release an active toll-like receptor agonist under physiologic conditions include:

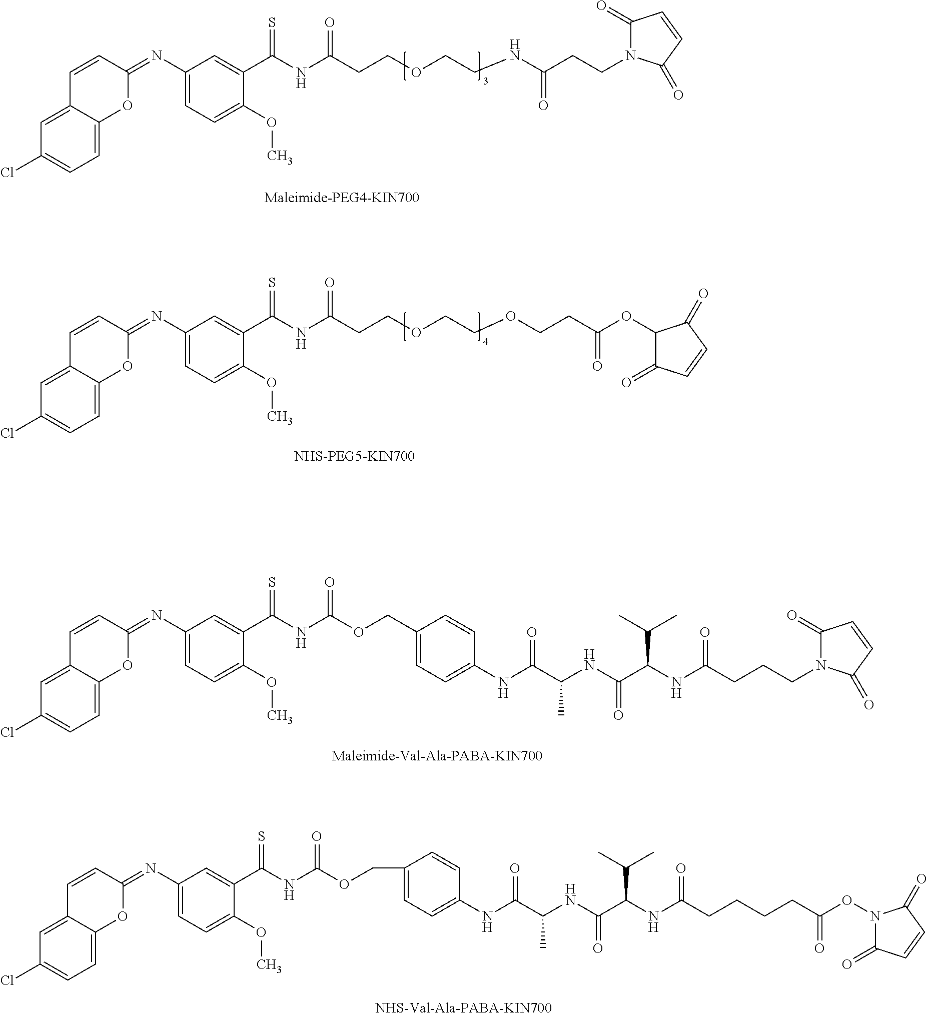

A linker can be connected to a DAMP molecule by a direct linkage between the DAMP molecule and the linker. A direct linkage can be a covalent bond. For example, a linker can be attached to a terminus of an amino acid sequence of an antibody, or could be attached to a side chain modification to the antibody, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody construct. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

An ATAC can be formed by conjugating a noncleavable maleimide-PEG4 linker containing a succinimide group with gardiquimod (ATAC1). An ATAC can be formed by conjugating a cleavable valine-citrulline linker containing a PABA group and a succinimide group with gardiquimod (ATAC2). An ATAC can be formed by conjugating a noncleavable maleimide-PEG4 linker containing a pentafluorophenyl group with gardiquimod (ATAC3). An ATAC can be formed by conjugating a cleavable valine-citrulline linker containing a PABA group and a pentafluorophenyl group with gardiquimod (ATAC4). An ATAC can be formed by conjugating a noncleavable maleimide-PEG4 linker containing a succinimide group with UC-1V150 (ATAC5). An ATAC can be formed by conjugating a cleavable valine-citrulline linker containing a PABA group and a succinimide group with UC-1V150 (ATAC6). An ATAC can be formed by conjugating a noncleavable maleimide-PEG4 linker containing a pentafluorophenyl group with UC-1V150 (ATAC7). An ATAC can be formed by conjugating a cleavable valine-citrulline linker containing a PABA group and a pentafluorophenyl group with UC-1V150 (ATAC8). An ATAC can be formed by conjugating a noncleavable maleimide-PEG4 linker containing a succinimide group with KU34B (ATAC9). An ATAC can be formed by conjugating a cleavable valine-citrulline linker containing a PABA group and a succinimide group with KU34B (ATAC10). An ATAC can be formed by conjugating a noncleavable maleimide-PEG4 linker containing a pentafluorophenyl group with KU34B (ATAC11). An ATAC can be formed by conjugating a cleavable valine-citrulline linker containing a PABA group and a pentafluorophenyl group with KU34B (ATAC12).

An anti-CD40 antibody can comprise two SBT-040-G1WT heavy chains and two light chain from a SBT-040 antibody, which can be referred to as SBT-040-WT. An anti-CD40 antibody can comprise two SBT-040-G1VLPLL heavy chains and two light chains from a SBT-040 antibody, which can be referred to as SBT-040-VLPLL. An anti-CD40 antibody can comprise two SBT-040-G1DE heavy chains and two light chains from a SBT-040 antibody, which can be referred to as SBT-040-DE. An anti-CD40 antibody can comprise two SBT-040-G1AAA heavy chains and two light chains from a SBT-040 antibody, which can be referred to as SBT-040-AAA.

A conjugate can comprise SBT-040-WT-ATAC1. A conjugate can comprise SBT-040-WT-ATAC2. A conjugate can comprise SBT-040-WT-ATAC3. A conjugate can comprise SBT-040-WT-ATAC4. A conjugate can comprise SBT-040-WT-ATAC5. A conjugate can comprise SBT-040-WT-ATAC6. A conjugate can comprise SBT-040-WT-ATAC7. A conjugate can comprise SBT-040-WT-ATAC8. A conjugate can comprise SBT-040-WT-ATAC9. A conjugate can comprise SBT-040-WT-ATAC10. A conjugate can comprise SBT-040-WT-ATAC11. A conjugate can comprise SBT-040-WT-ATAC12. A conjugate can comprise SBT-040-VLPLL-ATAC1. A conjugate can comprise SBT-040-VLPLL-ATAC2. A conjugate can comprise SBT-040-VLPLL-ATAC3. A conjugate can comprise SBT-040-VLPLL-ATAC4. A conjugate can comprise SBT-040-VLPLL-ATAC5. A conjugate can comprise SBT-040-VLPLL-ATAC6. A conjugate can comprise SBT-040-VLPLL-ATAC7. A conjugate can comprise SBT-040-VLPLL-ATAC8. A conjugate can comprise SBT-040-VLPLL-ATAC9. A conjugate can comprise SBT-040-VLPLL-ATAC10. A conjugate can comprise SBT-040-VLPLL-ATAC11. A conjugate can comprise SBT-040-VLPLL-ATAC12. A conjugate can comprise SBT-040-DE-ATAC1. A conjugate can comprise SBT-040-DE-ATAC2. A conjugate can comprise SBT-040-DE-ATAC3. A conjugate can comprise SBT-040-DE-ATAC4. A conjugate can comprise SBT-040-DE-ATAC5. A conjugate can comprise SBT-040-DE-ATAC6. A conjugate can comprise SBT-040-DE-ATAC7. A conjugate can comprise SBT-040-DE-ATAC8. A conjugate can comprise SBT-040-DE-ATAC9. A conjugate can comprise SBT-040-DE-ATAC10. A conjugate can comprise SBT-040-DE-ATAC11. A conjugate can comprise SBT-040-DE-ATAC12. A conjugate can comprise SBT-040-AAA-ATAC1. A conjugate can comprise SBT-040-AAA-ATAC2. A conjugate can comprise SBT-040-AAA-ATAC3. A conjugate can comprise SBT-040-AAA-ATAC4. A conjugate can comprise SBT-040-AAA-ATAC5. A conjugate can comprise SBT-040-AAA-ATAC6. A conjugate can comprise SBT-040-AAA-ATAC7. A conjugate can comprise SBT-040-AAA-ATAC8. A conjugate can comprise SBT-040-AAA-ATAC9. A conjugate can comprise SBT-040-AAA-ATAC10. A conjugate can comprise SBT-040-AAA-ATAC11. A conjugate can comprise SBT-040-AAA-ATAC12.

In a conjugate, an antibody conjugate can be linked to an immune-stimulatory compound in such a way that the antibody can still bind to an antigen and the Fc domain of the antibody can still bind to an FcR. In a conjugate, an antibody construct is linked to an immune-stimulatory compound in such a way that the linking does not interfere with ability of the antibody to bind to antigen, the ability of the Fc domain of the antibody to bind to an FcR, or FcR-mediated signaling resulting from the Fc domain of the antibody from binding to an FcR. In a conjugate, an immune-stimulatory compound can be linked to an antibody construct in such a way the linking does not interfere with the ability of the immune-stimulatory compound to bind to its receptor. A conjugate can produce stronger immune stimulation and a greater therapeutic window than components of the conjugate alone. In an anti-CD40 antibody linked to a TLR agonist conjugate, the combination of CD40 agonism, TLR agonism, and an accessible Fc domain of the anti-CD40 antibody to allow FcR-mediated signaling can produce stronger immune stimulation and a greater therapeutic window than the CD40 agonism, TLR agonism, or the FcR-mediated signaling alone.

### Pharmaceutical Formulations

The compositions and methods described herein can be considered useful as pharmaceutical compositions for administration to a subject in need thereof. Pharmaceutical compositions can comprise at least the compositions described herein and one or more pharmaceutically acceptable carriers, diluents, excipients, stabilizers, dispersing agents, suspending agents, and/or thickening agents. The composition can comprise the conjugate having an antibody construct and an agonist. The composition can comprise the conjugate having an antibody construct, a targeting binding domain, and an agonist. The composition can comprise any conjugate described herein. Often, the antibody construct is an anti-CD40 antibody. A conjugate can comprise an anti-CD40 antibody and a PAMP molecule. A conjugate can comprise an anti-CD40 antibody and a DAMP molecule. A pharmaceutical composition can further comprise buffers, antibiotics, steroids, carbohydrates, drugs (e.g., chemotherapy drugs), radiation, polypeptides, chelators, adjuvants and/or preservatives.

Pharmaceutical compositions can be formulated using one or more physiologically-acceptable carriers comprising excipients and auxiliaries. Formulation can be modified depending upon the route of administration chosen. Pharmaceutical compositions comprising a composition as described herein can be manufactured, for example, by lyophilizing the conjugate, mixing, dissolving, emulsifying, encapsulating or entrapping the conjugate. The pharmaceutical compositions can also include the compositions described herein in a free-base form or pharmaceutically-acceptable salt form.

Methods for formulation of the conjugates described herein can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use

Pharmaceutical compositions of the conjugates described herein can comprise at least an active ingredient. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the composition can comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished by filtration through sterile filtration.

The compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration the conjugates can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicles can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can be also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The compositions and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions and methods described herein can be useful as a therapeutic, for example a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

A therapeutically effective amount can be the amount of a composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates described herein that can be used in therapy can be formulated and dosages established in a fashion consistent with good medical practice taking into account the disorder to be treated, the condition of the individual patient, the site of delivery of the composition, the method of administration and other factors known to practitioners. The conjugates described herein can be prepared according to the description of preparation described herein.

Pharmaceutical compositions can be considered useful with the compositions and methods described herein can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods and compositions described herein can be for administration to a subject in need thereof. Often, administration of the compositions described herein can include routes of administration, non-limiting examples of administration routes include intravenous, intraarterial, subcutaneous, subdural, intramuscular, intrancranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month.

### Diseases, Conditions and the Like

The compositions and methods provided herein can be useful for the treatment of a plurality of diseases, conditions, preventing a disease or a condition in a subject or other therapeutic applications for subjects in need thereof. Often the compositions and methods provided herein can be useful for treatment of hyperplastic conditions, including but not limited to, neoplasms, cancers, tumors and the like. A condition, such as a cancer, can be associated with expression of a molecule on the cancer cells. Often, the molecule expressed by the cancer cells can comprise an extracellular portion capable of recognition by the antibody portion of the conjugate. A molecule expressed by the cancer cells can be a tumor antigen. An antibody portion of the conjugate can recognize a tumor antigen. A tumor antigen can include CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin, PSCA, MAGE Al, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1.

As described herein, an antigen binding domain portion of the conjugate, can be configured to recognize a molecule expressed by a cancer cell, such as for example, a disease antigen, tumor antigen or a cancer antigen. Often such antigens that are known to those of ordinary skill in the art, or newly found to be associated with such a condition, to be commonly associated with, and/or, specific to, such conditions. For example, a disease antigen, tumor antigen or a cancer antigen is, but is not limited to, CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin, PSCA, MAGE Al, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1. Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma,; Thyroid cancer; Thyroid cancer, childhood; Uterine cancer; Vaginal cancer; Waldenström macroglobulinemia; Wilms tumor and any combination thereof.

### EXAMPLE 1

### Fc Receptor Binding to Anti-CD40 Antibodies

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are produced by standard methods for producing antibodies. These antibodies are purified, and each antibody's affinity for soluble glycosylated ectodomains from all human Fcγ receptors (FcγRs) is measured. These affinities are measured by experiments using surface plasmon resonance. In these experiments, biotinylated soluble glycosylated FcγR ectodomains from all human FcγRs are immobilized on a streptavidin-coated surface. The ability of each antibody to bind to soluble glycosylated FcγR ectodomains from all human FcγRs is then measured by surface plasmon resonance using a Biacore instrument. The data from this experiment shows that the Fc domain of a SBT-040-WT antibody, the Fc domain of a SBT-040-VLPLL antibody, the Fc domain of a SBT-040-DE antibody, and the Fc domain of a SBT-040-AAA antibody are each bound to soluble glycosylated FcγR ectodomains from all human FcγRs. Therefore, the surface plasmon resonance experiments show that the Fc domain of the SBT-040-G1WT antibody and variants of the Fc domain of a SBT-040-G1WT antibody (i.e., the Fc domain of a SBT-040-G1VLPLL antibody, the Fc domain of a SBT-040-DE antibody and the Fc domain of a SBT-040-AAA antibody) are each bound to all human FcγRs. The affinity of each antibody for each human FcγRs is also shown by these experiments.

### EXAMPLE 2

### Synthesis of Linkers with Immune-Stimulatory Compounds

A linker is linked with an immune-stimulatory compound. A linker linked to an immune-stimulatory compound is formed to make a linker-immune stimulatory compound conjugate (ATAC). Subsequently, an ATAC is conjugated to an antibody.

A linker is a valine-citrulline linker. A linker is an N-Maleimidomethylcyclohexane-1-carboxylate (MCC) linker. An immune-stimulatory compound is gardiquimod. An immune-stimulatory compound is UC-1V150.

An ATAC is formed by conjugating a noncleavable maleimide-PEG4 linker containing a succinimide group with gardiquimod (ATAC1). An ATAC is formed by conjugating a cleavable valine-citrulline linker containing a PABA group and a succinimide group with gardiquimod (ATAC2). An ATAC is formed by conjugating a noncleavable maleimide-PEG4 linker containing a pentafluorophenyl group with gardiquimod (ATAC3). An ATAC is formed by conjugating a cleavable valine-citrulline linker containing a PABA group and a pentafluorophenyl group with gardiquimod (ATAC4). An ATAC is formed by conjugating a noncleavable maleimide-PEG4 linker containing a succinimide group with UC-1V150 (ATAC5). An ATAC is formed by conjugating a cleavable valine-citrulline linker containing a PABA group and a succinimide group with UC-1V150 (ATAC6). An ATAC is formed by conjugating a noncleavable maleimide-PEG4 linker containing a pentafluorophenyl group with UC-1V150 (ATAC7). An ATAC is formed by conjugating a cleavable valine-citrulline linker containing a PABA group and a pentafluorophenyl group with UC-1V150 (ATAC8). An ATAC is formed by conjugating a noncleavable maleimide-PEG4 linker containing a succinimide group with KU34B (ATAC9). An ATAC is formed by conjugating a cleavable valine-citrulline linker containing a PABA group and a succinimide group with KU34B (ATAC10). An ATAC is formed by conjugating a noncleavable maleimide-PEG4 linker containing a pentafluorophenyl group with KU34B (ATAC11). An ATAC is formed by conjugating a cleavable valine-citrulline linker containing a PABA group and a pentafluorophenyl group with KU34B (ATAC12).

### EXAMPLE 3

### Fc Receptor Binding to Anti-CD40 Antibody Immunoactivator Conjugates

An anti-CD40 antibody is comprised of two SBT-040-G1WT heavy chains and two light chains from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

Each antibody is purified and then each is conjugated to ATAC1, ATAC2, ATAC3, ATAC4, ATAC5, ATAC6, ATAC7, or ATAC8. ATAC1, ATAC2, ATAC3, ATAC4, ATAC5, ATAC6, ATAC7, ATAC8, ATAC9, ATAC10, ATAC11, and ATAC12 as described in Example 2. Each of these conjugates is characterized for the ability of their Fc domains to bind to and for their affinity for soluble glycosylated FcγR ectodomains from all human FcγRs. This is shown by performing surface plasmon resonance experiments. In these experiments, biotinylated soluble glycosylated FcγR ectodomains from all human FcγRs are immobilized on a streptavidin-coated surface. The ability of each conjugate to bind to soluble glycosylated FcγR ectodomains from all human FcγRs is then measured by surface plasmon resonance using a Biacore instrument. The data from these experiments shows that the Fc domain of the SBT-040-WT-ATAC1 conjugate, the Fc domain of the SBT-040-WT-ATAC2 conjugate, the Fc domain of the SBT-040-WT-ATAC3 conjugate, the Fc domain of the SBT-040-WT-ATAC4 conjugate, the Fc domain of the SBT-040-WT-ATAC5 conjugate, the Fc domain of the SBT-040-WT-ATAC6 conjugate, the Fc domain of the SBT-040-WT-ATAC7 conjugate, the Fc domain of the SBT-040-WT-ATAC8 conjugate, the Fc domain of the SBT-040-WT-ATAC9 conjugate, the Fc domain of the SBT-040-WT-ATAC10 conjugate, the Fc domain of the SBT-040-WT-ATAC11 conjugate, the Fc domain of the SBT-040-WT-ATAC12 conjugate, the Fc domain of the SBT-040-VLPLL-ATAC1 conjugate, the Fc domain of the SBT-040-VLPLL-ATAC2 conjugate, the Fc domain of the SBT-040-VLPLL-ATAC3 conjugate, the Fc domain of the SBT-040-VLPLL-ATAC4 conjugate, the Fc domain of the SBT-040-VLPLL-ATAC5 conjugate, the Fc domain of the SBT-040-VLPLL-ATAC6 conjugate, the Fc domain of the SBT-040-VLPLL-ATAC7 conjugate, the Fc domain of the SBT-040-VLPLL-ATAC8 conjugate, the Fc domain of the SBT-040-VLPLL-ATAC9 conjugate, the Fc domain of the SBT-040-VLPLL-ATAC10 conjugate, the Fc domain of the SBT-040-VLPLL-ATAC11 conjugate, the Fc domain of the SBT-040-VLPLL-ATAC12 conjugate, the Fc domain of the SBT-040-DE-ATAC1 conjugate, the Fc domain of the SBT-040-DE-ATAC2 conjugate, the Fc domain of the SBT-040-DE-ATAC3 conjugate, the Fc domain of the SBT-040-DE-ATAC4 conjugate, the Fc domain of the SBT-040-DE-ATAC5 conjugate, the Fc domain of the SBT-040-DE-ATAC6 conjugate, the Fc domain of the SBT-040-DE-ATAC7 conjugate, the Fc domain of the SBT-040-DE-ATAC8 conjugate, the Fc domain of the SBT-040-DE-ATAC9 conjugate, the Fc domain of the SBT-040-DE-ATAC10 conjugate, the Fc domain of the SBT-040-DE-ATAC11 conjugate, the Fc domain of the SBT-040-DE-ATAC12 conjugate, the Fc domain of the SBT-040-AAA-ATAC1 conjugate, the Fc domain of the SBT-040-AAA-ATAC2 conjugate, the Fc domain of the SBT-040-AAA-ATAC3 conjugate, the Fc domain of the SBT-040-AAA-ATAC4 conjugate, the Fc domain of the SBT-040-AAA-ATAC5 conjugate, the Fc domain of the SBT-040-AAA-ATAC6 conjugate, the Fc domain of the SBT-040-AAA-ATAC7 conjugate, and the Fc domain of the SBT-040-AAA-ATAC8 conjugate, the Fc domain of the SBT-040-AAA-ATAC9 conjugate, the Fc domain of the SBT-040-AAA-ATAC10 conjugate, the Fc domain of the SBT-040-AAA-ATAC11 conjugate, and the Fc domain of the SBT-040-AAA-ATAC12 conjugate are all bound to soluble glycosylated FcγR ectodomains from all human FcγRs. Therefore, the surface plasmon resonance experiments show that the ability of the Fc domain of the antibody component of the conjugate to bind to all human FcγRs is not interfered with by the conjugation of the components of the conjugate. The affinity of each conjugate for each human FcγRs is also shown by the surface plasmon resonance experiments. These affinity measurements are compared with the affinity measurements for each antibody alone (as can be shown by Example 1). The similarity in affinity of each antibody alone for soluble glycosylated FcγR ectodomains from all human FcγRs with the affinity of each corresponding conjugate for soluble glycosylated FcγR ectodomains from all human FcγRs is shown by this comparison.

### EXAMPLE 4

### Affinity of Anti-CD40 Antibodies to CD40

An anti-CD40 antibody is comprised of two SBT-040-G1WT heavy chains and two light chain from a SBT-040 antibody, which are referred to as a SBT-040-WT antibody. An anti-CD40 antibody is comprised of two SBT-040-G1VLPLL heavy chains and two light chains from a SBT-040 antibody, which are referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are each produced by standard methods for producing antibodies. Each antibody is purified, and then is characterized for the ability to bind to CD40. This characterization is shown by experiments using flow cytometry. For these experiments, the human Burkitt's Lymphoma tumor cell lines Raji and Daudi, which are previously shown to be CD40-positive, and the human Chronic Myelogenous Leukemia tumor cell line K562, which is previously shown to be CD40-negative are first evaluated by flow cytometry to assess their relative expression levels of CD40. This is assessed by incubating each cell line with a commercially available CD40 antibody conjugated to a fluorochrome, and then running samples of the incubation on a flow cytometer. The relative fluorescent intensity profiles for each cell line is shown by this data, indicating the level of CD40 expression of each cell line. The relative fluorescent intensity profiles of human Burkitt's Lymphoma tumor cell lines Raji and Daudi show that CD40 is expressed in each of these cell lines, whereas the relative fluorescent intensity profile of the human Chronic Myelogenous Leukemia tumor cell line K562 show that CD40 is not expressed in the cell line. Then, each cell line is separately incubated with purified SBT-040-WT antibody, SBT-040-VLPLL antibody, SBT-040G1DE antibody, SBT-040-AAA antibody or no antibody as a control. Each incubation is further incubated with a secondary anti-human IgG1 antibody conjugated with FITC, which is then each assessed by flow cytometry for the FITC fluorescent intensity profile of each sample. The ability of each antibody to detect CD40 expression on the cell lines is indicated by their FITC fluorescent intensity profile. More specifically, the similarity between the SBT-040-WT antibody fluorescent intensity profile and each antibody with an Fc-enhanced IgG1 isotype after incubation with each of the cell lines is shown by this data. Each Fc-enhanced IgG1 isotype is not altered by the ability of the antibody to bind to CD40-positive cells is also shown by this data.

### EXAMPLE 5

### Affinity of Anti-CD40 Antibodies to CD40

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised two SBT-040-G1DE heavy chains and two light chains from a SBT-040 antibody, which is referred to as a SBT-040-DE antibody. An anti-CD40 antibody is comprised two SBT-040-G1AAA heavy chains and two light chains from a SBT-040 antibody, which is referred to as a SBT-040-AAA antibody.

SBT-040-WT antibody, SBT-040-G1VLPLL antibody, SBT-040-DE antibody, and SBT-040-AAA antibody are each produced by standard methods for producing antibodies. Each antibody is purified, and each antibody's affinity for CD40 is measured. These affinities are measured by experiments using surface plasmon resonance. In these experiments, biotinylated recombinant CD40 is immobilized on a streptavidin-coated surface. The ability of each antibody to bind to recombinant CD40 is then measured by surface plasmon resonance using a Biacore instrument. The data from these experiments shows that SBT-040-WT antibody, SBT-040-VLPLL antibody, SBT-040-DE antibody, and SBT-040-AAA antibody are each bound to recombinant CD40. Therefore, each antibody's ability to bind to CD40 is not interfered with by the enhanced Fc-enhanced IgG1 isotypes is shown by the surface plasmon resonance data.

Furthermore, surface plasmon resonance is used to show that CD40L binding to CD40 is not blocked by these antibodies. In these experiments, biotinylated recombinant CD40 is immobilized on a streptavidin-coated surface. Surface plasmon resonance using a Biacore instrument is then used to measure the binding affinity of CD40L in the presence of each antibody or without any antibody as a control. The binding affinity of CD40L with recombinant CD40 in presence of each antibody is shown to be the same as the binding affinity of the CD40L with recombinant CD40 in the absence of any antibody. Therefore, CD40 and CD40L binding is unaffected by the presence of SBT-040-WT antibody, SBT-040-G1VLPLL antibody, SBT-040-DE antibody, or SBT-040-AAA antibody.

### EXAMPLE 6

### Fc Receptor Binding to Anti-CD40 Antibody Immunoactivator Conjugates

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-G1VLPLL antibody, SBT-040-DE antibody, and SBT-040-AAA antibody are each made following standard methods for antibody production. Each antibody is purified and then each is conjugated to ATAC1, ATAC2, ATAC3, ATAC4, ATAC5, ATAC6, ATAC7, or ATAC8. ATAC1, ATAC2, ATAC3, ATAC4, ATAC5, ATAC6, ATAC7, ATAC8, ATAC9, ATAC10, ATAC, 11, and ATAC12 are as described in Example 2. The affinity of each conjugate for CD40 is then measured by experiments using surface plasmon resonance. In these experiments, biotinylated recombinant CD40 is immobilized on a streptavidin-coated surface. The ability of each conjugate to bind to recombinant CD40 is then measured by surface plasmon resonance using a Biacore instrument. The data from these experiments shows that the SBT-040-WT-ATAC1 conjugate, the SBT-040-WT-ATAC2 conjugate, the SBT-040-WT-ATAC3 conjugate, the SBT-040-WT-ATAC4 conjugate, the SBT-040-WT-ATAC5 conjugate, the SBT-040-WT-ATAC6 conjugate, the SBT-040-WT-ATAC7 conjugate, the SBT-040-WT-ATAC8 conjugate, the SBT-040-WT-ATAC9 conjugate, the SBT-040-WT-ATAC10 conjugate, the SBT-040-WT-ATAC11 conjugate, the SBT-040-WT-ATAC12 conjugate, the SBT-040-VLPLL-ATAC1 conjugate, the SBT-040-VLPLL-ATAC2 conjugate, the SBT-040-VLPLL-ATAC3 conjugate, the SBT-040-VLPLL-ATAC4 conjugate, the SBT-040-VLPLL-ATAC5 conjugate, the SBT-040-VLPLL-ATAC6 conjugate, the SBT-040-VLPLL-ATAC7 conjugate, the SBT-040-VLPLL-ATAC8 conjugate, the SBT-040-VLPLL-ATAC9 conjugate, the SBT-040-VLPLL-ATAC10 conjugate, the SBT-040-VLPLL-ATAC11 conjugate, the SBT-040-VLPLL-ATAC12 conjugate, the SBT-040-DE-ATAC1 conjugate, the SBT-040-DE-ATAC2 conjugate, the SBT-040-DE-ATAC3 conjugate, the SBT-040-DE-ATAC4 conjugate, the SBT-040-DE-ATAC5 conjugate, the SBT-040-DE-ATAC6 conjugate, the SBT-040-DE-ATAC7 conjugate, the SBT-040-DE-ATAC8 conjugate, the SBT-040-DE-ATAC9 conjugate, the SBT-040-DE-ATAC10 conjugate, the SBT-040-DE-ATAC11 conjugate, the SBT-040-DE-ATAC12 conjugate, the SBT-040-AAA-ATAC1 conjugate, the SBT-040-AAA-ATAC2 conjugate, the SBT-040-AAA-ATAC3 conjugate, the SBT-040-AAA-ATAC4 conjugate, the SBT-040-AAA-ATAC5 conjugate, the SBT-040-AAA-ATAC6 conjugate, the SBT-040-AAA-ATAC7 conjugate, and the SBT-040-AAA-ATAC8, the SBT-040-AAA-ATAC9 conjugate, the SBT-040-AAA-ATAC10 conjugate, the SBT-040-AAA-ATAC11 conjugate, and the SBT-040-AAA-ATAC12 conjugate are all bound to recombinant CD40. Therefore, the surface plasmon resonance experiments show that each component antibody's ability to bind to CD40 is not interfered with by the enhanced Fc-enhanced IgG1 isotypes nor the antibody conjugation to ATAC1, ATAC2, ATAC3, ATAC4, ATAC5, ATAC6, ATAC7, ATAC8, ATAC9, ATAC10, ATAC11, or ATAC12.

Furthermore, surface plasmon resonance is used to show that CD40L binding to CD40 is not blocked in the presence of each conjugate. In these experiments, biotinylated recombinant CD40 is immobilized on a streptavidin-coated surface. Surface plasmon resonance using a Biacore instrument is then used to measure the binding affinity of CD40L in the presence of each conjugate or without any conjugate as a control. The binding affinity of CD40L with recombinant CD40 in presence of each conjugate is shown to be the same as the binding affinity of the CD40L with recombinant CD40 in the absence of any conjugate by these experiments. Therefore, CD40 and CD40L binding is unaffected by the presence of the SBT-040-WT-ATAC1 conjugate, the SBT-040-WT-ATAC2 conjugate, the SBT-040-WT-ATAC3 conjugate, the SBT-040-WT-ATAC4 conjugate, the SBT-040-WT-ATAC5 conjugate, the SBT-040-WT-ATAC6 conjugate, the SBT-040-WT-ATAC7 conjugate, the SBT-040-WT-ATAC8 conjugate, the SBT-040-WT-ATAC9 conjugate, the SBT-040-WT-ATAC10 conjugate, the SBT-040-WT-ATAC11 conjugate, the SBT-040-WT-ATAC12 conjugate, the SBT-040-VLPLL-ATAC1 conjugate, the SBT-040-VLPLL-ATAC2 conjugate, the SBT-040-VLPLL-ATAC3 conjugate, the SBT-040-VLPLL-ATAC4 conjugate, the SBT-040-VLPLL-ATAC5 conjugate, the SBT-040-VLPLL-ATAC6 conjugate, the SBT-040-VLPLL-ATAC7 conjugate, the SBT-040-VLPLL-ATAC8 conjugate, the SBT-040-DE-ATAC1 conjugate, the SBT-040-VLPLL-ATAC9 conjugate, the SBT-040-VLPLL-ATAC10 conjugate, the SBT-040-VLPLL-ATAC11 conjugate, the SBT-040-VLPLL-ATAC12 conjugate, the SBT-040-DE-ATAC1 conjugate, the SBT-040-DE-ATAC2 conjugate, the SBT-040-DE-ATAC3 conjugate, the SBT-040-DE-ATAC4 conjugate, the SBT-040-DE-ATAC5 conjugate, the SBT-040-DE-ATAC6 conjugate, the SBT-040-DE-ATAC7 conjugate, the SBT-040-DE-ATAC8 conjugate, the SBT-040-DE-ATAC9 conjugate, the SBT-040-DE-ATAC10 conjugate, the SBT-040-DE-ATAC11 conjugate, the SBT-040-DE-ATAC12 conjugate, the SBT-040-AAA-ATAC1 conjugate, the SBT-040-AAA-ATAC2 conjugate, the SBT-040-AAA-ATAC3 conjugate, the SBT-040-AAA-ATAC4 conjugate, the SBT-040-AAA-ATAC5 conjugate, the SBT-040-AAA-ATAC6 conjugate, the SBT-040-AAA-ATAC7 conjugate, or the SBT-040-AAA-ATAC8 conjugate, the SBT-040-AAA-ATAC9 conjugate, the SBT-040-AAA-ATAC10 conjugate, the SBT-040-AAA-ATAC11 conjugate, or the SBT-040-AAA-ATAC12 conjugate.

### EXAMPLE 7

### Cytokine Production is Enhanced by Anti-CD40 Antibody Immunoactivator Conjugates

Antibody-immune stimulatory compound conjugates are enhanced by cytokine production when co-cultured with dendritic cells. In this experiment, dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSG. The non-adherent cells are isolated and are washed. These isolated cells are run by a flow cytometer to ensure CD11c expression, in which the DCs identity as DCs is confirmed by CD11c expression. The DCs are then incubated with either the antibodies as described in Example 1 or the conjugates as described in Example 3. More specifically, the DCs are incubated with SBT-040-WT antibody, SBT-040-VLPLL antibody, SBT-040-DE antibody, SBT-040-AAA antibody, the SBT-040-WT-ATAC1 conjugate, the SBT-040-WT-ATAC2 conjugate, the SBT-040-WT-ATAC3 conjugate, the SBT-040-WT-ATAC4 conjugate, the SBT-040-WT-ATAC5 conjugate, the SBT-040-WT-ATAC6 conjugate, the SBT-040-WT-ATAC7 conjugate, the SBT-040-WT-ATAC8 conjugate, the SBT-040-VLPLL-ATAC1 conjugate, the SBT-040-WT-ATAC9 conjugate, the SBT-040-WT-ATAC10 conjugate, the SBT-040-WT-ATAC11 conjugate, the SBT-040-WT-ATAC12 conjugate, the SBT-040-VLPLL-ATAC1 conjugate, the SBT-040-VLPLL-ATAC2 conjugate, the SBT-040-VLPLL-ATAC3 conjugate, the SBT-040-VLPLL-ATAC4 conjugate, the SBT-040-VLPLL-ATAC5 conjugate, the SBT-040-VLPLL-ATAC6 conjugate, the SBT-040-VLPLL-ATAC7 conjugate, the SBT-040-VLPLL-ATAC8 conjugate, the SBT-040-VLPLL-ATAC9 conjugate, the SBT-040-VLPLL-ATAC10 conjugate, the SBT-040-VLPLL-ATAC11 conjugate, the SBT-040-VLPLL-ATAC12 conjugate, the SBT-040-DE-ATAC1 conjugate, the SBT-040-DE-ATAC2 conjugate, the SBT-040-DE-ATAC3 conjugate, the SBT-040-DE-ATAC4 conjugate, the SBT-040-DE-ATAC5 conjugate, the SBT-040-DE-ATAC6 conjugate, the SBT-040-DE-ATAC7 conjugate, the SBT-040-DE-ATAC8 conjugate, the SBT-040-DE-ATAC9 conjugate, the SBT-040-DE-ATAC10 conjugate, the SBT-040-DE-ATAC11 conjugate, the SBT-040-DE-ATAC12 conjugate, the SBT-040-AAA-ATAC1 conjugate, the SBT-040-AAA-ATAC2 conjugate, the SBT-040-AAA-ATAC3 conjugate, the SBT-040-AAA-ATAC4 conjugate, the SBT-040-AAA-ATAC5 conjugate, the SBT-040-AAA-ATAC6 conjugate, the SBT-040-AAA-ATAC7 conjugate, the SBT-040-AAA-ATAC8 conjugate, the SBT-040-AAA-ATAC9 conjugate, the SBT-040-AAA-ATAC10 conjugate, the SBT-040-AAA-ATAC11 conjugate, the SBT-040-AAA-ATAC12 conjugate or a non-binding isotype control antibody. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The supernatant from the culture containing the non-binding isotype control shows the level of cytokine expression is decreased as compared to the supernatant from cultures containing SBT-040-WT, SBT-040-VLPLL, SBT-040-DE, or SBT-040-AAA. Additionally, the level of cytokine expression in the supernatant from cultures containing SBT-040-WT, SBT-040-G1VLPLL, SBT-040-DE, or SBT-040-AAA is decreased as compared to the supernatant from cultures containing the SBT-040-WT-ATAC1 conjugate, the SBT-040-WT-ATAC2 conjugate, the SBT-040-WT-ATAC3 conjugate, the SBT-040-WT-ATAC4 conjugate, the SBT-040-WT-ATAC5 conjugate, the SBT-040-WT-ATAC6 conjugate, the SBT-040-WT-ATAC7 conjugate, the SBT-040-WT-ATAC8 conjugate, the SBT-040-WT-ATAC9 conjugate, the SBT-040-WT-ATAC10 conjugate, the SBT-040-WT-ATAC11 conjugate, the SBT-040-WT-ATAC12 conjugate, the SBT-040-VLPLL-ATAC1 conjugate, the SBT-040-VLPLL-ATAC2 conjugate, the SBT-040-VLPLL-ATAC3 conjugate, the SBT-040-VLPLL-ATAC4 conjugate, the SBT-040-VLPLL-ATAC5 conjugate, the SBT-040-VLPLL-ATAC6 conjugate, the SBT-040-VLPLL-ATAC7 conjugate, the SBT-040-VLPLL-ATAC8 conjugate, the SBT-040-VLPLL-ATAC9 conjugate, the SBT-040-VLPLL-ATAC10 conjugate, the SBT-040-VLPLL-ATAC11 conjugate, the SBT-040-VLPLL-ATAC12 conjugate, the SBT-040-DE-ATAC1 conjugate, the SBT-040-DE-ATAC2 conjugate, the SBT-040-DE-ATAC3 conjugate, the SBT-040-DE-ATAC4 conjugate, the SBT-040-DE-ATAC5 conjugate, the SBT-040-DE-ATAC6 conjugate, the SBT-040-DE-ATAC7 conjugate, the SBT-040-DE-ATAC8 conjugate, the SBT-040-DE-ATAC9 conjugate, the SBT-040-DE-ATAC10 conjugate, the SBT-040-DE-ATAC11 conjugate, the SBT-040-DE-ATAC12 conjugate, the SBT-040-AAA-ATAC1 conjugate, the SBT-040-AAA-ATAC2 conjugate, the SBT-040-AAA-ATAC3 conjugate, the SBT-040-AAA-ATAC4 conjugate, the SBT-040-AAA-ATAC5 conjugate, the SBT-040-AAA-ATAC6 conjugate, the SBT-040-AAA-ATAC7 conjugate, the SBT-040-AAA-ATAC8 conjugate, the SBT-040-AAA-ATAC9 conjugate, the SBT-040-AAA-ATAC10 conjugate, the SBT-040-AAA-ATAC11 conjugate, or the SBT-040-AAA-ATAC12 conjugate.

### EXAMPLE 8

### Treatment of Cancer by Administering a Conjugate

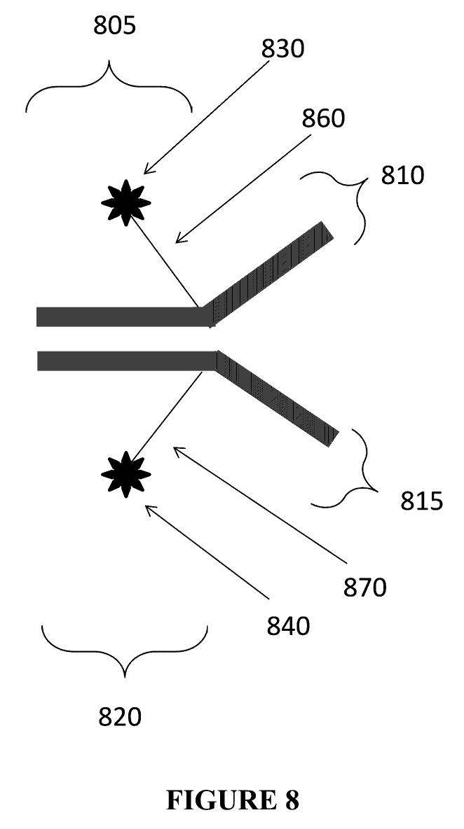

This example describes treatment of cancer with a conjugate. A human patient is diagnosed with a cancer. A conjugate as shown in the schematic of FIG. 8 is administered to the patient with a pharmaceutically acceptable carrier. FIG. 8 is a conjugate comprising an antibody construct and an immune stimulatory compound. The antibody construct is an antibody, which contains two heavy chains as shown in gray and two light chains as shown in light gray. The antibody comprises two antigen binding sites (810 and 815), and a portion of the heavy chains contain Fc domains (805 and 820). The immune-stimulatory compounds (830 and 840) are conjugated to the antibody by linkers (860 and 870).

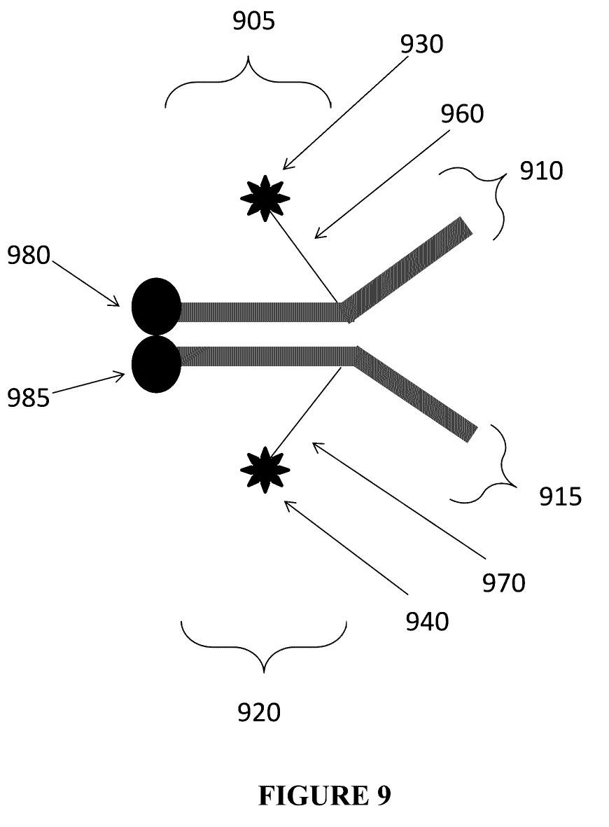

As another example, a human patient is diagnosed with a cancer. A conjugate as shown in the schematic of FIG. 9 is administered to the patient with a pharmaceutically acceptable carrier. FIG. 9 is a conjugate comprising an antibody construct, two targeting binding domains, and two immune stimulatory compounds. The antibody construct is an antibody, which contains two heavy chains as shown in gray and two light chains as shown in light gray. The antibody comprises two antigen binding sites (910 and 915), and a portion of the heavy chains contain Fc domains (905 and 920). The immune-stimulatory compounds (930 and 940) are conjugated to the antibody by linkers (960 and 970). The targeting binding domains are conjugated to the antibody (980 and 985).

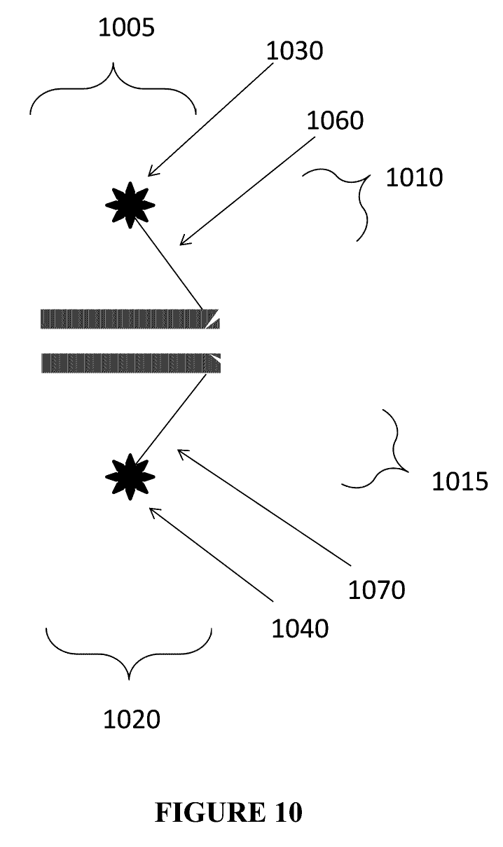

As an additional example, a human patient is diagnosed with a cancer. A conjugate as shown in the schematic of FIG. 10 is administered to the patient with a pharmaceutically acceptable carrier. FIG. 10 is a conjugate comprising an antibody construct and two immune stimulatory compounds. The antibody construct contains the Fc region of an antibody with the heavy chains shown in gray, and two scaffolds as shown in light gray. The antibody construct comprises two antigen binding sites (1010 and 1015) in the scaffolds, and a portion of the heavy chains contain Fc domains (1005 and 1020). The immune-stimulatory compounds (1030 and 1040) are conjugated to the antibody construct by linkers (1060 and 1070).

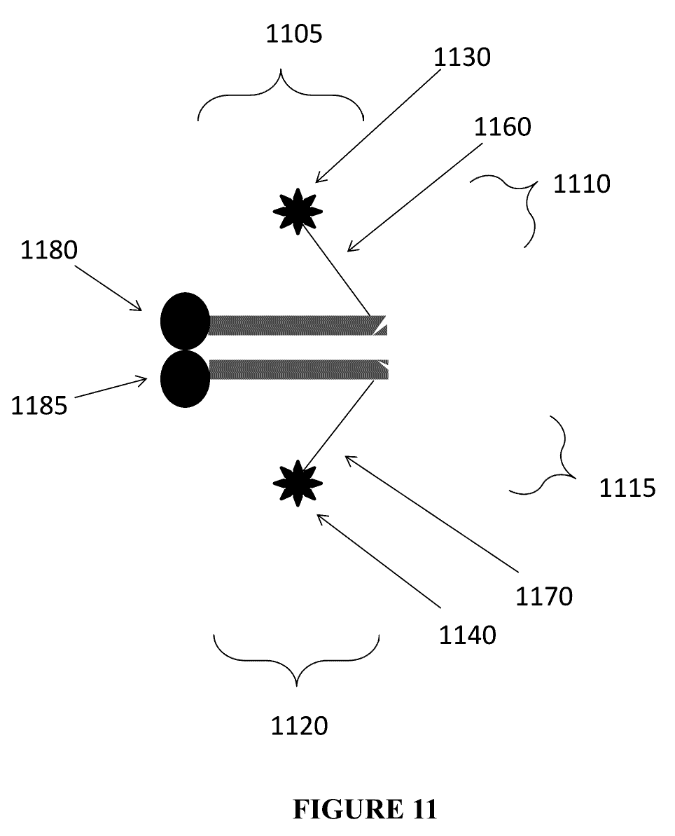

As another example, a human patient is diagnosed with a cancer. A conjugate as shown in the schematic of FIG. 11 is administered to the patient with a pharmaceutically acceptable carrier. FIG. 11 is a conjugate comprising an antibody construct, two targeting domains, and two immune stimulatory compounds. The antibody construct contains the Fc region of an antibody with the heavy chains shown in gray, and two scaffolds as shown in light gray. The antibody construct comprises two antigen binding sites (1110 and 1115) in the scaffolds, and a portion of the heavy chains contain Fc domains (1105 and 1120). The immune-stimulatory compounds (1130 and 1140) are conjugated to the antibody construct by linkers (1160 and 1170). The targeting binding domains are conjugated to the antibody construct (1180 and 1185).

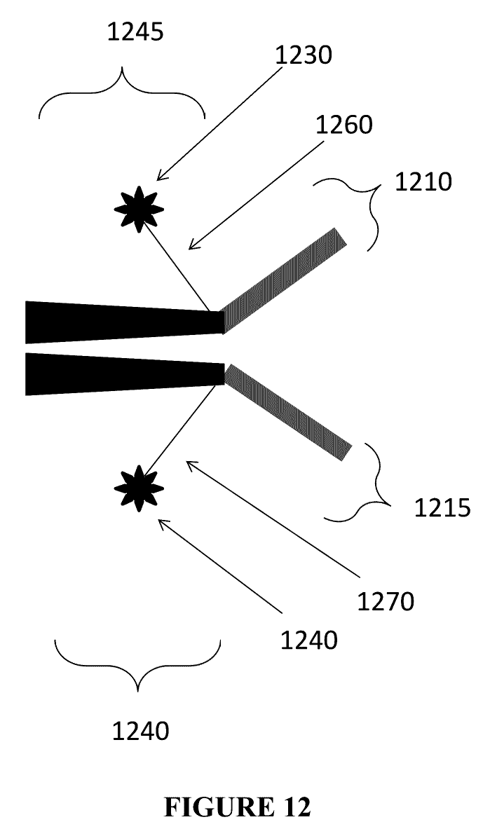

As another example, a human patient is diagnosed with a cancer. A conjugate as shown in the schematic of FIG. 12 is administered to the patient with a pharmaceutically acceptable carrier. FIG. 12 is a conjugate comprising an antibody construct and two immune stimulatory compounds. The antibody construct contains the F(ab′)2 region of an antibody with heavy chains shown in gray and light chains shown in light gray, and two scaffolds as shown in dark gray. The antibody construct comprises two antigen binding sites (1210 and 1215), and a portion of two scaffolds contain Fc domains (1240 and 1245). The immune-stimulatory compounds (1230 and 1240) are conjugated to the antibody construct by linkers (1260 and 1270).

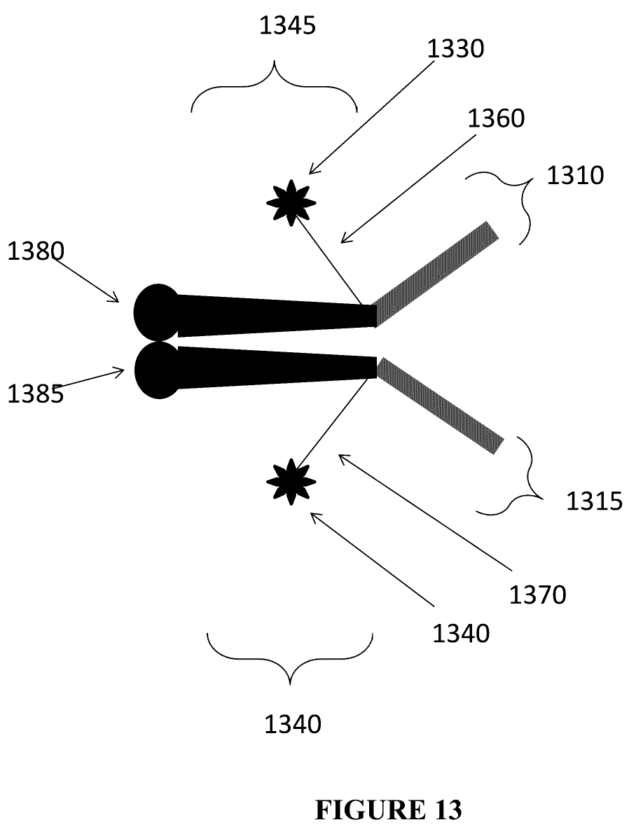

As another example, a human patient is diagnosed with a cancer. A conjugate as shown in the schematic of FIG. 13 is administered to the patient with a pharmaceutically acceptable carrier. FIG. 13 is a conjugate comprising an antibody construct, two targeting binding domains, and two immune stimulatory compounds. The antibody construct contains the F(ab′)2 region of an antibody with heavy chains shown in gray and light chains shown in light gray, and two scaffolds as shown in dark gray. The antibody construct comprises two antigen binding sites (1310 and 1315), and a portion of two scaffolds contain Fc domains (1340 and 1345). The immune-stimulatory compounds (630 and 640) are conjugated to the antibody construct by linkers (1360 and 1370). The targeting binding domains are conjugated to the antibody construct (1380 and 1385).

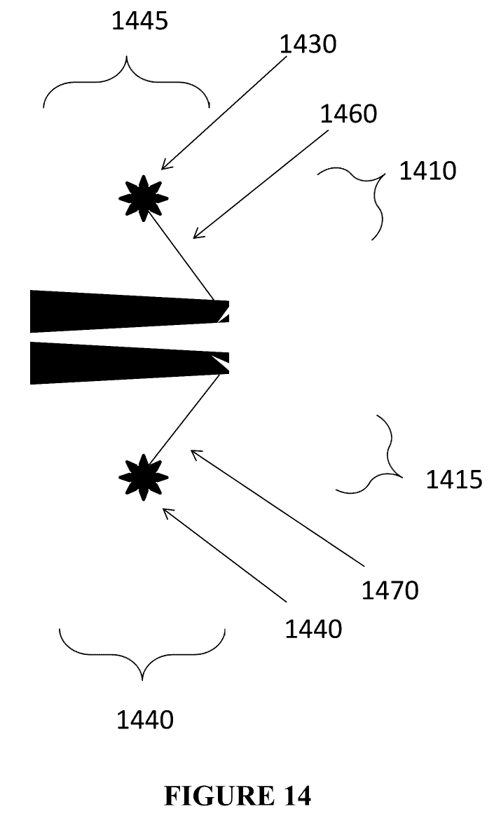

As another example, a human patient is diagnosed with a cancer. A conjugate as shown in the schematic of FIG. 14 is administered to the patient with a pharmaceutically acceptable carrier. FIG. 14 is a conjugate comprising an antibody construct, and two immune stimulatory compounds. The antibody construct contains two scaffolds as shown in light gray and two scaffolds as shown in dark gray. The antibody construct comprises two antigen binding sites (1410 and 1415), and a portion of the two dark gray scaffolds contain Fc domains (1440 and 1445). The immune-stimulatory compounds (1430 and 1440) are conjugated to the antibody construct by linkers (1460 and 1470).

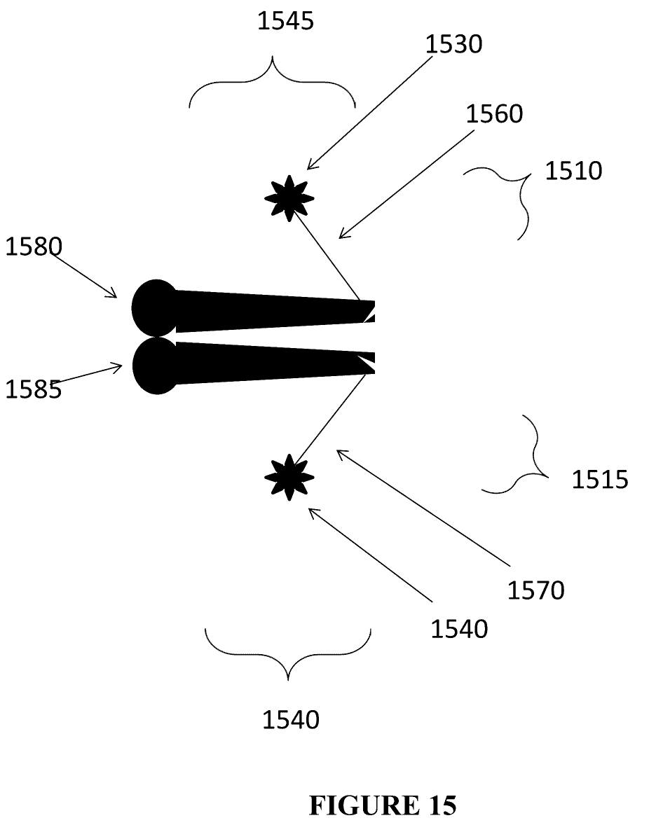

As another example, a human patient is diagnosed with a cancer. A conjugate as shown in the schematic of FIG. 15 is administered to the patient with a pharmaceutically acceptable carrier. FIG. 15 is a conjugate comprising an antibody construct, two targeting binding domains, and two immune stimulatory compounds. The antibody construct contains two scaffolds as shown in light gray and two scaffolds as shown in dark gray. The antibody construct comprises two antigen binding sites (1510 and 1515), and a portion of the two dark gray scaffolds contain Fc domains (1540 and 1545). The immune-stimulatory compounds (1530 and 1540) are conjugated to the antibody construct by linkers (1560 and 1570). The targeting binding domains are conjugated to the antibody construct (1580 and 1585).

### EXAMPLE 9

### Determination of K_{d} Values

K_{d} is measured by a radiolabeled antigen binding assay (RIA) performed with the Fab version of an antibody of interest and its antigen as described by the following assay.

Solution binding affinity of Fabs for antigen is measured by equilibrating the Fab with a minimal concentration of (¹²⁵I)-labeled antigen in the presence of a titration series of unlabeled antigen, then capturing bound antigen with an anti-Fab antibody-coated plate (See, e.g., Chen et al., J. Mol. Biol. 293:865-881 (1999)). To establish conditions for the assay, multi-well plates are coated overnight with 5 μg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant is added, and the plates are counted on a TOPCOUNT™ gamma counter (Packard) for ten minutes. Concentrations of each Fab that give less than or equal to 20% of maximal binding are chosen for use in competitive binding assays.

### EXAMPLE 10

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M−1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### EXAMPLE 11

### Lysine-Based Bioconjugation

The antibody construct is exchanged into an appropriate buffer, for example, phosphate, borate, PBS, Tris-Acetate at a concentration of about 2 mg/mL to about 10 mg/mL. An appropriate number of equivalents of the immune stimulatory compound-linker construct are added as a solution with stirring. Dependent on the physical properties of the immune stimulatory compound-linker construct, a co-solvent can be introduced prior to the addition of the immune stimulatory compound-linker construct to facilitate solubility. The reaction is stirred at room temperature for 2 hours to about 12 hours depending on the observed reactivity. The progression of the reaction is monitored by LC-MS. Once the reaction has been deemed complete, the remaining immune stimulatory compound-linker constructs are removed by applicable methods and the antibody construct-immune stimulatory compound conjugate is exchanged into the desired formulation buffer.

### EXAMPLE 12

### Cysteine-Based Bioconjugation

The antibody construct is exchanged into an appropriate buffer, for example, phosphate, borate, PBS, Tris-Acetate at a concentration of about 2 mg/mL to about 10 mg/mL with an appropriate number of equivalents of a reducing agent, for example, dithiothreitol or tris(2-carboxyethyl)phosphine. The resultant solution is stirred for an appropriate amount of time and temperature to effect the desired reduction. The immune stimulatory compound-linker construct is added as a solution with stirring. Dependent on the physical properties of the immune stimulatory compound-linker construct, a co-solvent can be introduced prior to the addition of the immune stimulatory compound-linker construct to facilitate solubility. The reaction is stirred at room temperature for about 1 hour to about 12 hours depending on the observed reactivity. The progression of the reaction is monitored by liquid chromatography-mass spectrometry (LC-MS). Once the reaction has been deemed complete, the remaining free immune stimulatory compound-linker construct is removed by applicable methods and the antibody construct-immune stimulatory compound conjugate is exchanged into the desired formulation buffer.

### EXAMPLE 13

### Determination of Molar Ratio

This example illustrates one method by which the molar ratio is determined. One microgram of antibody construct immune-stimulatory compound conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact antibody construct immune-stimulatory compound conjugates is calculated by the software, which can use top peak height at 25% for the calculation. This data is then imported into another program to calculate the molar ratio of the antibody construct immune-stimulatory compound conjugate such as Agilent molar ratio calculator.

### EXAMPLE 14

### Determination of Molar Ratio for SBT-040-G1WT Conjugated to a Cys-Targeted Compound

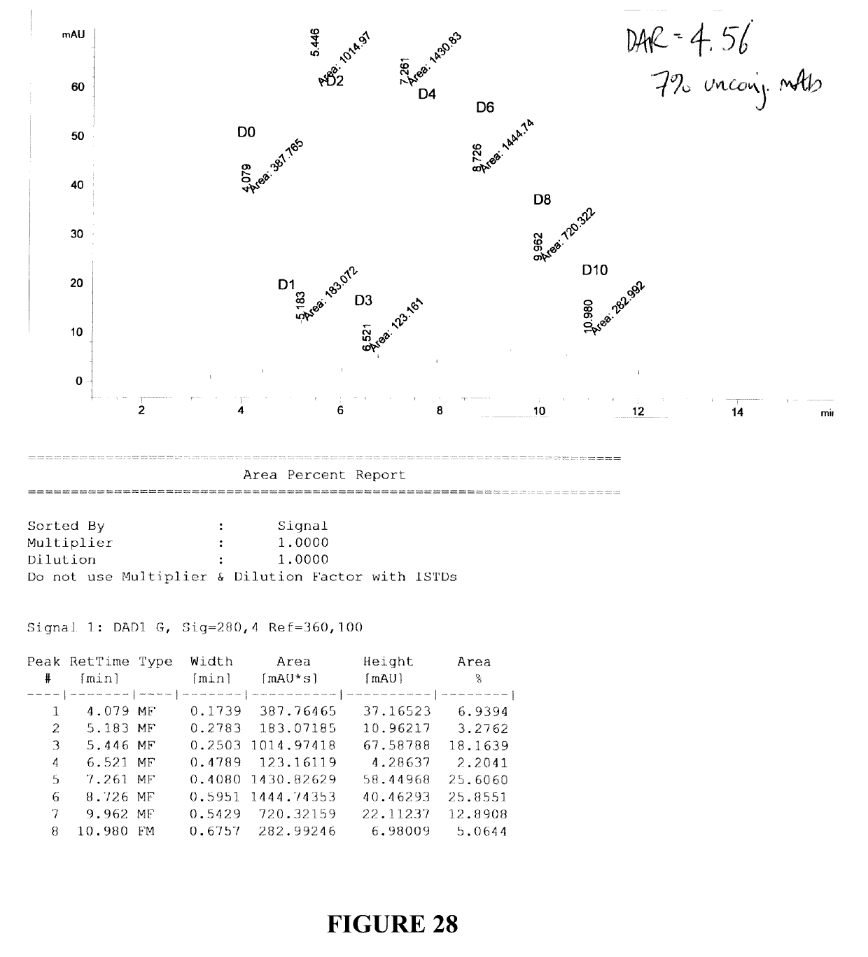

FIG. 28 shows HPLC analysis of SBT-040-G1WT conjugated to a Cys-targeted drug linker tool compound. First, 10 μL of a 5 mg/mL solution of the antibody-drug conjugate was injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 4.56 with 7% unconjugated antibody.

### EXAMPLE 15

### Determination of Molar Ratio for SBT-040-G1WT Conjugated to ATAC2

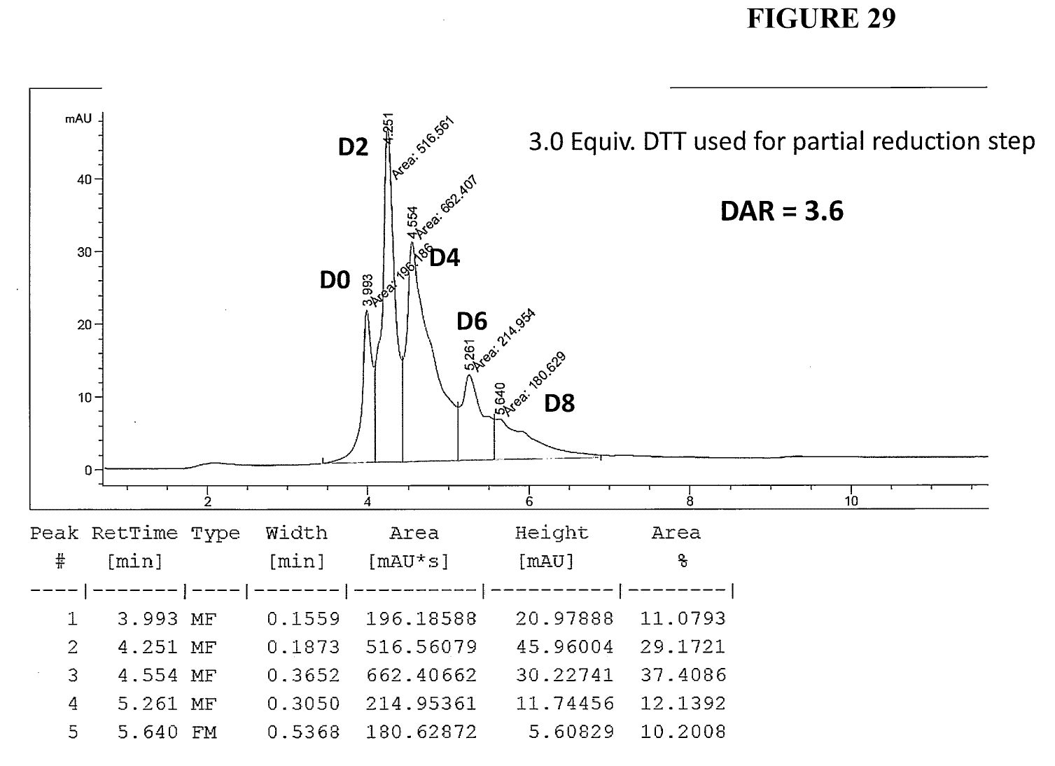

FIG. 29 shows HPLC analysis of SBT-040-G1WT conjugated to ATAC2, which is a cleavable Maleimide-Val-Ala-PABA-Gardiquimod linker. First, 10 μL of a 5 mg/mL solution of the antibody immune-stimulatory compound conjugate was injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 3.6.

### EXAMPLE 16

### Determination of Molar Ratio for SBT-040-G2WT Conjugated to ATAC2

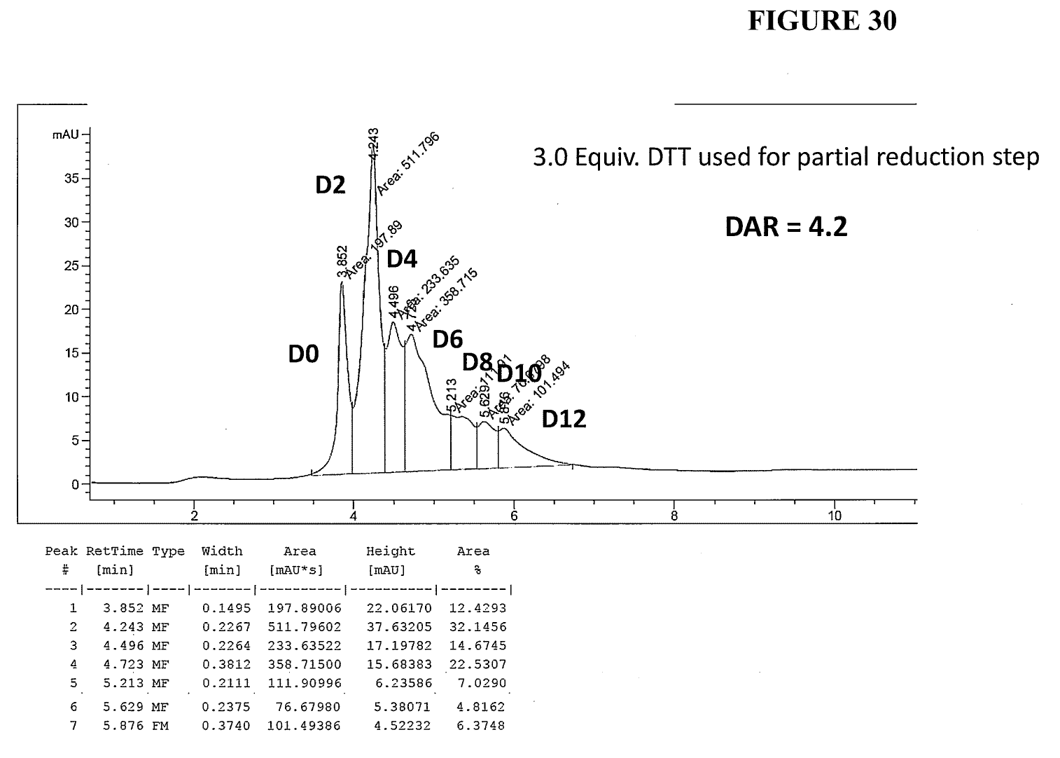

FIG. 30 shows HPLC analysis of SBT-040-G2WT conjugated to ATAC2, which is a cleavable Maleimide-Val-Ala-PABA-Gardiquimod linker. First, 10 μL of a 5 mg/mL solution of the antibody-immune stimulatory compound conjugate was injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 4.2.

### EXAMPLE 17

### Additional Method for Determination of Molar Ratio

Another method for determination of molar ratio is as follows. First, 10 μL of a 5 mg/mL solution of an antibody construct immune-stimulatory compound conjugate is injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A conjugate comprising:
a) an immune-stimulatory compound;
b) an antibody construct comprising an antigen binding domain and an Fc domain, wherein:
i) a Kd for binding of said antigen binding domain to a first antigen in a presence of said immune-stimulatory compound is less than about 100 nM and no greater than about 100 times a Kd for binding of said antigen binding domain to said first antigen in the absence of said immune-stimulatory compound, and
ii) a Kd for binding of said Fc domain to an Fc receptor in the presence of said immune-stimulatory compound is no greater than about 100 times a Kd for binding of said Fc domain to said Fc receptor in the absence of the immune stimulatory compound; and
c) a linker attaching said antibody construct to said immune-stimulatory compound, wherein said linker is covalently bound to said antibody construct and said linker is covalently bound to said immune-stimulatory compound, and wherein a molar ratio of immune-stimulatory compound to antibody construct is less than 8.

2. The conjugate of claim 1, wherein said antibody construct further comprises a targeting binding domain.

3. The conjugate of claim 2, wherein said targeting binding domain specifically binds a second antigen.

4. The conjugate of claim 3, wherein said targeting binding domain is conjugated to said antibody construct at a C-terminal end of said Fc domain.

5. The conjugate of claim 1, wherein said antigen binding domain is from an antibody or non-antibody scaffold.

6. The conjugate of claim 1, wherein said antigen binding domain is at least 80% homologous to an antigen binding domain from an antibody or non-antibody scaffold.

7. The conjugate of claim 1, wherein a complementarity determining region of the antigen binding domain comprises a light chain sequence that is at least 80% homologous to the SEQ ID NO: 27, a light chain sequence that is at least 80% homologous to SEQ ID NO: 28, a light chain sequence that is at least 80% homologous to SEQ ID NO: 29, a heavy chain sequence that is at least 80% homologous to SEQ ID NO: 23, a heavy chain sequence that is at least 80% homologous to SEQ ID NO: 24, or a heavy chain sequence that is at least 80% homologous to SEQ ID NO: 25.

8. The conjugate of claim 1, wherein a complementarity determining region of the antigen binding domain comprises a light chain sequence that is at least 80% homologous to the SEQ ID NO: 35, a light chain sequence that is at least 80% homologous to SEQ ID NO: 36, a light chain sequence that is at least 80% homologous to SEQ ID NO: 37, a heavy chain sequence that is at least 80% homologous to SEQ ID NO: 31, a heavy chain sequence that is at least 80% homologous to SEQ ID NO: 32, or a heavy chain sequence that is at least 80% homologous to SEQ ID NO: 33.

9. The conjugate of claim 1, wherein said first antigen is a tumor antigen.

10. The conjugate of claim 1, wherein said first antigen is CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Ley, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART1, Ras mutant, gp100, p53 mutant, PR1, bcr-abl, tyronsinase, survivin, PSA, hTERT, Sarcoma translocation breakpoints, EphA2, PAP, ML-IAP, AFP, ERG, NA17, PAX3, ALK, androgen receptor, cyclin B 1, polysialic acid, MYCN, RhoC, TRP-2, fucosyl GM1, mesothelin, PSCA, MAGE Al, sLe(animal), CYP1B1, PLAV1, GM3, BORIS, Tn, GloboH, ETV6-AML, NY-BR-1, RGS5, SART3, STn, Carbonic anhydrase IX, PAX5, OY-TESL Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1.

11. The conjugate of claim 1, wherein said first antigen is expressed on an immune cell.

12. The conjugate of claim 1, wherein said first antigen is CD40.

13. The conjugate of claim 1, wherein said antigen binding domain is a CD40 agonist.

14. The conjugate of claim 1, wherein said antibody construct is a human antibody or a humanized antibody.

15. The conjugate of claim 1, wherein said antibody construct comprises a light chain sequence that is at least 90% homologous to SEQ ID NO: 4, a variable domain sequence that is at least 90% homologous to SEQ ID NO: 6, a heavy chain sequence that is at least 90% homologous to SEQ ID NO: 15, a variable domain that is at least 90% homologous to SEQ ID NO: 20, a heavy chain sequence that is at least 90% homologous to SEQ ID NO: 16, a heavy chain sequence that is at least 90% homologous to SEQ ID NO: 17, or a heavy chain sequence that is at least 90% homologous to SEQ ID NO: 18.

16. The conjugate of claim 1, wherein said Fc domain is an Fc domain variant comprising at least one amino acid residue change as compared to a wild type sequence of said Fc domain.

17. The conjugate of claim 1, wherein the linker is a peptide.

18. The conjugate of claim 1, wherein said Kd for binding of said antigen binding domain to said first antigen in the presence of said immune-stimulatory compound is less than about 100 nM and is no greater than about 10 times the Kd of the binding of the antigen binding domain to said first antigen in the absence of the immune-stimulatory compound; and said Kd for binding of said Fc domain to said Fc receptor in the presence of said immune-stimulatory compound is no greater than about 10 times said Kd for the binding of said Fc domain to said Fc receptor in the absence of said immune stimulatory compound.

19. The conjugate of claim 1, wherein said molar ratio of immune-stimulatory compound to antibody is less than 5.

20. The conjugate of claim 1, wherein said conjugate is in a pharmaceutical formulation.
